(19) United States
(12) Patent Application Publication
(10) Pub. No.: US 2010/0267728 A1
(43) Pub. Date: Oct. 21, 2010

(54) 3-SUBSTITUTED-1,2,3-TRIAZIN-4-ONE'S AND 3 SUBSTITUTED 1,3-PYRIMIDINONE'S FOR ENHANCING GLUTAMATERGIC SYNAPTIC RESPONSES

(75) Inventors: Rudolf Mueller, Foothill Ranch, CA (US); Leslie J. Street, Laguna Niguel, CA (US); Stanislaw Rachwal, Irvine, CA (US); Kashinatham Alisala, San Diego, CA (US)

Correspondence Address: COLEMAN SUDOL SAPONE, P.C., 714 COLORADO AVENUE, BRIDGE PORT, CT, 06605-1601, US

(73) Assignee: CORTEX PHARMACEUTICALS, INC., Irvine, CA (US)

(21) Appl. No.: 12/733,822

(22) PCT Filed: Sep. 19, 2008
(86) PCT No.: PCT/US2008/010877

§ 371 Date: May 27, 2010

Related U.S. Application Data
(60) Provisional application No. 60/994,548, filed on Sep. 20, 2007.

(51) Int. Cl. A61K 31/53; C07D 487/14; C07D 239/90; A61K 31/517; A61P 25/16; A61P 25/24; A61P 25/28; A61P 25/20; A61P 25/18; A61P 11/00; A61P 25/00; A61P 25/04

(52) U.S. Cl. 514/243; 544/184; 544/251; 514/267

(57) ABSTRACT

This invention relates to compounds, pharmaceutical compositions and methods for use in the prevention and treatment of cerebral insufficiency, including enhancement of receptor functioning in synapses in brain networks responsible for basic and higher order behaviors. These brain networks, which are involved in regulation of breathing, and cognitive abilities related to memory impairment, such as is observed in a variety of dementias, in imbalances in neuronal activity between different brain regions, as is suggested in disorders such as Parkinson's disease, schizophrenia, respiratory depression, sleep apneas, attention deficit hyperactivity disorder and affective or mood disorders, and in disorders wherein a deficiency in neurotrophic factors is implicated, as well as in disorders of respiration such as overdose of an alcohol, an opiate, an opioid, a barbiturate, an anesthetic, or a nerve toxin, or where the respiratory depression results form a medical condition such as central sleep apnea, stroke-induced central sleep apnea, obstructive sleep apnea, congenital hypoventilation syndrome, obesity hypoventilation syndrome, sudden infant death syndrome, Rett syndrome, spinal cord injury, traumatic brain injury, Cheney-Stokes respiration, Ondines curse, Prader-Willi's syndrome and drowning. In a particular aspect, the invention relates to compounds useful for treatment of such conditions, and methods of using these compounds for such treatment.

### RELATED APPLICATIONS

The present application claims priority from U.S. Provisional Application Ser. No. 60/994,558, filed Sep. 20, 2007, of identical title, the entire contents of which application are incorporated by reference herein.

### FIELD OF THE INVENTION

This invention relates to compounds, pharmaceutical compositions and methods for use in the prevention and treatment of cerebral insufficiency, including enhancement of receptor functioning in synapses in brain networks responsible for various behaviors. These brain networks are involved in basic functions such as breathing, to more complex functions such as memory and cognition. Imbalances in neuronal activities between different brain regions may lead to a number of disorders, including psychiatric and neurological disorders, including memory impairment, Parkinson's disease, schizophrenia, attention deficit and affective or mood disorders, respiratory depression and in disorders wherein a deficiency in neurotrophic factors is implicated. In a particular aspect, the invention relates to compounds useful for treatment of such conditions, and methods of using these compounds for such treatment.

### BACKGROUND OF THE INVENTION

The release of glutamate at synapses at many sites in mammalian forebrain stimulates two classes of postsynaptic, ionotropic receptors. These classes are usually referred to as AMPA/quisqualate and N-methyl-D-aspartic acid (NMDA) receptors. AMPA/quisqualate receptors mediate a voltage independent fast excitatory post-synaptic current (the fast EPSC), whereas NMDA receptors generate a voltage-dependent, slow excitatory current. Studies carried out in slices of hippocampus or cortex, indicate that the AMPA receptor mediated fast EPSC is generally the dominant component by far at most glutamatergic synapses.

AMPA receptors are expressed throughout the central nervous system. These receptors are found in high concentrations in the superficial layers of neocortex, in each of the major synaptic zones of hippocampus, and in the striatal complex, as reported by Monaghan et al., in Brain Research 324:160-164 (1984). Studies in animals and humans indicate that these structures organize complex perceptual-motor processes and provide the substrates for higher-order behaviors. Thus, AMPA receptors mediate transmission in those brain networks responsible for a host of cognitive activities. In addition, AMPA receptors are expressed in brain regions that regulate the inspiratory drive responsible for control of breathing (Paarmann et al, Journal of Neurochemistry, 74: 1335-1345 (2000).

For the reasons set forth above, drugs that modulate and thereby enhance the functioning of AMPA receptors could have significant benefits for cognitive and intellectual performance. Such drugs should also facilitate memory encoding. Experimental studies, such as those reported by Arai and Lynch, Brain Research 598:173-184 (1992), indicate that increasing the size of AMPA receptor-mediated synaptic response(s) enhances the induction of long-term potentiation (LTP). LTP is a stable increase in the strength of synaptic contacts that follows repetitive physiological activity of a type known to occur in the brain during learning.

Compounds that enhance the functioning of the AMPA subtype of glutamate receptors facilitate the induction of LTP and the acquisition of learned tasks as measured by a number of paradigms. See, for example, Granger et al., Synapse 15:326-329 (1993); Staubli et al., PNAS 91:777-781 (1994); Arai et al., Brain Res. 638:343-346 (1994); Staubli et al., PNAS 91:11158-11162 (1994); Shors et al., Neurosci. Let. 186:153-156 (1995); Larson et al., J. Neurosci. 15:8023-8030 (1995); Granger et al., Synapse 22:332-337 (1996); Arai et al., JPET 278:627-638 (1996); Lynch et al., Internat. Clin. Psychopharm. 11:13-19 (1996); Lynch et al., Exp. Neurology 145:89-92 (1997); Ingvar et al., Exp. Neurology 146:553-559 (1997); Hampson, et al., J. Neurosci. 18:2748-2763 (1998); Porrino et al., PLoS Biol 3(9): 1-14 (2006) and Lynch and Rogers, U.S. Pat. No. 5,747,492. There is a considerable body of evidence showing that LTP is the substrate of memory. For example, compounds that block LTP interfere with memory formation in animals, and certain drugs that disrupt learning in humans antagonize the stabilization of LTP, as reported by del Cerro and Lynch, Neuroscience 49: 1-6 (1992). Learning a simple task induces LTP in hippocampus that occludes LTP generated by high frequency stimulation (Whitlock et al., Science 313:1093-1097 (2006)) and a mechanism that maintains LTP sustains spatial memory (Pastalkova, et al., Science 313:1141-1144 (2006)). Of significant importance to the field of learning is the finding that in vivo treatments with a positive AMPA-type glutamate receptor modulator restores stabilization of basal dendritic LTP in middle-aged animals (Rex, et al., J. Neurophysiol. 96:677-685 (2006)).

Drugs that enhance the functioning of the AMPA receptor can effectively reverse opioid- and barbiturate-induced respiratory depression without reversing the analgesic response (Ren et al, American Journal of Respiratory and Critical Care Medicine, 174: 1384-1391 (2006). Therefore these drugs may be useful in preventing or reversing opioid-induced respiratory depression and for alleviating other forms of respiratory depression including sedative use and sleep apnea. Excitatory synaptic transmission provides a major pathway by which neurotrophic factors are increased within specific brain regions. As such, potentiation of AMPA receptor function by modulators has been found to increase levels of neurotrophins, particularly brain derived neurotrophic factor, or BDNF. See, for example, Lauterborn, et al., J. Neurosci. 20:8-21 (2000); Gall, et al., U.S. Pat. No. 6,030,968; Lauterbom, et al., JPET 307:297-305 (2003); and Mackowiak, et al., Neuropharmacolozv 43:1-10 (2002). Other studies have linked BDNF levels to a number of neurological disorders, such as Parkinson's disease, Attention Deficit Hyperactivity Disorder (ADHD), autism, Fragile-X Syndrome, and Rett Syndrome (RTT). See, for example, O'Neill, et al., Eur. J. Pharmacol. 486:163-174 (2004); Kent, et al., Mol. Psychiatry. 10:939-943 (2005); Riikonen, et al., J. Child Neurol. 18:693-697 (2003) and Chang, et al., Neuron 49:341-348 (2006). Thus, AMPA receptor potentiators may be useful for the treatment of these, as well as other, neurological diseases that are the result of a glutamatergic imbalance or a deficit in neurotrophic factors.

A prototype for a compound that selectively facilitates the AMPA receptor has been described by Ito et al., J. Physiol. 424:533-543 (1990). These authors found that the nootropic drug aniracetam (N-anisoyl-2-pyrrolidinone) increases currents mediated by brain AMPA receptors expressed in Xenopus oocytes without affecting responses by γ-aminobutyric acid (GABA), kainic acid (KA), or NMDA receptors. Infusion of aniracetam into slices of hippocampus was also shown to substantially increase the size of fast synaptic potentials without altering resting membrane properties. It has since been confirmed that aniracetam enhances synaptic responses at several sites in hippocampus, and that it has no effect on NMDA-receptor mediated potentials (Staubli et al., Psychobiology 18:377-381 (1990) and Xiao et al., Hip pocampus 1:373-380 (1991)).

Aniracetam has been found to have an extremely rapid onset and washout, and can be applied repeatedly with no apparent lasting effects, which are desirable features for behaviorally-relevant drugs. Aniracetam does present several disadvantages, however. The peripheral administration of aniracetam is not likely to influence brain receptors. The drug works only at high concentrations (approx. 1000 μM), and about 80% of the drug is converted to anisoyl-GABA following peripheral administration in humans (Guenzi and Zanetti, J. Chromatogr. 530:397-406 (1990)). The metabolite, anisoyl-GABA, has been found to have less activity than aniracetam. In addition to these issues, aniracetam has putative effects on a plethora of other neurotransmitter and enzymatic targets in the brain, which makes uncertain the mechanism of any claimed therapeutic drug effect. See, for example, Himori, et al., Pharmacology Biochemistry and Behavior 47:219-225 (1994); Pizzi et al., J. Neurochem. 61:683-689 (1993); Nakamura and Shirane, Eur. J. Pharmacol. 380: 81-89 (1999); Spignoli and Pepeu, Pharmacol. Biochem. Behav. 27:491-495 (1987); Hall and Von Voigtlander, Neuropharmacology 26:1573-1579 (1987); and Yoshimoto et al., J. Pharmacobiodyn. 10:730-735 (1987).

A class of AMPA receptor-enhancing compounds that does not display the low potency and inherent instability characteristic of aniracetam has been described (Lynch and Rogers, U.S. Pat. No. 5,747,492). These compounds, termed “Ampakines”^{R}, can be substituted benzamides which include, for example, 6-(piperidin-1-ylcarbonyl)quinoxaline (CX516; Ampalex^{R}). Typically, they are chemically more stable than aniracetam and show improved bio-availability. CX516 is active in animal tests used to detect efficacious drugs for the treatment of memory disorders, schizophrenia, and depression. In three separate clinical trials, CX516 showed evidence for efficacy in improving various forms of human memory (Lynch et al., Internat. Clin. Psychopharm. 11:13-19 (1996); Lynch et al., Exp. Neurology 145:89-92 (1997); Ingvar et al., Exp. Neurology 146:553-559 (1997)).

Another class of Ampakines, benzoxazines, has been discovered to have very high activity in in vitro and in vivo models for assessing the probability of producing cognition enhancement (Rogers and Lynch; U.S. Pat. No. 5,736,543). The substituted benzoxazines are rigid benzamide analogues with different receptor modulating properties from the flexible benzamide, CX516.

Further previously disclosed structures contained a 1,3-benzoxazine-4-one pharmacophore and were substituted on the benzene portion by heteroatoms, such as nitrogen or oxygen (U.S. Pat. Nos. 5,736,543 and 5,962,447), by substituted alkyl groups (U.S. Pat. Nos. 5,650,409 and 5,783,587), or un-substituted (WO 99/42456). In WO 08/085,505 a series of 7,8-dihydro-3H-[1,3]oxazino[6,5-g][1,2,3]benzotriazine-4,9-dione compounds are disclosed as having Ampakine activity. Now a new class of substituted benzotriazinone and substituted benzopyrimidinone compounds (I, X and Y═C or N) have been discovered that display significant activity on hippocampal synaptic responses and neuronal whole cell currents mediated by AMPA receptors. 3-Substituted benzo[d]1,2,3-triazin-4-ones and 3-substituted benzo[d]1,3-pyrimidin-4-ones are potent AMPA receptor modulators with high potency at the AMPA receptor and are significantly more metabolically stable than the corresponding benzoxazinone and 7,8-dihydro-3H-[1,3]oxazino[6,5-g][1,2,3]benzotriazine-4,9-dione compounds leading to improved oral activity. These compounds are disclosed herein.

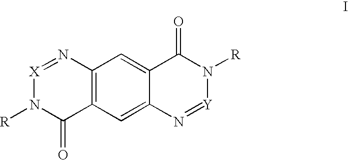

### SUMMARY OF THE INVENTION

The present invention includes, in one aspect, a compound as shown by structure I, and described in Section II of the Detailed Description, which follows. Administration of compounds of this class has been found to increase synaptic responses mediated by AMPA receptors. The compounds of the present invention are significantly more potent than previously described compounds in increasing AMPA receptor function in primary neuronal cultures and in slices of rat hippocampus, and in enhancing cognitive performance, such as performance in a delayed match to sample task. This unexpected activity translates into pharmaceutical compounds and corresponding methods of use, including treatment methods, which utilize significantly lower concentrations (on a mole-to-mole basis) of the present compounds compared to prior art compositions.

The ability of the compounds of the invention to increase AMPA receptor-mediated responses makes the compounds useful for a variety of purposes. These include facilitating the learning of behaviors dependent upon glutamate receptors, treating conditions in which AMPA receptors or synapses utilizing these receptors are reduced in numbers or efficiency, and enhancing excitatory synaptic activity in order to restore an imbalance between brain subregions or increase the levels of neurotrophic factors.

In another aspect, the invention includes a method for the treatment of a mammalian subject suffering from a hypoglutamatergic condition, or from a deficiency in the number or strength of excitatory synapses, or in the number of AMPA receptors, such that memory or other cognitive functions are impaired. Such conditions may also cause a cortical/striatal imbalance, leading to schizophrenia or schizophreniform behavior.

In another aspect, the invention includes a method for reducing or inhibiting respiratory depression in a subject having respiratory depression, comprising administering to the subject an amount of a compound of the invention, the amount being sufficient to reduce or inhibit respiratory depression. In one embodiment of the invention, the subject is a human. In another embodiment, the subject is a mammal. Also claimed is a method for reducing or inhibiting respiratory depression comprising administering to the subject an amount of a compound of the invention in combination with an opioid analgesic; examples of such opiates include but are not limited to, alfentanil and fentanyl.

In another aspect, the invention includes a method for reducing or inhibiting breathing-related sleep disorders or sleep apnea in a subject having sleep apnea, comprising administering to the subject an amount of a compound of the invention, the amount being sufficient to reduce or inhibit the breathing related sleep disorder.

According to the methods, such a subject is treated with an effective amount of a compound as shown by structure I, and described in Section II of the Detailed Description, following, in a pharmaceutically acceptable carrier. These and other objects and features of the invention will become more fully apparent when the following detailed description of the invention is read in conjunction with the accompanying drawings.

### DETAILED DESCRIPTION OF THE INVENTION

### Definitions

The terms below have the following definitions unless indicated otherwise. Other terms that are used to describe the present invention have the same definitions as those terms are generally used by those skilled in the art.

The term “alkyl” is used herein to refer to a fully saturated monovalent radical containing carbon and hydrogen (up to 10 carbon atoms), and which may be a straight chain, branched or cyclic. Examples of alkyl groups are methyl, ethyl, n-butyl, n-heptyl, isopropyl, 2-methylpropyl.

The term “cycloalkyl” is used herein to refer to a fully saturated monovalent radical containing up to 8 carbons and hydrogen in a ring. Examples of cycloalkyl groups are cyclopropyl, cyclobutyl, cyclopentyl, cyclohexyl and cycloheptyl.

The term “bicycloalkyl” is used herein to refer to a fully saturated monovalent radical containing up to 10 carbons and hydrogen in a bicyclic ring. Examples of bicycloalkyl groups are bicyclo[2.2.2]octyl, bicyclo[2.2.1]heptyl and bicyclo[2.2.3]nonyl and bicylo[3.2.1]octyl.

The term “azabicycloalkyl” is used herein to refer to a fully saturated monovalent radical containing up to 10 carbons and hydrogen and 1 nitrogen atom in a bicyclic ring. Examples of azabicycloalkyl groups a include 1-azabicyclo[2.2.2]octyl, 2-azabicyclo[2.2.2]octyl, 1-azabicyclo[2.2.1]heptyl, 2-azabicyclo[2.2.1]heptyl and 1-azabicylo[3.2.1]octyl.

The term “alkenyl” is used herein to refer to a monovalent radical containing carbon and hydrogen (up to 10 carbon atoms) that contains one or two sites of un-saturation, and which may be a straight chain, branched or cyclic. Examples of alkenyl groups are ethenyl, n-butenyl, n-heptenyl, isopropenyl, cyclopentenyl, cyclopentenylethyl and cyclohexenyl.

The term “alkynyl” is used therein to refer to a monovalent radical containing carbon and hydrogen (up to 10 carbon atoms) that contains at least one triple bond between carbon atoms within the group and which may be a straight chain, branched or cyclic.

The terms “substituted alkyl”, “substituted alkenyl” and “substituted alkynyl” refers to alkyl, alkenyl and alkynyl groups as just described which include one or more functional groups as substituents such as lower alkyl containing 1-6 carbon atoms, aryl, substituted aryl, acyl, halogen (F, Cl, Br, I, e.g., alkyl halos, e.g., CF₃), amido, thioamido cyano, nitro, alkynyl, azido, hydroxy, alkoxy, alkoxyalkyl, amino, alkyl and dialkyl-amino, acylamino, acyloxy, aryloxy, aryloxyalkyl, carboxyalkyl, carboxamido, thio, thioethers, both saturated and unsaturated cyclic hydrocarbons, heterocycles and the like.

The term “aryl” refers to a substituted or unsubstituted monovalent aromatic radical having a single ring (e.g., phenyl) or multiple condensed rings (e.g., naphthyl). Other examples include heterocyclic aromatic (heteroaromatic) ring groups having one or more nitrogen, oxygen, or sulfur atoms in the ring, such as oxazolyl, isoxazolyl, pyrazolyl, thiazolyl, thiadiazolyl, triazolyl, tetrazolyl, pyridazinyl, pyrimidyl, benzofuryl, benzothienyl, benzimidazolyl, benzotriazolyl, benzoxazolyl, benzothiazolyl, quinolyl, isoquinolyl, imidazolyl, furyl, pyrrolyl, pyridyl, thienyl and indolyl.

The term “substituted” as used in the term “substituted aryl, substituted aromatic, substituted heteroaryl, or substituted heteroaromatic” herein signifies that one or more substituents may be present, said substituents being selected from atoms and groups, which when present do not prevent the compound from functioning as a potentiator of AMPA receptor function. Examples of substituents that may be present in a substituted aromatic or heteroaromatic group include, but are not limited to, groups such as (C₁-C₇) alkyl, (C₁-C₇) acyl, aryl, heteroaryl, substituted aryl and heteroaryl, halogen, cyano, nitro, amido (optionally substituted with one or two C₁-C₇ alkyl groups), thioamido (optionally substituted with one or two C₁-C₇ alkyl groups), azido, alkynyl, (C₁-C₇) alkylhalos (e.g., CF₃), hydroxy, (C₁-C₇) alkoxy, (C₂-C₈) alkoxyalkyl, amino, (C₁-C₇) alkyl and dialkyl amino, (C₁-C₇) acylamino, (C₁-C₇) acyloxy, aryloxy, (C₁-C₇) aryloxyalkyl, (C₁-C₇) carboxyalkyl, carboxamido, thio, (C₁-C₇) thioethers, both saturated and unsaturated (C₃-C₈) cyclic hydrocarbons, (C₃-C₈) heterocycles and the like. It is noted that each of the substituents disclosed herein may themselves be substituted.

“Heterocycle” or “heterocyclic” refers to a carbocylic ring wherein one or more carbon atoms have been replaced with one or more heteroatoms such as nitrogen, oxygen or sulfur. Examples of heterocycles include, but are not limited to, piperidine, pyrrolidine, morpholine, thiomorpholine, piperazine, tetrahydrofuran, tetrahydropyran, 2-pyrrolidinone, 8-valerolactam, 5-valerolactone and 2-ketopiperazine. “5-ring heterocycles” refers to heterocycles containing 5 atoms within the heterocyclic ring. “6-ring heterocycles” refers to heterocycles containing 6 atoms within the heterocyclic ring. “5-ring heteroaromatics” refers to heteroaromatics containing 5 atoms within the heteroaromatic ring. “6-ring heteroaromatics” refers to heteroaromatics containing 6 atoms within the heteroaromatic ring. Heterocycles and heteroaromatics may be unsubstituted or substituted as otherwise described herein.

The term “substituted heterocycle” refers to a heterocycle as just described that contains one or more functional groups such as lower alkyl, acyl, aryl, cyano, halogen, amido, thioamido, azido, hydroxy, alkoxy, alkoxyalkyl, amino, alkyl and dialkyl-amino, acylamino, acyloxy, aryloxy, aryloxyalkyl, carboxyalkyl, carboxamido, thio, thioethers, both saturated and unsaturated cyclic hydrocarbons, heterocycles and the like, as otherwise described herein.

The term “compound” is used herein to refer to any specific chemical compound disclosed herein. Within its use in context, the term generally refers to a single compound, but in certain instances may also refer to stereoisomers and/or optical isomers (including enantiopure compounds, enantiomerically enriched compounds and racemic mixtures) of disclosed compounds.

The term “effective amount” refers to the amount of a selected compound of formula I that is used within the context of its intended use to effect an intended result, for example, to enhance glutamatergic synaptic response by increasing AMPA receptor activity. The precise amount used will vary depending upon the particular compound selected and its intended use, the age and weight of the subject, route of administration, and so forth, but may be easily determined by routine experimentation. In the case of the treatment of a condition or disease state, an effective amount is that amount which is used to effectively treat the particular condition or disease state.

The term “pharmaceutically acceptable carrier” refers to a carrier or excipient which is not unacceptably toxic to the subject to which it is administered. Pharmaceutically acceptable excipients are described at length by E. W. Martin, in “Remington's Pharmaceutical Sciences.”

A “pharmaceutically acceptable salt” of an amine compound, such as those contemplated in the current invention, is an ammonium salt having as counter ion an inorganic anion such as chloride, bromide, iodide, sulfate, sulfite, nitrate, nitrite, phosphate, and the like, or an organic anion such as acetate, malonate, pyruvate, propionate, fumarate, cinnamate, tosylate, and the like.

The term “patient” or “subject” is used throughout the specification to describe an animal, generally a mammalian animal, including a human, to whom treatment or use with the compounds or compositions according to the present invention is provided. For treatment or use with/or of those conditions or disease states which are specific for a specific animal (especially, for example, a human subject or patient), the term patient or subject refers to that particular animal.

The term “sensory motor problems” is used to describe a problem which arises in a patient or subject from the inability to integrate external information derived from the five known senses in such a way as to direct appropriate physical responses involving movement and action.

The term “cognitive task” or “cognitive function” is used to describe an endeavor or process by a patient or subject that involves thought or knowing. The diverse functions of the association cortices of the parietal, temporal and frontal lobes, which account for approximately 75% of all human brain tissue, are responsible for much of the information processing that goes on between sensory input and motor output. The diverse functions of the association cortices are often referred to as cognition, which literally means the process by which we come to know the world. Selectively attending to a particular stimulus, recognizing and identifying these relevant stimulus features and planning and experiencing the response are some of the processes or abilities mediated by the human brain which are related to cognition.

The term “brain network” is used to describe different anatomical regions of the brain that communicate with one another via the synaptic activity of neuronal cells.

The term “AMPA receptor” refers to an aggregate of proteins found in some membranes, which allows positive ions to cross the membrane in response to the binding of glutamate or AMPA (DL-α-amino-3-hydroxy-5-methyl-4-isoxazolepropionic acid), but not NMDA.

The term “excitatory synapse” is used to describe a cell-cell junction at which release of a chemical messenger by one cell causes depolarization of the external membrane of the other cell. An excitatory synapse describes a postsynaptic neuron which has a reversal potential that is more positive than the threshold potential and consequently, in such a synapse, a neurotransmitter increases the probability that an excitatory post synaptic potential will result (a neuron will fire producing an action potential). Reversal potentials and threshold potentials determine postsynaptic excitation and inhibition. If the reversal potential for a post synaptic potential (“PSP”) is more positive than the action potential threshold, the effect of a transmitter is excitatory and produces an excitatory post synaptic potential (“EPSP”) and the firing of an action potential by the neuron. If the reversal potential for a post synaptic potential is more negative than the action potential threshold, the transmitter is inhibitory and may generate inhibitory post synaptic potentials (IPSP), thus reducing the likelihood that a synapse will fire an action potential. The general rule for postsynaptic action is: if the reversal potential is more positive than threshold, excitation results; inhibition occurs if the reversal potential is more negative than threshold. See, for example, Chapter 7, NEUROSCIENCE, edited by Dale Purves, Sinauer Associates, Inc., Sunderland, Mass. 1997.

The term “motor task” is used to describe an endeavor taken by a patient or subject that involves movement or action.

The term “perceptual task” is used to describe an act by a patient or subject of devoting attention to sensory inputs.

The term “synaptic response” is used to describe biophysical reactions in one cell as a consequence of the release of chemical messengers by another cell with which it is in close contact.

The term “hypoglutamatergic condition” is used to describe a state or condition in which transmission mediated by glutamate (or related excitatory amino acids) is reduced to below normal levels. Transmission consists of the release of glutamate, binding to post synaptic receptors, and the opening of channels integral to those receptors. The end point of the hypoglutamatergic condition is reduced excitatory post synaptic current. It can arise from any of the three above noted phases of transmission. Conditions or disease states which are considered hypoglutamatergic conditions and which can be treated using the compounds, compositions and methods according to the present invention include, for example, loss of memory, dementia, depression, attention disorders, sexual dysfunction, movement disorders, including Parkinson's disease, schizophrenia or schizophreniform behavior, memory and learning disorders, including those disorders which result from aging, trauma, stroke and neurodegenerative disorders, such as those associated with drug-induced states, neurotoxic agents, Alzheimer's disease and aging, respiratory depression and sleep apnea. These conditions are readily recognized and diagnosed by those of ordinary skill in the art.

The term “cortico-striatal imbalance” is used to describe a state in which the balance of neuronal activities in the interconnected cortex and underlying striatal complex deviates from that normally found. ‘Activity’ can be assessed by electrical recording or molecular biological techniques. Imbalance can be established by applying these measures to the two structures or by functional (behavioral or physiological) criteria.

The term “affective disorder” or “mood disorder” describes the condition when sadness or elation is overly intense and continues beyond the expected impact of a stressful life event, or arises endogenously. As used herein, the term “effective disorder” embraces all types of mood disorders as described in, for example, Diagnostic and Statistical Manual of Mental Disorders, Fourth Edition (DSM IV), pages 317-391.

The term “schizophrenia” is used to describe a condition which is a common type of psychosis, characterized by a disorder in the thinking processes, such as delusions and hallucinations, and extensive withdrawal of the individual's interest from other people and the outside world, and the investment of it in his or her own. Schizophrenia is now considered a group of mental disorders rather than a single entity, and distinction is made between reactive and process schizophrenias. As used herein, the term schizophrenia or “schizophreniform” embraces all types of schizophrenia, including ambulatory schizophrenia, catatonic schizophrenia, hebephrenic schizophrenia, latent schizophrenia, process schizophrenia, pseudoneurotic schizophrenia, reactive schizophrenia, simple schizophrenia, and related psychotic disorders which are similar to schizophrenia, but which are not necessarily diagnosed as schizophrenia per se. Schizophrenia and other psychotic disorders may be diagnosed using guidelines established in, for example, Diagnostic and Statistical Manual of Mental Disorders, Fourth Edition (DSM IV) Sections 293.81, 293.82, 295.10, 295.20, 295.30, 295.40, 295.60, 295.70, 295.90, 297.1, 297.3, 298.8.

The term “brain function” is used to describe the combined tasks of perceiving, integrating, filtering and responding to external stimuli and internal motivational processes.

The term “impaired” is used to describe a function working at a level that is less than normal. Impaired functions can be significantly impacted such that a function is barely being carried out, is virtually non-existent or is working in a fashion that is significantly less than normal. Impaired functions may also be sub-optimal. The impairment of function will vary in severity from patient to patient and the condition to be treated.

The term “respiratory depression” as used herein refers to a variety of conditions characterized by reduced respiratory frequency and inspiratory drive to cranial and spinal motor neurons. Specifically, respiratory depression refers to conditions where the medullary neural network associated with respiratory rhythm generating activity does not respond to accumulating levels of PCO₂ (or decreasing levels of PO₂) in the blood and subsequently under stimulates motor neurons controlling lung musculature.

The term “sleep apnea” as used herein refers to breathing-related sleep disorders of which there are two types: central and obstructive. Central Sleep Apnea is defined as a neurological condition causing cessation of all respiratory effort during sleep, usually with decreases in blood oxygen saturation, if the brainstem center controlling breathing shuts down there's no respiratory effort and no breathing. The person is aroused from sleep by an automatic breathing reflex, so may end up getting very little sleep at all. Obstructive sleep apnea is characterized by repetitive pauses in breathing during sleep due to the obstruction and/or collapse of the upper airway and followed by an awakening to breathe. Respiratory effort continues during the episodes of apnea.

The term “pro-drug” as used herein refers to a metabolically labile derivative that is pharmacologically inactive in the parent form but that is rapidly metabolized in human or animal plasma to a pharmacologically active form. Examples of pro-drugs as used herein include but in no way are limited to ester derivatives of hydroxyl containing moieties, such esters include but are not limited to those formed from substituted or un-substituted natural or un-natural amino acids.

Compounds of the Invention

The present invention is directed, in one aspect, to compounds having the property of enhancing RMPA receptor function. These are compounds having the structure of formula I, below:

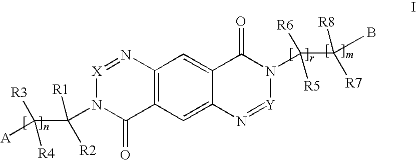

wherein:
X═C(C—H) or N,
Y═C(C—H) or N, with the proviso that Y cannot be carbon when X═N and the group

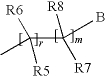

represents H, alkyl or cycloalkyl,
R¹ and R² are independently hydrogen, alkyl, substituted alkyl, cycloalkyl, substituted cycloalkyl, alkynyl, substituted alkynyl, alkenyl, substituted alkenyl, cyano, alkoxy, and if R¹ and R² are alkyl, R¹ and R² may be joined with a bond or —(CH₂)ₚ— to produce a cycloalkyl,
R³ and R⁴ are independently hydrogen, alkyl, substituted alkyl, cycloalkyl, substituted cycloalkyl, alkynyl, substituted alkynyl, alkenyl, substituted alkenyl, hydroxyl, alkoxy, cyano, fluoro,
A may be absent, hydrogen, alkyl, substituted alkyl, cycloalkyl, substituted cycloalkyl, alkynyl, substituted alkynyl, alkenyl, substituted alkenyl, aromatic, substituted aromatic, heteroaromatic, substituted heteroaromatic, bicycloheteroaromatic, heterocycle, substituted heterocycle, hydroxyl, alkoxy, cyano, fluoro, SCN, SO₂NR⁹R¹⁰, CONR⁹R¹⁰, NR¹¹SO₂R¹², NR¹¹COR¹², OR⁹, NR⁹R¹⁰,
R⁵ and R⁶ are independently hydrogen, alkyl, substituted alkyl, cycloalkyl, substituted cycloalkyl, alkynyl, substituted alkynyl, alkenyl, substituted alkenyl, cyano, alkoxy, and if R⁵ and R⁶ are alkyl, R⁵ and R⁶ may be joined with a bond or —(CH₂)ₚ— to produce a cycloalkyl,
R⁷ and R⁸ are independently hydrogen, allyl, substituted alkyl, cycloalkyl, substituted cycloalkyl, alkynyl, substituted alkynyl, alkenyl, substituted alkenyl, hydroxyl, alkoxy, cyano, fluoro,
R⁹, R¹⁰, R¹¹ and R¹², are independently hydrogen, alkyl, substituted alkyl, cycloalkyl, substituted cycloalkyl, alkynyl, substituted alkynyl, alkenyl, substituted alkenyl,
R⁹ and R¹⁰, and R¹¹ and R¹² may be joined with a bond or —(CH₂)_{q}— to produce a cycloalkyl,
B may be absent, hydrogen, alkyl, substituted alkyl, cycloalkyl, substituted cycloalkyl, alkynyl, substituted alkynyl, alkenyl, substituted alkenyl, aromatic, substituted aromatic, heteroaromatic, substituted heteroaromatic, bicycloheteroaromatic, heterocycle, substituted heterocycle, hydroxyl, alkoxy, cyano, fluoro, SCN, SO₂NR⁹R¹⁰, CONR⁹R¹⁹, NR¹¹SO₂R¹², NR¹¹COR¹², OR⁹, NR⁹R¹⁰,
n=0, 1 or 2,
m=0, 1 or 2,
p=1, 2, or 3,
q=2, 3 or 4
r=0 or 1, or a pharmaceutically acceptable salt, solvate or polymorph thereof.

Alternative preferred embodiments include compounds according to formula II below:

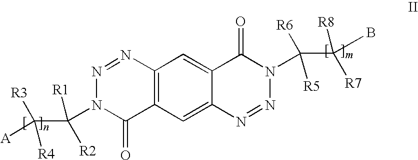

wherein:
R¹, R², R³, R⁴, R⁵, R⁶, A, B, n and m are as defined for formula I, or a pharmaceutically acceptable salt, solvate or polymorph thereof.

A further preferred embodiment includes compounds according to formula III below:

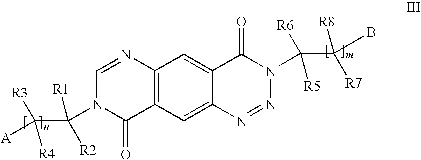

wherein:
R¹, R², R³, R⁴, R⁵, R⁶, A, B, n and m are as defined for formula I, or a pharmaceutically acceptable salt, solvate or polymorph thereof.

A further preferred embodiment includes compounds according to formula IV below:

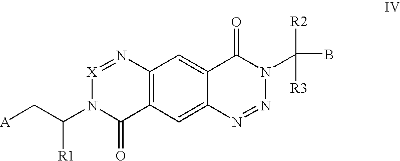

wherein:
X═C(C—H) or N,
R¹ is hydrogen, alkyl, substituted alkyl, cycloalkyl, substituted cycloalkyl, alkenyl, substituted alkenyl, alkynyl, substituted alkynyl,
R² and R³ are independently hydrogen, alkyl, substituted alkyl, cycloalkyl, substituted cycloalkyl, alkenyl, substituted alkenyl, alkynyl, substituted alkynyl, and R² and R³ may be joined with a bond or —(CH₂)ₚ— to produce a cycloalkyl, Particular preferred groups for A include but are not limited to aromatic, substituted aromatic, 5-ring heteroaromatics, substituted 5-ring heteroaromatics, 6-ring heteroaromatics, substituted 6-ring heteroaromatics,
p=1, 2 or 3,
B may be absent, hydrogen, alkyl, substituted alkyl, cycloalkyl, substituted cycloalkyl, alkynyl, substituted alkynyl, alkenyl, substituted alkenyl, aromatic, substituted aromatic, 5-ring heteroaromatics, substituted 5-ring heteroaromatics, 6-ring heteroaromatics, substituted 6-ring heteroaromatics, or a pharmaceutically acceptable salt, solvate or polymorph thereof.

A further preferred embodiment includes compounds according to formula V below:

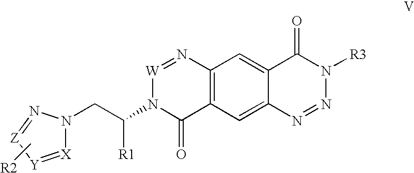

wherein:
W═C(C—H) or N,
X, Y and Z are independently C(C—H) or N to a maximum of 4 N's in the ring,
R¹ is hydrogen, methyl, ethyl, acetylene, cyclopropyl, fluoro,
R² is hydrogen, methyl, ethyl, CF₃,
R³ is methyl, ethyl, cyclopropyl, isopropyl, —(CH₂)ₚCCH, —(CH₂)ₚOR⁴, —(CH₂)ₚCN,
R⁴ is H, alkyl, substituted alkyl, cycloalkyl, substituted cycloalkyl, alkynyl, substituted alkynyl, alkenyl, substituted alkenyl, aromatic, substituted aromatic,
p=0, 1, 2 or 3, or a pharmaceutically acceptable salt, solvate or polymorph thereof.

A further preferred embodiment includes compounds according to formula VI below:

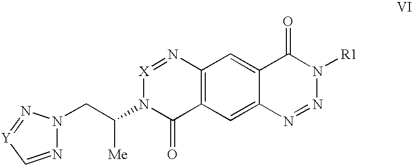

wherein:
X═C(C—H) or N,
Y═C(C—H) or N,
R¹ is methyl, ethyl, cyclopropyl, isopropyl, —(CH₂)ₚCCH, —(CH₂)ₚOR², —(CH₂)ₚCN,
R² is H, alkyl, substituted alkyl, cycloalkyl, substituted cycloalkyl, alkynyl, substituted alkynyl, alkenyl, substituted alkenyl, aromatic, substituted aromatic,
p=0, 1, 2 or 3, or a pharmaceutically acceptable salt, solvate or polymorph thereof.

A yet further preferred embodiment includes compounds according to formula VII below:

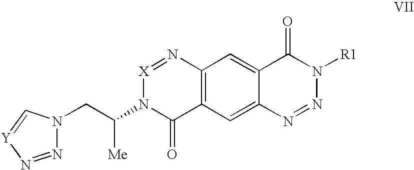

wherein:
X═C(C—H) or N,
Y═C(C—H) or N,
R¹ is methyl, ethyl, cyclopropyl, isopropyl, —(CH₂)ₚCCH, —(CH₂)ₚOR², —(CH₂)ₚCN,
R² is H, alkyl, substituted alkyl, cycloalkyl, substituted cycloalkyl, alkynyl, substituted alkynyl, alkenyl, substituted alkenyl, aromatic, substituted aromatic,
p=0, 1, 2 or 3, or a pharmaceutically acceptable salt, solvate or polymorph thereof.

A yet further preferred embodiment includes compounds according to formula VIII below:

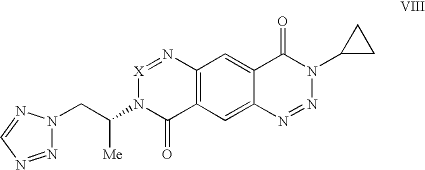

wherein:
X═C(C—H) or N, or a pharmaceutically acceptable salt, solvate or polymorph thereof.

A yet further preferred embodiment includes compounds according to formula IX below:

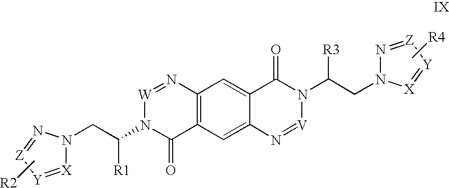

wherein:
V and W are independently C(C—H) or N,
X, Y and Z are independently C(C—H) or N to a maximum of 4 N's in the ring,
R¹ and R3 are independently hydrogen, methyl, ethyl, acetylene, cyclopropyl, fluoro,
R² and R4 are independently hydrogen, methyl, ethyl, CF₃, or a pharmaceutically acceptable salt, solvate or polymorph thereof.

A further preferred class of compounds is shown in formula X below:

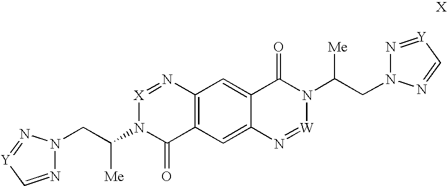

wherein:
X and W are independently C (C—H) or N,
Y═C(C—H) or N, or a pharmaceutically acceptable salt, solvate or polymorph thereof.

A further preferred class of compounds is shown in formula XI below:

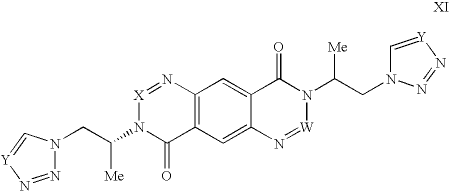

wherein:
X and W are independently C or N,
Y═C or N, or a pharmaceutically acceptable salt, solvate or polymorph thereof.

In a further aspect, the present invention provides compounds of Formulas I-XI selected from:
3-Cyclopropyl-8-[(1R)-1-methyl-2-(2H-tetrazol-2-yl)ethyl]-3,8-dihydro[1,2,3]triazino[4,5-g][1,2,3]benzotriazine-4,9-dione
3-Cyclopropyl-8-[(2R)-1-(5-methyl-2H-tetrazol-2-yl)propan-2-yl]-3,8-dihydrobenzo[1,2-d:4,5-d′]bis[1,2,3]triazine-4,9-dione
3-Cyclopropyl-8-[(2R)-1-(5-methyl-1H-tetrazol-1-yl)propan-2-yl]-3,8-dihydrobenzo[1,2-d:4,5-d′]bis[1,2,3]triazine-4,9-dione
3-Methyl-8-[(2R)-1-(2H-tetrazol-2-yl)propan-2-yl]-3,8-dihydrobenzo[1,2-d:4,5-d′]bis[1,2,3]triazine-4,9-dione
3-Methyl-8-[(2R)-1-(1H-tetrazol-1-yl)propan-2-yl]-3,8-dihydrobenzo[1,2-d:4,5-d′]bis[1,2,3]triazine-4,9-dione
3-Ethyl-8-[(2R)-1-(2H-tetrazol-2-yl)propan-2-yl]-3,8-dihydrobenzo[1,2-d:4,5-d′]bis[1,2,3]triazine-4,9-dione
3-(2-Fluoroethyl)-8-[(2R)-1-(2H-tetrazol-2-yl)propan-2-yl]-3,8-dihydrobenzo[1,2-d:4,5-d′]bis[1,2,3]triazine-4,9-dione
3-Propan-2-yl-8-[(2R)-1-(2H-tetrazol-2-yl)propan-2-yl]-3,8-dihydrobenzo[1,2-d:4,5-d′]bis[1,2,3]triazine-4,9-dione
3-Cyclobutyl-8-[(2R)-1-(2H-tetrazol-2-yl)propan-2-yl]-3,8-dihydrobenzo[1,2-d:4,5-d′]bis[1,2,3]triazine-4,9-dione
3-(Cyclopropylmethyl)-8-[(2R)-1-(2H-tetrazol-2-yl)propan-2-yl]-3,8-dihydrobenzo[1,2-d:4,5-d′]bis[1,2,3]triazine-4,9-dione
3-(2-Methylpropyl)-8-[(2R)-1-(2H-tetrazol-2-yl)propan-2-yl]-3,8-dihydrobenzo[1,2-d:4,5-d′]bis[1,2,3]triazine-4,9-dione
3-But-3-yn-1-yl-8-[(2R)-1-(2H-tetrazol-2-yl)propan-2-yl]-3,8-dihydrobenzo[1,2-d:4,5-d′]bis[1,2,3]triazine-4,9-dione
3-But-3-yn-1-yl-8-[(2R)-1-(1H-tetrazol-1-yl)propan-2-yl]-3,8-dihydrobenzo[1,2-d:4,5-d′]bis[1,2,3]triazine-4,9-dione
3-Cyclopropyl-8-(1-pyridin-3-ylpropan-2-yl)-3,8-dihydrobenzo[1,2-d: 4,5-d′]bis[1,2,3]triazine-4,9-dione
3-Cyclopropyl-8-[2-(2H-tetrazol-2-yl)ethyl]-3,8-dihydrobenzo[1,2-d:4,5-d′]bis[1,2,3]triazine-4,9-dione
3-Cyclopropyl-8-[(2R)-1-(1H-tetrazol-1-yl)propan-2-yl]-3,8-dihydrobenzo[1,2-d:4,5-d′]bis[1,2,3]triazine-4,9-dione
3-Cyclopropyl-8-[(2R)-1-(2H-tetrazol-2-yl)propan-2-yl]-3,8-dihydropyrimido[4,5-g]quinazoline-4,9-dione
3-(2-Methoxyethyl)-8-[(2R)-1-(2H-tetrazol-2-yl)propan-2-yl]-3,8-dihydrobenzo[1,2-d:4,5-d′]bis[1,2,3]triazine-4,9-dione
3-(2-Methoxyethyl)-8-[(2R)-1-(2H-tetrazol-2-yl)propan-2-yl]-3,8-dihydro[1,2,3]triazino[4,5-g]quinazoline-4,9-dione
3-(Pyridin-3-ylmethyl)-8-[(2R)-1-(2H-tetrazol-2-yl)propan-2-yl]-3,8-dihydrobenzo[1,2-d:4,5-d′]bis[1,2,3]triazine-4,9-dione
3-Prop-2-yn-1-yl-8-[(2R)-1-(2H-tetrazol-2-yl)propan-2-yl]-3,8-dihydrobenzo[1,2-d:4,5-d′]bis[1,2,3]triazine-4,9-dione
{4,9-Dioxo-8-[(2R)-1-(2H-tetrazol-2-yl)propan-2-yl]-8,9-dihydrobenzo[1,2-d:4,5-d′]bis[1,2,3]triazin-3(4H)-yl}acetonitrile
3-Cyclopropyl-8-[(2R)-1-(2H-tetrazol-2-yl)but-3-yn-2-yl]-3,8-dihydrobenzo[1,2-d:4,5-d′]bis[1,2,3]triazine-4,9-dione
3-Cyclopropyl-8-[(2R)-1-(1H-tetrazol-1-yl)but-3-yn-2-yl]-3,8-dihydrobenzo[1,2-d:4,5-d′]bis[1,2,3]triazine-4,9-dione
3-Cyclopropyl-8-[(2R)-1-(2H-1,2,3-triazol-2-yl)propan-2-yl]-3,8-dihydrobenzo[1,2-d:4,5-d′]bis[1,2,3]triazine-4,9-dione
3-Cyclopropyl-8-[(2R)-1-(1H-1,2,3-triazol-1-yl)propan-2-yl]-3,8-dihydrobenzo[1,2-d:4,5-d′]bis[1,2,3]triazine-4,9-dione
3-Methyl-8-[(2R)-1-(2H-1,2,3-triazol-2-yl)propan-2-yl]-3,8-dihydrobenzo[1,2-d:4,5-d′]bis[1,2,3]triazine-4,9-dione
3-Methyl-8-[(2R)-1-(1H-1,2,3-triazol-1-yl)propan-2-yl]-3,8-dihydrobenzo[1,2-d:4,5-d′]bis[1,2,3]triazine-4,9-dione
3-(2-Methoxyethyl)-8-[(2R)-1-(2H-1,2,3-triazol-2-yl)propan-2-yl]-3,8-dihydrobenzo[1,2-d:4,5-d′]bis[1,2,3]triazine-4,9-dione
3-Cyclopropyl-8-[(2R)-1-(1H-1,2,4-triazol-1-yl)propan-2-yl]-3,8-dihydrobenzo[1,2-d:4,5-d′]bis[1,2,3]triazine-4,9-dione
3-Cyclopropyl-8-[1-(1H-pyrazol-1-yl)propan-2-yl]-3,8-dihydrobenzo[1,2-d:4,5-d′]bis[1,2,3]triazine-4,9-dione
3-Methyl-8-[1-(1H-pyrazol-1-yl)propan-2-yl]-3,8-dihydrobenzo[1,2-d:4,5-d′]bis[1,2,3]triazine-4,9-dione
(2R)-2-(8-Cyclopropyl-4,9-dioxo-8,9-dihydrobenzo[1,2-d:4,5-d′]bis[1,2,3]triazin-3(4H)-yl)propyl thiocyanate
3-But-2-yn-1-yl-8-[(2R)-1-(2H-tetrazol-2-yl)propan-2-yl]-3,8-dihydrobenzo[1,2-d: 4,5-d′]bis[1,2,3]triazine-4,9-dione
N-[(2R)-2-(8-Cyclopropyl-4,9-dioxo-8,9-dihydrobenzo[1,2-d:4,5-d′]bis[1,2,3]triazin-3(4H)-yl)propyl]methanesulfonamide
N-[(2R)-2-(8-Cyclopropyl-4,9-dioxo-8,9-dihydrobenzo[1,2-d:4,5-d′]bis[1,2,3]triazin-3(4H)-yl)propyl]-N-methylmethanesulfonamide
3-Cyclopropyl-8-[(2R)-1-(3-fluorophenyl)but-3-yn-2-yl]-3,8-dihydrobenzo[1,2-d: 4,5-d′]bis[1,2,3]triazine-4,9-dione
3-[(2R)-1-(2H-Tetrazol-2-yl)propan-2-yl]-8-[(2R)-1-(2H-1,2,3-triazol-2-yl)propan-2-yl]-3,8-dihydrobenzo[1,2-d:4,5-d′]bis[1,2,3]triazine-4,9-dione
3-[(2R)-1-(2H-Tetrazol-2-yl)propan-2-yl]-8-[(2R)-1-(1H-1,2,3-triazol-1-yl)propan-2-yl]-3,8-dihydrobenzo[1,2-d:4,5-d′]bis[1,2,3]triazine-4,9-dione
3-[(2R)-1-(2H-Tetrazol-2-yl)propan-2-yl]-8-[(2R)-1-(2H-tetrazol-2-yl)propan-2-yl]-3,8-dihydrobenzo[1,2-d:4,5-d′]bis[1,2,3]triazine-4,9-dione
3-[(2R)-1-(2H-Tetrazol-2-yl)propan-2-yl]-8-[(2S)-1-(1H-tetrazol-1-yl)propan-2-yl]-3,8-dihydrobenzo[1,2-d:4,5-d′]bis[1,2,3]triazine-4,9-dione
3-Methoxy-8-[(2R)-1-(2H-tetrazol-2-yl)propan-2-yl]-3,8-dihydrobenzo[1,2-d:4,5-d′]bis[1,2,3]triazine-4,9-dione
3-(3-Methoxypropyl)-8-[(2R)-1-(2H-tetrazol-2-yl)propan-2-yl]-3,8-dihydrobenzo[1,2-d: 4,5-d′]bis[1,2,3]triazine-4,9-dione
3-Prop-2-en-1-yl-8-[(2R)-1-(2H-tetrazol-2-yl)propan-2-yl]-3,8-dihydrobenzo[1,2-d:4,5-d′]bis[1,2,3]triazine-4,9-dione
(3R)-3-(8-Cyclopropyl-4,9-dioxo-8,9-dihydrobenzo[1,2-d:4,5-d′]bis[1,2,3]triazin-3(4H)-yl)butanenitrile
(3R)-3-{4,9-Dioxo-8-[(2R)-1-(2H-1,2,3-triazol-2-yl)propan-2-yl]-8,9-dihydrobenzo[1,2-d:4,5-d′]bis[1,2,3]triazin-3(4H)-yl}butanenitrile
(3R)-3-{4,9-Dioxo-8-[(2R)-1-(2H-tetrazol-2-yl)propan-2-yl]-8,9-dihydrobenzo[1,2-d:4,5-d′]bis[1,2,3]triazin-3(4H)-yl}butanenitrile
3-But-2-yn-1-yl-8-[(2R)-1-(2H-tetrazol-2-yl)propan-2-yl]-3,8-dihydrobenzo[1,2-d:4,5-d′]bis[1,2,3]triazine-4,9-dione
3-But-2-yn-1-yl-8-[(2R)-1-(2H-1,2,3-triazol-2-yl)propan-2-yl]-3,8-dihydrobenzo[1,2-d:4,5-d′]bis[1,2,3]triazine-4,9-dione
3-Pent-3-yn-2-yl-8-[(2R)-1-(2H-tetrazol-2-yl)propan-2-yl]-3,8-dihydrobenzo[1,2-d:4,5-d′]bis[1,2,3]triazine-4,9-dione
3,8-Bis[(2R)-1-(2H-1,2,3-triazol-2-yl)propan-2-yl]-3,8-dihydrobenzo[1,2-d:4,5-d′]bis[1,2,3]triazine-4,9-dione
3,8-Bis[(2R)-1-(1H-1,2,3-triazol-1-yl)propan-2-yl]-3,8-dihydrobenzo[1,2-d:4,5-d′]bis[1,2,3]triazine-4,9-dione
3-Cyclopropyl-8-[(2R)-1-(2H-tetrazol-2-yl)propan-2-yl]-3,8-dihydro[1,2,3]triazino[4,5-g]quinazoline-4,9-dione
3-Cyclopropyl-8-[(2R)-1-(1H-tetrazol-1-yl)propan-2-yl]-3,8-dihydro[1,2,3]triazino[4,5-g]quinazoline-4,9-dione
8-[(2R)-1-(1H-Benzotriazol-1-yl)propan-2-yl]-3-cyclopropyl-3,8-dihydro[1,2,3]triazino[4,5-g]quinazoline-4,9-dione
3-Cyclopropyl-8-[(2R)-1-(2H-tetrazol-2-yl)butan-2-yl]-3,8-dihydro[1,2,3]triazino[4,5-g]quinazoline-4,9-dione
3-Cyclopropyl-8-[(2R)-1-(1H-tetrazol-1-yl)butan-2-yl]-3,8-dihydro[1,2,3]triazino[4,5-g]quinazoline-4,9-dione
3-Cyclopropyl-8-[(2R)-1-(2H-1,2,3-triazol-2-yl)butan-2-yl]-3,8-dihydro[1,2,3]triazino[4,5-g]quinazoline-4,9-dione
3-Cyclopropyl-8-[(2R)-1-(1H-1,2,3-triazol-1-yl)butan-2-yl]-3,8-dihydro[1,2,3]triazino[4,5-g]quinazoline-4,9-dione
3-Prop-2-yn-1-yl-8-[(2R)-1-(2H-tetrazol-2-yl)propan-2-yl]-3,8dihydro[1,2,3]triazino[4,5-g]quinazoline-4,9-dione
3-Prop-2-yn-1-yl-8-[(2R)-1-(1H-tetrazol-1-yl)propan-2-yl]-3,8-dihydro[1,2,3]triazino[4,5-g]quinazoline-4,9-dione
8-[(2R)-1-(2H-Tetrazol-2-yl)propan-2-yl]-3-(1H-1,2,3-triazol-4-ylmethyl)-3,8-dihydro[1,2,3]triazino[4,5-g]quinazoline-4,9-dione
3-Cyclopropyl-8-[2-(2H-tetrazol-2-yl)ethyl]-3,8-dihydro[1,2,3]triazino[4,5-g]quinazoline-4,9-dione
3,8-Bis[(2R)-1-(2H-tetrazol-2-yl)propan-2-yl]-3,8-dihydro[1,2,3]triazino[4,5g]quinazoline-4,9-dione
3-[(2R)-1-(1H-Tetrazol-1-yl)propan-2-yl]-8-[(2R)-1-(2H-tetrazol-2-yl)propan-2-yl]-3,8-dihydro[1,2,3]triazino[4,5-g]quinazoline-4,9-dione
8-[(2R)-1-(2H-Tetrazol-2-yl)butan-2-yl]-3-[(2R)-1-(2H-tetrazol-2-yl)propan-2-yl]-3,8-dihydro[1,2,3]triazino[4,5-g]quinazoline-4,9-dione
8-[(2R)-1-(1H-Tetrazol-1-yl)butan-2-yl]-3-[(2R)-1-(2H-tetrazol-2-yl)propan-2-yl]-3,8-dihydro[1,2,3]triazino[4,5-g]quinazoline-4,9-dione
8-[(2R)-1-(5-Methyl-2H-tetrazol-2-yl)butan-2-yl]-3-[(2R)-1-(2H-tetrazol-2-yl)propan-2-yl]-3,8-dihydro[1,2,3]triazino[4,5-g]quinazoline-4,9-dione
8-[(2R)-1-(5-Methyl-1H-tetrazol-1-yl)butan-2-yl]-3-[(2R)-1-(2H-tetrazol-2-yl)propan-2-yl]-3,8dihydro[1,2,3]triazino[4,5-g]quinazoline-4,9-dione
3,8-Bis[(2R)-1-(2H-tetrazol-2-yl)butan-2-yl]-3,8-dihydro[1,2,3]triazino[4,5-g]quinazoline-4,9-dione
3,8-Bis[(2R)-1-(1H-tetrazol-1-yl)butan-2-yl]-3,8dihydro[1,2,3]triazino[4,5-g]quinazoline-4,9-dione
3,8-Bis[(2R)-1-(2H-tetrazol-2-yl)butan-2-yl]-3,8-dihydropyrimido[4,5-g]quinazoline-4,9-dione
3-[(2R)-1-Hydroxybutan-2-yl]-8-[(2R)-1-(2H-tetrazol-2-yl)butan-2-yl]-3,8-dihydropyrimido[4,5-g]quinazoline-4,9-dione
3-[(2R)-1-(2H-Tetrazol-2-yl)butan-2-yl]-8-[(2R)-1-(4H-1,2,4-triazol-4-yl)butan-2-yl]-3,8-dihydropyrimido[4,5-g]quinazoline-4,9-dione
3,8-Bis[(2R)-1-(2H-tetrazol-2-yl)propan-2-yl]-3,8-dihydropyrimido[4,5-g]quinazoline-4,9-dione
3,8-Bis[(2R)-1-(1H-pyrazol-1-yl)propan-2-yl]-3,8-dihydropyrimido[4,5-g]quinazoline-4,9-dione
3-[(2R)-1-(4-Chloro-1H-pyrazol-1-yl)propan-2-yl]-8-[(2R)-1-(1H-pyrazol-1-yl)propan-2-yl]-3,8-dihydropyrimido[4,5-g]quinazoline-4,9-dione
3,8-Bis[2-(3-fluorophenyl)ethyl]-3,8-dihydropyrimido[4,5-g]quinazoline-4,9-dione
3-[(2R)-1-Hydroxypropan-2-yl]-8-[(2R)-1-(2H-tetrazol-2-yl)propan-2-yl]-3,8-dihydrobenzo[1,2-d:4,5-d′]bis[1,2,3]triazine-4,9-dione
3,8-Bis[(2R)-1-(2H-tetrazol-2-yl)propan-2-yl]-3,8-dihydrobenzo[1,2-d:4,5-d′]bis[1,2,3]triazine-4,9-dione
3-[(2R)-1-(5-Methyl-2H-tetrazol-2-yl)propan-2-yl]-8-[(2R)-1-(2H-tetrazol-2-yl)propan-2-yl]-3,8-dihydrobenzo[1,2-d:4,5-d′]bis[1,2,3]triazine-4,9-dione
3-[(2R)-1-(5-Methyl-1H-tetrazol-1-yl)propan-2-yl]-8-[(2R)-1-(2H-tetrazol-2-yl)propan-2-yl]-3,8-dihydrobenzo[1,2-d:4,5-d′]bis[1,2,3]triazine-4,9-dione
3-[(2R)-1-(2H-Tetrazol-2-yl)propan-2-yl]-8-{(2R)-1-[3-(trifluoromethyl)-1H-pyrazol-1-yl]propan-2-yl}-3,8-dihydrobenzo[1,2-d:4,5-d′]bis[1,2,3]triazine-4,9-dione
3-[(2R)-1-(2H-Tetrazol-2-yl)butan-2-yl]-8-[(2R)-1-(2H-tetrazol-2-yl)propan-2-yl]-3,8-dihydrobenzo[1,2-d:4,5-d′]bis[1,2,3]triazine-4,9-dione
3-[(2R)-1-(1H-Tetrazol-1-yl)butan-2-yl]-8-[(2R)-1-(2H-tetrazol-2-yl)propan-2-yl]-3,8-dihydrobenzo[1,2-d:4,5-d′]bis[1,2,3]triazine-4,9-dione
3-[(2R)-1-(2H-Tetrazol-2-yl)propan-2-yl]-8-[(2R)-1-(2H-1,2,3-triazol-2-yl)butan-2-yl]-3,8-dihydrobenzo[1,2-d:4,5-d′]bis[1,2,3]triazine-4,9-dione
3-[(2R)-1-(1H-Tetrazol-1-yl)propan-2-yl]-8-[(2R)-1-(2H-tetrazol-2-yl)propan-2-yl]-3,8-dihydrobenzo[1,2-d:4,5-d′]bis[1,2,3]triazine-4,9-dione
3,8-Bis[(2R)-1-(1H-tetrazol-1-yl)propan-2-yl]-3,8-dihydrobenzo[1,2-d:4,5-d′]bis[1,2,3]triazine-4,9-dione
3-(2-Hydroxy-2-methylpropyl)-8-[(2R)-1-(2H-tetrazol-2-yl)propan-2-yl]-3,8-dihydrobenzo[1,2-d:4,5-d′]bis[1,2,3]triazine-4,9-dione

Synthesis

The synthesis of the compounds of the invention, are preferably carried out as shown in the following Schemes I and II. Alternative syntheses by analogy relying on methodology that exists in the art may also be used.

The 4-nitroaniline derivative 4 is synthesized in 2 steps starting with 2-aminoterephthalic acid methylester 1 by firstly (step A) protecting the aniline using for example a mixture of formic acid and acetic acid anhydride or ethyl chloroformate in the presence of a base e.g. N-methyl morpholine, or triethylamine to give intermediate 2 and then nitrating (step B) with nitric acid/sulfuric acid which results in a mixture of nitration products, compounds 3 and 4 that can be readily separated by silica gel chromatography or crystallization. The desired nitration product 4, which was identified as the less polar isomer on silica gel, was reduced (step C) to the aniline using Zn/Cu and acid (HCOOH, CH₃COOH, HCl or others) or hydrogen and a catalyst (Pd/C or others). Diazotation of this aniline 5 with NaNO₂/HCl and treatment with an amine under basic conditions (step D) yielded the triazinones 6. Hydrolysis of the aniline and ester functionality under basic conditions (NaOH, KOH or others) and amide formation, using standard conditions for example CDI, EDCI, HBTU in a suitable solvent yielded the desired amides. The ring closure (step F) to the mixed tricyclic triazinones/quinazolinones 8 can be achieved using an orthoformate and an acidic catalyst such as toluene sulfonic acid at elevated temperature. Alternatively the tricyclic bis-triazinone system 9 can be formed (step G), when the amide is treated with NaNO₂ or an organic nitrite (isoamyl nitrite, tert butyl nitrite or others) under acidic conditions (HCl, CH₃COOH or others). An alternative route to the tricyclic system 9 starts with the hydrolysis of nitro aniline derivative 4 under basic conditions (NaOH, KOH or others) followed by amide formation under standard conditions for example CDI, EDCI, HBTU in a suitable solvent (step E), followed by reduction (step H) of the formed nitro derivative 7 by using Zn/Cu and acid (e.g. HCOOH, CH₃COOH, HCl) or hydrogen and a catalyst (Pd/C or others). Ring closure to the bis-triazinone (step H) can be performed with NaNO₂ or an organic nitrite (e.g. isoamyl nitrite, tert butyl nitrite) under acidic conditions (e.g. HCl, CH₃COOH) to yield 9.

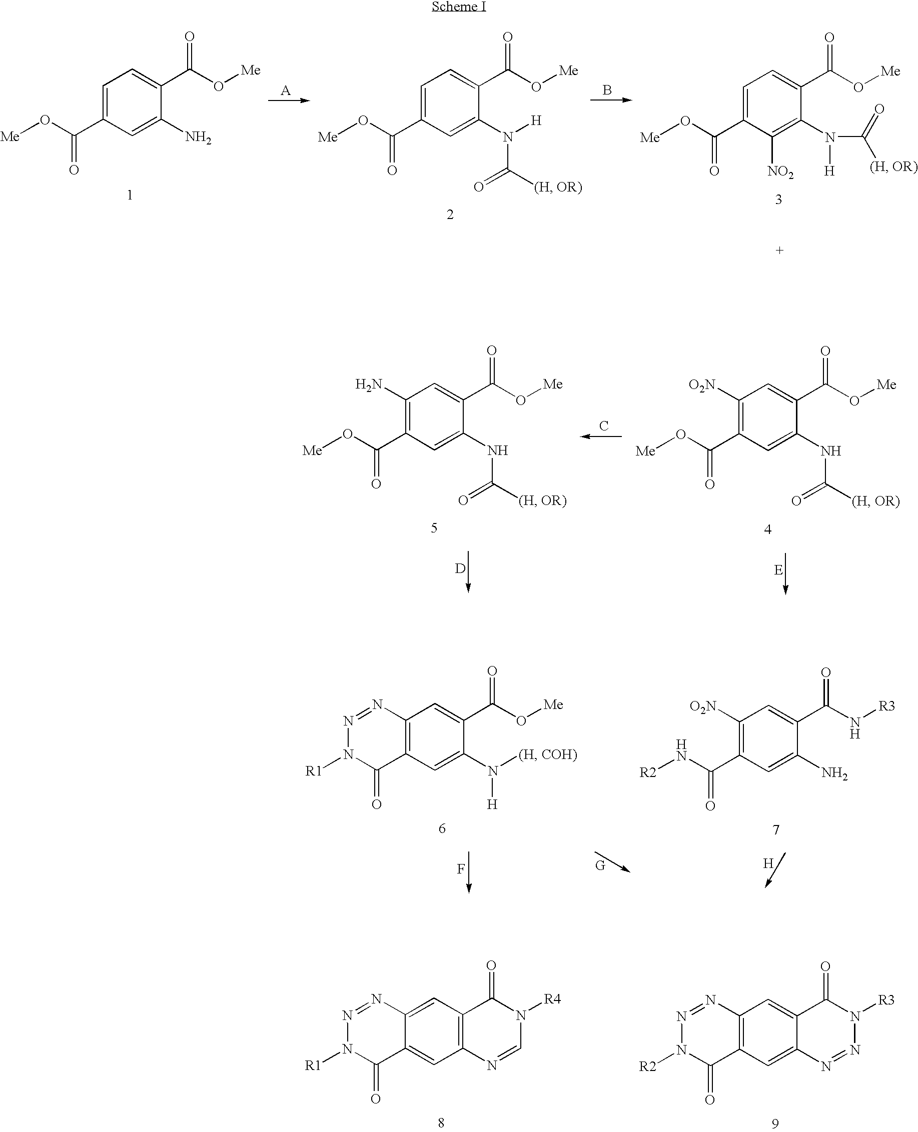

The amides 5 (Scheme II) can be transformed into quinazolinones 10 by heating with substituted amines (H₂N—R₁, H₂N—R₂) in a suitable solvent (step K). Hydrolysis of the aniline and ester functionality under basic conditions (NaOH, KOH or others) followed by amide formation, using standard conditions for example CDI, EDCI, HBTU, in a suitable solvent yielded amides which were used without purification in the next step (N). Ring closure to the quinazolinone-triazinones (step N) can be performed with NaNO₂ or an organic nitrite (e.g. isoamyl nitrite, tert butyl nitrite or others) under acidic conditions (e.g. HCl, CH₃COOH or others) to yield 13. The ring closure (step O) to the bis-quinazolinone system 14 can be achieved using an orthoformate and an acidic catalyst such as toluene sulfonic acid (or others) at elevated temperature. An alternative, especially when symmetrical bis-quinazolinones are synthesized is the formation of the bis-formamide 11 using formic acid and acetic acid anhydride at elevated temperatures (step L). Heating 11 with substituted amines (step P) gives bis-quinazolinones 14. The synthesis of the tricyclic system 14 starts with the hydrolysis of nitro aniline derivative 4 under basic conditions (e.g. NaOH, KOH) followed by amide formation under standard conditions for example CDI, EDCI, HBTU in a suitable solvent (step M), followed by reduction (step R) of the formed nitro derivative 12 by using Zn/Cu and acid (e.g. HCOOH, CH₃COOH, HCl) or hydrogen and a catalyst (e.g. Pd/C). Ring closure to the bis-quinazolinone 14 (step R) can be achieved using an orthoformate and an acidic catalyst such as toluene sulfonic acid (or others) at elevated temperature.

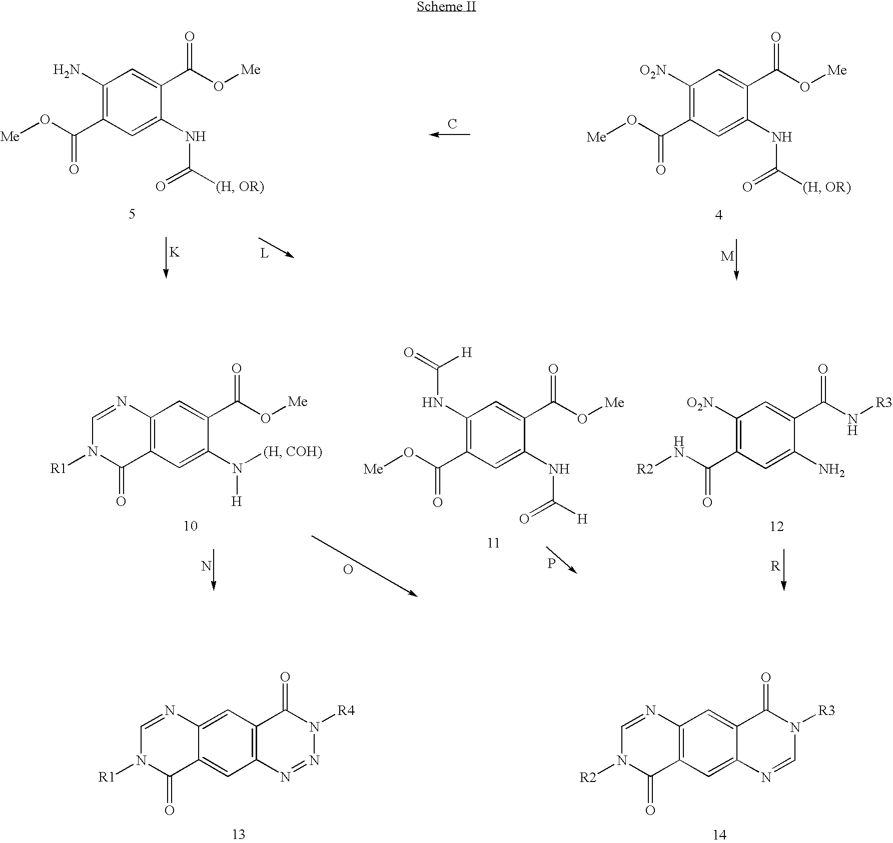

The synthesis of the compounds of the invention, are preferably carried out as shown in Schemes. Alternative syntheses by analogy relying on methodology that exists in the art may also be used.

Method of Treatment

According to one aspect of the invention, a method is provided for treating a mammalian subject suffering from a hypoglutamatergic condition, or from deficiencies in the number or strength of excitatory synapses or in the number of AMPA receptors. In such a subject, memory or other cognitive functions may be impaired, or cortical/striatal imbalance may occur, leading to loss of memory, dementia, depression, attention disorders, sexual dysfunction, movement disorders, schizophrenia or schizophreniform behavior. Memory disorders and learning disorders, which are treatable according to the present invention include those disorders that result from aging, trauma, stroke and neurodegenerative disorders. Examples of neurodegenerative disorders include, but are not limited to, those associated with drug-induced states, neurotoxic agents, Alzheimer's disease, and aging. These conditions are readily recognized and diagnosed by those of ordinary skill in the art and treated by administering to the patient an effective amount of one or more compounds according to the present invention.

In another aspect, the invention provides a method for reducing or inhibiting respiratory depression in a subject having such a condition, comprising administering to the subject an amount of a compound of the invention, the amount being sufficient to reduce or inhibit respiratory depression. In a further aspect of the invention, a method is provided for reducing or inhibiting respiratory depression comprising administering to the subject an amount of a compound of the invention in combination with an opiate; examples of such opiates include but are not limited to, alfentanil and fentanyl.

In a further aspect, the invention provides a method for reducing or inhibiting breathing-related sleep disorders or sleep apnea in a subject having sleep apnea, comprising administering to the subject an amount of a compound of the invention, the amount being sufficient to reduce or inhibit the breathing related sleep disorder.

In the present invention, the method of treatment comprises administering to the subject in need of treatment, in a pharmaceutically acceptable carrier, an effective amount of a compound having the Formula of I below:

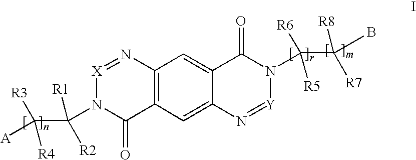

wherein:
X═C or N,
Y═C or N, with the exception that Y cannot be carbon when X═N and the group

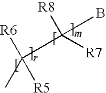

represents H, alkyl or cycloalkyl,
R¹ and R² are independently hydrogen, alkyl, substituted alkyl, cycloalkyl, substituted cycloalkyl, alkynyl, substituted alkynyl, alkenyl, substituted alkenyl, cyano, alkoxy, and if R¹ and R² are alkyl, R¹ and R² may be joined with a bond or —(CH₂)ₚ— to produce a cycloalkyl,
R³ and R⁴ are independently hydrogen, alkyl, substituted alkyl, cycloalkyl, substituted cycloalkyl, alkynyl, substituted alkynyl, alkenyl, substituted alkenyl, hydroxyl, alkoxy, cyano, fluoro,
A may be absent, hydrogen, alkyl, substituted alkyl, cycloalkyl, substituted cycloalkyl, alkynyl, substituted alkynyl, alkenyl, substituted alkenyl, aromatic, substituted aromatic, heteroaromatic, substituted heteroaromatic, bicycloheteroaromatic, heterocycle, substituted heterocycle, hydroxyl, alkoxy, cyano, fluoro, SCN, SO₂NR⁹R¹⁰, CONR⁹R¹⁰, NR¹¹SO₂R¹², NR¹¹COR¹², OR⁹, NR⁹R¹⁰,
R⁵ and R⁶ are independently hydrogen, alkyl, substituted alkyl, cycloalkyl, substituted cycloalkyl, alkynyl, substituted alkynyl, alkenyl, substituted alkenyl, cyano, alkoxy, and if R⁵ and R⁶ are alkyl, R⁵ and R⁶ may be joined with a bond or —(CH₂)ₚ— to produce a cycloalkyl,
R⁷ and R⁸ are independently hydrogen, alkyl, substituted alkyl, cycloalkyl, substituted cycloalkyl, alkynyl, substituted alkynyl, alkenyl, substituted alkenyl, hydroxyl, alkoxy, cyano, fluoro,
R⁹, R¹⁰, R¹¹ and R¹², are independently hydrogen, alkyl, substituted alkyl, cycloalkyl, substituted cycloalkyl, alkynyl, substituted alkynyl, alkenyl, substituted alkenyl,
R⁹ and R¹⁰ and R¹¹ and R¹² may be joined with a bond or —(CH₂)_{q}— to produce a cycloalkyl,
B may be absent, hydrogen, alkyl, substituted alkyl, cycloalkyl, substituted cycloalkyl, alkynyl, substituted alkynyl, alkenyl, substituted alkenyl, aromatic, substituted aromatic, heteroaromatic, substituted heteroaromatic, bicycloheteroaromatic, heterocycle, substituted heterocycle, hydroxyl, alkoxy, cyano, fluoro, SCN, SO₂NR⁹R¹⁰, CONR⁹R¹⁰, NR¹¹SO₂R¹², NR¹¹COR¹², OR⁹, NR⁹R¹⁰,
n=0, 1 or 2,
m=0, 1 or 2,
p=1, 2, or 3,
q=2, 3 or 4
r=0 or 1, or a pharmaceutically acceptable salt, solvate or polymorph thereof.

In the present invention, the method of treatment also comprises administering to the subject in need of treatment, in a pharmaceutically acceptable carrier, an effective amount of a compound having the Formulas II-XI as previously defined.

Compounds according to the present invention exhibit enhanced bioavailability in most instances due, at least in part, to enhanced pharmacokinetics exhibited by the present compounds. Accordingly, the present compounds may be favorably formulated into pharmaceutical compositions in a variety of dosage forms, and in particular, oral dosage forms.

As noted above, treatment of a subject according to the method of the invention is useful for enhancing AMPA receptor activity, and thus may be used to facilitate the learning of behaviors dependent upon AMPA receptors, and to treat conditions, such as memory impairment, in which AMPA receptors, or synapses utilizing these receptors, are reduced in numbers or efficiency. The method is also useful for enhancing excitatory synaptic activity in order to restore an imbalance between brain sub-regions, which may manifest itself in schizophrenia or schizophreniform behavior, or other behavior as described above. The compounds administered in accordance with the method have been found to be more effective than previously described compounds in enhancing AMPA receptor activity, as shown in the in vivo tests described below.

Biological Activity: Enhancement of AMPA Receptor Function

I. In Vitro Assays

Synaptic responses mediated by AMPA receptors are increased according to the method of the invention, using the compounds described herein. These compounds are demonstrated, in the Examples that follow, to have potent activity in increasing AMPA mediated whole cell currents in cultured neurons and AMPA receptor function in slices of rat hippocampus. The physiological effects of invention compounds were tested in vitro on primary cultures of rat cortical or hippocampal neurons or on slices of rat hippocampus according to the following procedures.

Patch Clamp Electrophysiology Assay

Cortical cells were prepared from day 18-19 embryonic Sprague-Dawley rats and recorded after 3 days in culture. The extracellular solution (ECS) contained (in mM): NaCl (145), KCl (5.4), HEPES (10), MgCl2 (0.8), CaCl2 (1.8), glucose (10), sucrose (30); pH. 7.4. In order to block the voltage-gated sodium currents, 40 nM TTX was added to the recording solution. The intracellular solution contained (in mM): K-gluconate (140), HEPES (20), EGTA (1.1), phosphocreatine (5), MgATP (3), GTP (0.3), MgCl2 (5), and CaCl2 (0.1); pH: 7.2. All test compound and glutamate solutions were made-up in the extracellular solution.

The whole-cell current was measured with patch-clamp amplifier (Axopatch 200B), filtered at 2 kHz, digitized at 5 kHz and recorded on a PC with pClamp 8. The cells were voltage-clamped at −80 mV. Solutions were applied by DAD-12 system. A baseline response for each cell was recorded using a 1 s pulse of 500 μM glutamate dissolved in ECS. Responses to test compound were then determined by application of a 10 s pulse of test compound followed by a 1 s pulse of the same concentration of test compound plus 500 μM glutamate and then 10 s of saline. This pulse sequence was repeated until a stable reading was obtained, or until sufficient data points were measured to allow extrapolation to a calculated maximum change.

The mean value of plateau current between 600 ms to 900 ms after application of glutamate or test compound plus glutamate was calculated and used as the parameter to measure the drug effect. The plateau responses in the presence of varying concentrations of test compound were divided by the baseline response in order to calculate the percentage increase. Compounds are deemed active in this test if, at a test concentration of 3 μM or less, they produce a greater than 100% increase in the value of the steady-state current measured due to application of glutamate alone. The concentration at which the glutamate induced current is increased by 100% is commonly referred to as the EC2x value. Compounds of the examples disclosed above displayed EC2x values in the range of 0.003 to 10 μM.

Rat Hippocampal Slice Assay

In another test, excitatory responses (field EPSPs) were measured in hippocampal slices, which were maintained in a recording chamber continuously perfused with artificial cerebrospinal fluid (ACSF). During a 15-30 minute interval, the perfusion medium was switched to one containing various concentrations of the test compounds. Responses collected immediately before and at the end of drug perfusion were superimposed in order to calculate the percent increase in EPSP amplitude.

The field EPSP (excitatory post-synaptic potential) recorded in field CA1 after stimulation of CA3 axons is known to be mediated by AMPA receptors, which are present in the synapses (Kessler et al., Brain Res. 560: 337-341 (1991)). Drugs that selectively block the receptor selectively block the field EPSP (Muller et al., Science, supra). Aniracetam, which has been shown to increase the mean open time of the AMPA receptor channel, increases the amplitude of the synaptic current and prolongs its duration (Tang et al., Science, supra).

These effects are mirrored in the field EPSP (see, for example, Staubli et al., Psychobiology, supra; Xiao et al., Hippocampus, supra; Staubli et al., Hippocampus 2: 4958 (1992)). Similar results have been reported for the previously disclosed stable benzamide analogs of aniracetam (Lynch and Rogers, PCT Pubn. No. WO 94/02475).

To obtain data for the activity of invention compounds on synaptic responses, a bipolar nichrome stimulating electrode was positioned in the dendritic layer (stratum radiatum) of the hippocampal subfield CA1 close to the border of subfield CA3. Current pulses (0.1 msec) through the stimulating electrode activate a population of the Schaffer-commissural (SC) fibers, which arise from neurons in the subdivision CA3 and terminate in synapses on the dendrites of CA1 neurons. Activation of these synapses causes them to release the transmitter glutamate. Glutamate binds to post-synaptic AMPA receptors, which then transiently open an associated ion channel and permit a sodium current to enter the postsynaptic cell. This current results in a voltage in the extracellular space (the field EPSP), which is recorded by a high impedance recording electrode positioned in the middle of the stratum radiatum of CA1.

The intensity of the stimulation current was adjusted to produce half-maximal EPSPs (typically about 1.5-2.0 mV). Paired stimulation pulses were given every 40 sec with an interpulse interval of 200 msec, as described further in Example 30.

Hippocampal slices were maintained in a recording chamber continuously perfused with artificial cerebrospinal fluid (ACSF). During 15-30 minute intervals, the perfusion medium was switched to one containing various concentrations of the test compounds. Responses collected immediately before and at the end of drug perfusion were superimposed in order to calculate the percent increase in EPSP amplitude.

Studies that compared the effects of AMPA modulators on monosynaptic (as reported here) and polysynaptic responses demonstrated that a 10% increase in the amplitude of the monosynaptic field EPSP was amplified to an increase of 300% on a trisynaptic response (Servio et al., Neuroscience 74: 1025-1035 (1996)). Furthermore, the concentration of the modulator that evoked these responses was shown to exist in plasma from behaviorally relevant doses (Granger et al., Synapse, supra). Thus, the concentration of compound sufficient to produce a 10% increase in amplitude of the monosynaptic field EPSP is likely to represent a behaviorally relevant plasma concentration.

II. In Vivo Physiological Testing

The physiological effects of invention compounds were tested in vivo in anesthetized animals according to the following procedures.

Animals are maintained under anesthesia by phenobarbital administered using a Hamilton syringe pump. Stimulating and recording electrodes are inserted into the perforant path and dentate gyrus of the hippocampus, respectively. Once electrodes are implanted, a stable baseline of evoked responses are elicited using single monophasic pulses (100 μs pulse duration) delivered at 3/min to the stimulating electrode. Field EPSPs are monitored until a stable baseline is achieved (about 20-30 min), after which a solution of test compound in HPCD is injected intraperitoneally and evoked field potentials are recorded. Evoked potentials are recorded for approximately 2 h following drug administration or until the amplitude of the field EPSP returns to baseline. In the latter instance, it is common that an iv administration is also carried out with an appropriate dose of the same test compound.

The activity of selected compounds of the invention in the patch clamp electrophysiology assay, the rat hippocampal slice assay and in the rat in vivo electrophysiology assay is summarized in Table 1.

TABLE 1

| Compound | In vitro Patch Clamp | ²Rat | |
|---|---|---|---|
| Example | Electrophysiology | Hippocampal | ^{3,4}In vivo |
| Number | ¹EC2x | slice assay | Electrophysiology |
| 1 | 0.28 μM | 69% @ 3 μM | 37%³ |
| 4 | 0.28 μM | 18% @ 3 μM | 46%³ |
| 18 | NT | 35% @ 10 μM | 34%³ |
| 22 | 0.24 μM | 24% @ 3 μM | 42%³ |
| 53 | 0.60 μM | 55% @ 10 μM | 33%³ |
| 80 | 0.016 μM | 65% @ 3 μM | 59%⁴ |

¹Concentration at which the glutamate induced current is increased by 100% in the patch clamp assay
²% Increase in the amplitude of the field EPSP in the CA1 region of rat hippocampal slice
³% increase in the amplitude of the field EPSP in rat dentate gyrus @ 5 mpk i.p.
⁴% increase in the amplitude of the field EPSP in rat dentate gyrus @ 1 mpk i.p.
NT = Not tested

While the invention has been described with reference to specific methods and embodiments, it will be appreciated that various modifications may be made without departing from the invention.

Administration, Dosages, and Formulation

As noted above, the compounds and method of the invention increase AMPA receptor-mediated responses, and are useful for the treatment of hypoglutamatergic conditions. They are also useful for treatment of conditions such as impairment of memory or other cognitive functions, brought on by a deficiency in the number or strength of excitatory synapses, or in the number of AMPA receptors. They may also be used in the treatment of schizophrenia or schizophreniform behavior resulting from a cortical/striatal imbalance, and in facilitation of learning of behaviors dependent upon AMPA receptors.

In subjects treated with the present compounds, pharmaceutical compositions and methods memory or other cognitive functions may be impaired, or cortical/striatal imbalance may occur, leading to loss of memory, dementia, depression, attention disorders, sexual dysfunction, movement disorders, schizophrenia or schizophreniform behavior. Memory disorders and learning disorders, which are treatable according to the present invention, include those disorders that result from aging, trauma, stroke and neurodegenerative disorders. Examples of neurodegenerative disorders include, but are not limited to, those associated with drug-induced states, neurotoxic agents, Alzheimer's disease, and aging. These conditions are readily recognized and diagnosed by those of ordinary skill in the art and treated by administering to the patient an effective amount of one or more compounds according to the present invention.

Generally, dosages and routes of administration of the compound will be determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g. orally, transdermally, perineurally or parenterally, that is, by intravenous, subcutaneous, intraperitoneal, or intramuscular injection, among others, including buccal, rectal and transdermal administration. Subjects contemplated for treatment according to the method of the invention include humans, companion animals, laboratory animals, and the like.

Formulations containing the compounds according to the present invention may take the form of solid, semi-solid, lyophilized powder, or liquid dosage forms, such as, for example, tablets, capsules, powders, sustained-release formulations, solutions, suspensions, emulsions, suppositories, creams, ointments, lotions, aerosols, patches or the like, preferably in unit dosage forms suitable for simple administration of precise dosages.

Pharmaceutical compositions according to the present invention typically include a conventional pharmaceutical carrier or excipient and may additionally include other medicinal agents, carriers, adjuvants, additives and the like. Preferably, the composition will be about 0.5 to 75% by weight of a compound or compounds of the invention, with the remainder consisting essentially of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

Liquid compositions can be prepared by dissolving or dispersing the compounds (about 0.5% to about 20% by weight or more), and optional pharmaceutical adjuvants, in a carrier, such as, for example, aqueous saline, aqueous dextrose, glycerol, or ethanol, to form a solution or suspension. For use in oral liquid preparation, the composition may be prepared as a solution, suspension, emulsion, or syrup, being supplied either in liquid form or a dried form suitable for hydration in water or normal saline.

When the composition is employed in the form of solid preparations for oral administration, the preparations may be tablets, granules, powders, capsules or the like. In a tablet formulation, the composition is typically formulated with additives, e.g. an excipient such as a saccharide or cellulose preparation, a binder such as starch paste or methyl cellulose, a filler, a disintegrator, and other additives typically used in the manufacture of medical preparations.

An injectable composition for parenteral administration will typically contain the compound in a suitable i.v. solution, such as sterile physiological salt solution. The composition may also be formulated as a suspension in a lipid or phospholipid, in a liposomal suspension, or in an aqueous emulsion.

Methods for preparing such dosage forms are known or will be apparent to those skilled in the art; for example, see Remington's Pharmaceutical Sciences (17th Ed., Mack Pub. Co., 1985). The composition to be administered will contain a quantity of the selected compound in a pharmaceutically effective amount for effecting increased AMPA receptor currents in a subject.

The following examples illustrate but are not intended in any way to limit the invention. Unless otherwise stated, all temperatures are given in degrees Celsius. Unless otherwise stated, all NMR spectra are ¹H NMR spectra and were obtained in deuterochloroform, deuterated DMSO or deuterated water as solvent using tetramethylsilane as an internal standard. All names of Example compounds conform to IUPAC nomenclature as provided by the computer software ChemSketch by ACD Labs.

### Chemical Procedures

### Intermediates 1 and 2

### (2R)-1-(2H-Tetrazol-2-yl)propan-2-amine hydrochloride and (2R)-1-(1H-Tetrazol-1-yl) propan-2-amine hydrochloride

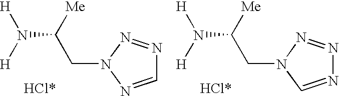

Tert-butyl [(2R)-1-hydroxypropan-2-yl]carbamate (12.3 g, 65.7 mmol), triphenylphosphine (26.3 g, 100 mmol) and tetrazole (0.45 molar in acetonitrile, 100 mmol) were dissolved in THF (100 ml). A solution of diisopropylazodicarboxylate (DIAD, 20.2 g, 100 mmol) in THF (50 ml) was added slowly and the mixture was stirred at 25° C. for 18 hr. The solvent was evaporated and the mixture purified using flash chromatography with ethyl acetate/hexane (30/70→60/40) as the mobile phase. The fractions containing the less polar 2-isomer were combined and the solvent was removed under vacuum (Rf: 0.65, white solid, ¹H NMR (300 MHz, CDCl₃) δ 8.52 (1H, s), 4.90-4.68 (3H, m), 4.33-4.20 (1H, m), 1.42 (9H, s) and 1.66 ppm (3H, d, J=6.9 Hz).

The fractions containing the more polar 1-substituted tetrazole were combined and the solvent was removed under vacuum (Rf: 0.20, white solid): ¹H NMR (300 MHz, CDCl₃) δ 8.65 (1H, s), 4.82-4.47 (3H, m), 4.14-4.01 (1H, m), 1.41 (9H, s) and 1.31 ppm (3H, d, J=6.6 Hz).

The fractions containing the 2-isomer were dissolved in chloroform (150 ml) and TFA (30 ml). The solvent was removed completely after 18 hours. Ethyl acetate (150 ml) was added and extracted with 2N HCl (100 and 50 ml). The water phase was evaporated, the remaining material re-dissolved in ethanol and again evaporated to give (2R)-1-(2H-tetrazol-2-yl)propan-2-amine hydrochloride (4.8 g) as a white solid: ¹H NMR (300 MHz, D₂O) δ 8.90 (1H, s), 5.46-5.32 and 5.17-5.09 (2H, m), 5.00-4.90 and 4.19-4.09 (1H, m), 1.44 and 1.31 ppm (3H, d+d, J=6.6 Hz).

The fractions containing the 1-substituted tetrazole were similarly treated with TFA and hydrochloric acid to give (2R)-1-(1H-tetrazol-1-yl)propan-2-amine hydrochloride as a white solid (3.5 g): ¹H NMR (300 MHz, D₂O) δ 9.33 and 9.22 (1H, s+s), 5.15-4.67 (2H, m), 4.12-4.00 (1H, m), 1.41 and 1.25 ppm (3H, d, J=6.6 Hz).

### Intermediates 3 and 4

### (2R)-1-(5-Methyl-2H-tetrazol-2-yl)propan-2-amine hydrochloride and (2R)-1-(5-Methyl-1H-tetrazol-1-yl)propan-2-amine hydrochloride

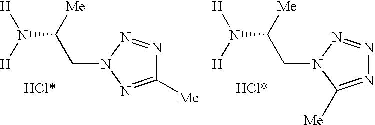

Intermediates 3 and 4 were prepared using the same procedures as for Intermediates 1 and 2 using 5-methyl tetrazole to give a ratio of 3:1 of 2-tetrazole:1-tetrazole. The 2-isomer was isolated as a white solid, ¹H NMR (300 MHz, D₂O) δ 5.28-5.20 and 5.06-4.92 (2H, m), 5.00-4.90 and 4.14-4.02 (1H, m), 2.58 (3H, s), 1.41 and 1.29 ppm (3H, d+d, J=6.6 Hz).

The 1-substituted tetrazole isomer was isolated in 23% yield.

### Intermediate 5

### Dimethyl-2-(formylamino) 1,4-dicarboxylate

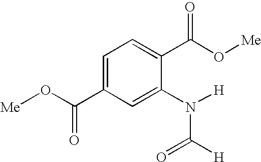

A mixture of acetic acid anhydride (100 ml) and formic acid (30 ml) was stirred for 10 minutes at RT and then added to a slurry of dimethyl-2-amino-terephthalate (25.0 g, 119.5 mmol) in chloroform (150 ml). The clear solution was stirred for 20 minutes at ambient temperature, after which the solvent was removed under vacuum to yield 28.4 g dimethyl 2-(formylamino) 1,4-dicarboxylate as an off white solid.

### Intermediate 6

### Dimethyl 2-(formylamino)-5-nitrobenzene-1,4-dicarboxylate

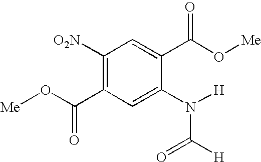

Powdered dimethyl 2-(formylamino)-1,4-dicarboxylate (28.4 g, 119.5 mmol) was added portion wise to cooled (5-10° C.) concentrated sulfuric acid (150 ml). After the material dissolved, the mixture was cooled to −10° C. and 90% HNO₃ (15 ml) was added slowly. The reaction was complete after 2.5 h (TLC: hexane/ethyl acetate/CHCl₃ 50/10/40). The reaction mixture was poured over crushed ice (500 g), extracted with dichloromethane (3×250 ml), washed with water (200 ml) and dried over sodium sulfate. The solution was filtered through silica gel and the solvent removed under vacuum to a volume of 200 ml, and MTBE (200 ml) was added and the solvent partly evaporated which caused the main isomer, dimethyl 2-(formylamino)-5-nitrobenzene-1,4-dicarboxylate, to crystallize (19.9 g; 59%). ¹H NMR (300 MHz, CDCl₃) δ 11.30 (1H, s), 9.05 (1H, s) 8.80 (1H, s), 8.60 (1H, s), 4.04 (3H, s), 3.99 ppm (3H, s).

### Intermediates 7 and 8

### Methyl 6-amino-3-cyclopropyl-4-oxo-3,4-dihydro-1,2,3-benzotriazine-7-carboxylate and Methyl 3-cyclopropyl-6-(formylamino)-4-oxo-3,4-dihydro-1,2,3-benzotriazine-7-carboxylate

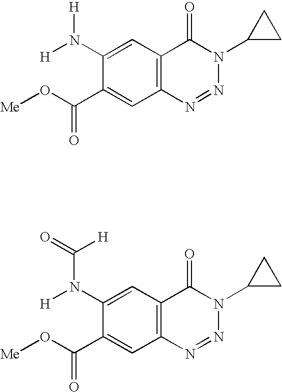

Dimethyl 2-(formylamino)-5-nitrobenzene-1,4-dicarboxylate (5.0 g, 17.7 mmol) was dissolved in THF (100 ml) and methanol (100 ml). Zn/Cu (30 g) and formic acid (6 ml) were added and stirred for 20 minutes. The mixture was filtered through 1.5 cm silica gel, washed with THF/MeOH (60 ml, 1:1) and the solvent evaporated, which yielded a yellow solid. This material was dissolved in THF (150 ml) and cooled to 0° C. A solution of sodium nitrite (2.5 g) in water (60 ml) was added, and conc. HCl (5 ml) was added (0° C.). After 7 minutes a mixture of cyclopropylamine (6 ml) and triethylamine (35 ml) were added in one portion and the mix was stirred for 1 hour without cooling. The solution was extracted with ethyl acetate (2×150 ml), dried over sodium sulfate and evaporated onto ˜10 g silica gel. Chromatography using ethyl acetate/hexane 40/60→ethyl acetate/hexane/methylene-chloride 60/20/20 yielded 4.5 g of a mixture of methyl 6-amino-3-cyclopropyl-4-oxo-3,4-dihydro-1,2,3-benzotriazine-7-carboxylate (INTERMEDIATE 7), ¹H NMR (300 MHz, CDCl₃) δ 8.68 (1H, s), 7.45 (1H, s), 6.45 (2H, s), 3.98 (3H, s), 3.98-3.83 (1H, m) and 1.30-1.12 ppm (4H, m), and methyl 3-cyclopropyl-6-(formylamino)-4-oxo-3,4-dihydro-1,2,3-benzotriazine-7-carboxylate (INTERMEDIATE 8), ¹H NMR (300 MHz, CDCl₃) δ 11.19 (1H, s), 9.58 (1H, s), 8.85 (1H, s), 8.63 (1H, s), 4.06 (1H, s), 4.06-3.90 (1H, m) and 1.38-1.17 ppm (4H, m).

### Intermediates 9 and 10

### Methyl 6-amino-3-methyl-4-oxo-3,4-dihydro-1,2,3-benzotriazine-7-carboxylate and Methyl 6-(formylamino)-3-methyl-4-oxo-3,4-dihydro-1,2,3-benzotriazine-7-carboxylate

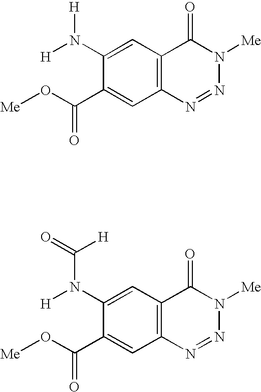

The title compounds were prepared according to the procedure used for intermediates 7 and 8 and using methylamine instead of cyclopropylamine. Intermediate 9: ¹H NMR (300 MHz, CDCl₃) δ 8.68 (1H, s), 7.45 (1H, s), 6.50 (2H, s) and 4.00 ppm (6H, s). Intermediate 10: ¹H NMR (300 MHz, CDCl₃) δ 11.2 (1H, s), 9.58 (1H, s), 8.88 (1H, s), 8.65 (1H, s) and 4.06 ppm (6H, s).

### Intermediate 11

### Methyl 3-ethyl-6-(formylamino)-4-oxo-3,4-dihydro-1,2,3-benzotriazine-7-carboxylate

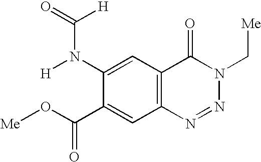

The title compound was prepared according to the procedure used for intermediates 7 and 8 and using ethylamine instead of cyclopropylamine. ¹H NMR (300 MHz, CDCl₃) δ 11.2 (1H, s), 9.56 (1H, s), 8.87 (1H, s), 8.63 (1H, s), 4.52 (2H, q, J=7.5 Hz), 4.06 (3H, s) and 1.52 ppm (3H, t, J=7.5 Hz).

### Intermediate 12

### Methyl 3-(2-fluoroethyl)-6-(formylamino)-4-oxo-3,4-dihydro-1,2,3-benzotriazine-7-carboxylate

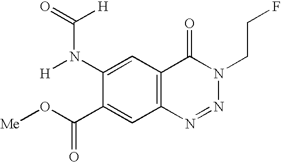

Prepared from 2-fluoro ethylamine using the procedure for intermediates 7 and 8, ¹H NMR (300 MHz, CDCl₃) δ 11.2 (1H, s), 9.60 (1H, s), 8.88 (1H, s), 8.65 (1H, s), 5.00-4.72 (4H, m) and 4.07 ppm (3H, s).

### Intermediate 13

### Methyl 6-(formylamino)-4-oxo-3-propan-2-yl-3,4-dihydro-1,2,3-benzotriazine-7-carboxylate

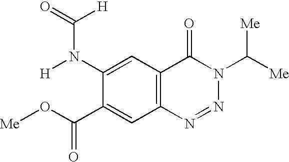

Prepared using previously described procedures: ¹H NMR (300 MHz, CDCl₃) δ 11.2 (1H, s), 9.55 (1H, s), 8.85 (1H, s), 8.63 (1H, s), 5.50-5.35 (1H, m), 4.06 (3H, s) and 1.58 ppm (3H, d, J=6.9 Hz).

### Intermediate 14

### Methyl 3-cyclobutyl-6-(formylamino)-4-oxo-3,4-dihydro-1,2,3-benzotriazine-7-carboxylate

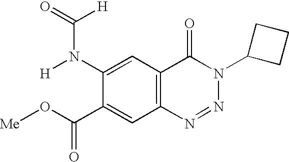

Prepared from cyclobutylamine using the procedure described for intermediates 7 and 8. ¹H NMR (300 MHz, CDCl₃) δ 11.2 (1H, s), 9.58 (1H, s), 8.90 (1H, s), 8.65 (1H, s), 5.62-5.48 (1H, m), 4.08 (3H, s), 2.87-2.70 (2H, m), 2.60-2.48 (2H, m) and 2.05-1.90 ppm (2H, m).

### Intermediate 15

### Methyl 3-(cyclopropylmethyl)-6-(formylamino)-4-oxo-3,4-dihydro-1,2,3-benzotriazine-7-carboxylate

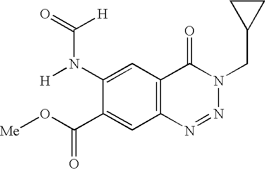

Prepared from cyclopropylmethylamine using the procedure described for intermediates 7 and 8, ¹H NMR (300 MHz, CDCl₃) δ 11.2 (1H, s), 9.58 (1H, s), 8.88 (1H, s), 8.64 (1H, s), 4.32 (2H, d, J=7.2 Hz), 4.08 (3H, s), 1.53-1.40 (1H, m) and 0.65-0.47 ppm (4H, m).

### Intermediate 16

### Methyl 6-(formylamino)-3(2-methylpropyl)-4-oxo-3,4-dihydro-1,2,3-benzotriazine-7-late

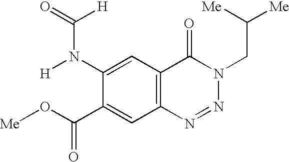

Prepared from isobutylamine using the procedure described for intermediates 7 and 8; ¹H NMR (300 MHz, CDCl₃) δ 11.2 (1H, s), 9.58 (1H, s), 8.85 (1H, s), 8.65 (1H, s), 4.29 (2H, d, J=6.9 Hz), 4.08 (3H, s), 2.45-2.30 (1H, m) and 1.03 ppm (3H, d, J=6.9 Hz).

### Intermediate 17

### Methyl 3-but-3-yn-1-yl-6-(formylamino)-4-oxo-3,4-dihydro-1,2,3-benzotriazine-7-carboxylate

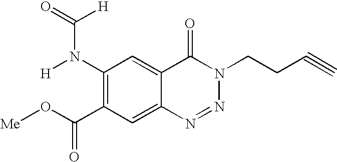

But-3-yn-1-ol (6.0 g, 85.7 mmol) was dissolved in anhydrous pyridine (50 ml) and cooled to 0° C., benzene sulfonyl chloride, (15 g, 85.7 mmol) was added within 30 minutes and stirred at room temperature for 3 hrs. The mixture was poured onto Ice/H₂SO₄ (400 ml), extracted with ethyl acetate (3×150 ml), dried over MgSO₄, and concentrated to yield 9.7 g of colorless oil. It was dissolved in DMF (50 ml), added NaN₃ (12.0 g) and stirred at 100° C. for 3 hours. The reaction mixture was cooled to room temperature after completion of the reaction. Freshly prepared Zn/Cu (15 g) was added into the reaction mixture, stirred fast and 4N HCl (˜6 ml) was added in portions. The warm solution was filtered, and the solvent evaporated. To the obtained white slush was added a cooled solution of NaOH (20 g) in water (100 ml). The water phase was extracted with ether (150 ml and 300 ml), dried over MgSO₄ and concentrated to give 1-amino-but-3-yne as a colorless oil. The amine was reacted with dimethyl 2-(formylamino)-5-nitrobenzene-1,4-dicarboxylate as described for intermediates 7 and 8 to give the title compound; ¹H NMR (300 MHz, CDCl₃) δ 11.20 (1H, s), 9.6 (1H, s), 8.88 (1H, s), 8.64 (1H, s), 4.63 (2H, t, J=6.9 Hz), 4.06 (3H, s), 2.85 (2H, dt, J=3.0 and 6.9 Hz) and 2.00 ppm (1H, t, J=3.0 Hz).

### Intermediate 18

### Dimethyl 2-[(ethoxycarbonyl)amino]-terephthalate

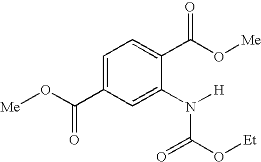

Dimethyl-2-amino-terephthalate (30.0 g, 143 mmol) was dissolved in dichloromethane (600 mL) and N-methylmorpholine (45 g, 445 mmol) was added. A solution of ethyl chloroformate (45 g, 415 mmol) in dichloromethane (100 ml) was slowly added. The mixture was stirred for 18 hours at ambient temperature, after which water (200 ml) and sulfuric acid (4 pH 2) were added, the organic phase was separated and the aqueous phase was washed with dichloromethane (200 mL). The combined organic phases were dried over sodium sulfate. The solvent was removed under vacuum and the material was crystallized from dichloromethane/methyl tert butylether (MTBE) to give the title compound as a white solid.

### Intermediate 19

### Dimethyl 2-[(ethoxycarbonyl)amino]-5-nitroterephthalate

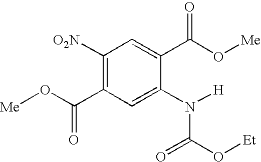

Powdered dimethyl-2-[(ethoxycarbonyl)amino]-terephthalate (3.73 g, 13 mmol) was added portionwise to cooled (−10° C.) 90% HNO₃ (20 ml). The reaction was complete after 10 minutes, as confirmed by TLC (hexane/ethyl acetate/CHCl₃ 60/20/20). The reaction mixture was poured over crushed ice (100 g), extracted with chloroform (2×100 ml), washed with saturated sodium bicarbonate (100 ml) and dried over sodium sulfate. The solvent was removed under vacuum and the resultant product was purified using flash chromatography with ethyl acetate/chloroform/hexane (20:20:60) as the mobile phase. Dimethyl 2-[(ethoxycarbonyl)amino]-5-nitroterephthalate was isolated as the faster eluting nitration product and was crystallized from methanol to give a white solid; ¹H NMR (300 MHz, CDCl₃) δ 10.81 (1H, s), 8.78 (1H, s) 8.77 (1H, s), 4.28 (2H, q, J=7.2 Hz), 4.00 (3H, s), 3.97 (3H, s) and 1.35 ppm (3H, t, J=7.2 Hz).

### Intermediates 20 and 21

### tert-Butyl [2-(2H-tetrazol-2-yl)ethyl]carbamate and tert-Butyl [2-(1H-tetrazol-1-yl)ethyl]carbamate

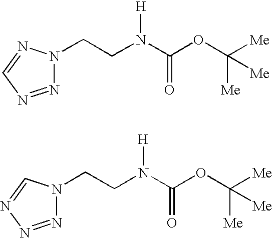

Hydroxyethyl phthalimide (3.0 g, 15.7 mmol), tetrazole (1.98 g, 28.3 mmol) and triphenyl phosphine (7.422 g, 28.3 mmol) were dissolved in THF (70 ml) and dichloromethane (70 ml). A solution of DIAD (5.73 g, 28.3 mmol) in dichloromethane (10 ml) was added slowly and the mixture stirred over night. NaHCO₃ solution (100 ml) was added and extracted with dichloromethane (2×100 ml). The solvent was evaporated and the residue dissolved in ethanol (150 ml). This mixture was heated with hydrazine (1.6 g) to 90° C. for 3 hours and the solvent evaporated. Ethylacetate (150 ml) and water (150 ml) were added and the pH adjusted to 2 using HCl. The water phase was re-extracted with ethyl acetate (150 ml) and KOH solution was added until the water phase reached pH 9. A solution of BOC anhydride (6 g) in ethyl acetate (150 ml) was added and the mixture was stirred over night. The water phase was extracted with ethyl acetate (150 ml), the solvent was evaporated and the mixture purified using chromatography (Hexane/ethyl acetate 60/40→40/60), which yielded tert-butyl [2-(2H-tetrazol-2-yl)ethyl]carbamate and tert-butyl [2-(1H-tetrazol-1-yl)ethyl]carbamate both as white solids.

### Intermediate 22

### Methyl-6-(formylamino)-3-(2-methoxyethyl)-4-oxo-3,4-dihydro-1,2,3-benzotriazine-7-carboxylate

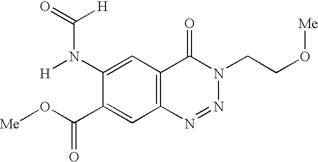

The title compound was prepared from 2-methoxy ethylamine following the procedure for intermediates 7 and 8; ¹H NMR (300 MHz, CDCl₃) δ 11.2 (1H, s), 9.58 (1H, s), 8.85 (1H, s), 8.63 (1H, s), 4.67 (2H, t, J=5.4 Hz), 4.07 (3H, s), 3.89 (2H, t, J=5.4 Hz), and 3.39 ppm (3H, s).

### Intermediates 23 and 24

### tert-Butyl [(2R)-1-(2H-tetrazol-2-yl)but-3-yn-2-yl]carbamate and tert-Butyl [(2R)-1-(1H-tetrazol-1-yl)but-3-yn-2-yl]carbamate

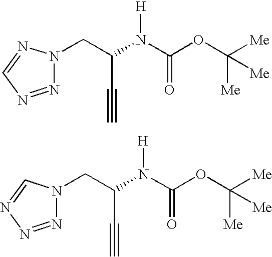

Glucosamine hydrochloride (60.0 g, 27.8 mmol) was suspended in methanol (400 ml) and a solution of NaOH (12 g) in water (200 ml) was added and stirred until clear. The mixture was cooled (ice bath) and a solution of BOC anhydride (62 g, 284 mmol) in THF (150 ml) was added slowly and the mixture was stirred over night. The white precipitate was filtered off and washed with MTBE. The filtrate was concentrated which yielded a second crop of product. The combined material was dried (oil pump) which yielded Boc-glucosamine.

30.2 g (108 mmol) of this material was suspended in ethanol (400 ml, 96%), sodium borohydride (4.16 g, 110 mmol) was added and stirred at RT for 2 hours. The solvent was evaporated and the resulting foam was dissolved in water (200 ml). Sulfuric acid was added until pH3, and the mixture was stirred for 5 minutes. The mixture was neutralized with NaHCO₃ and diluted with methanol (150 ml) and stirred at room temperature over night. The methanol was evaporated and the procedure repeated once more. The resulting clear solution was cooled to 5° C., and a solution of NaIO₄ (71 g, 332 mmol) in water (600 ml) was added slowly. The mixture was stirred at room temperature for 1 hour and extracted with dichloromethane (10×300 ml), dried over sodium sulfate and concentrated, which yielded tert-butyl [(2S)-1-hydroxy-3-oxopropan-2-yl]carbamate as a colorless foam. Dimethyl 2-oxopropylphosphonate (17.3 g, 104 mmol) and 4-(acetylamino)benzene sulfonylazide (25 g, 104 mmol) were dissolved in dry acetonitrile (250 ml), potassium carbonate (45 g) was added and the mixture was stirred for 2 hours under nitrogen. A solution of tert-butyl [(2S)-1-hydroxy-3-oxopropan-2-yl]carbamate (16.6 g, 87.7 mmol) in dry methanol (100 ml) was added and stirred at room temperature over night. Potassium carbonate (15 g) was added and the mixture was stirred for 24 hours. The solids were filtered off and the solvent evaporated, water (400 ml) were added and extracted with ethyl acetate (2×350 ml), dried over sodium sulfate and concentrated. Chromatography (Hexane/chloroform/THF 60/30/10) yielded tert-butyl [(2R)-1-hydroxybut-3-yn-2-yl]carbamate as a colorless oil.

Tert-butyl [(2R)-1-hydroxybut-3-yn-2-yl]carbamate (1.1 g, 5.8 mmol), tetrazole (740 mg, 10.5 mmol) and triphenylphosphine (2.75 g, 10.5 mmol) were dissolved in THF (50 ml) and dichloromethane (50 ml). A solution of DIAD (2.12 g, 10.5 mmol) in dichloromethane (5 ml) was added slowly and the mixture stirred over night. The solvent was evaporated and the mixture purified using chromatography (hexane/ethyl acetate 75/25→40/60), which yielded tert-butyl [(2R)-1-(2H-tetrazol-2-yl)but-3-yn-2-yl]carbamate (Intermediate 23): ¹H NMR (300 MHz, CDCl₃) δ 8.54 (1H, s), 5.25-5.12 (1H, m), 5.11-5.00 (1H, m), 5.00-4.84 (2H, m), 2.34 (1H, d, J=1.8 Hz) and 1.42 ppm (9H, s) and tert-butyl [(2R)-1-(1H-tetrazol-1-yl)but-3-yn-2-yl]carbamate (intermediate 24).

### Intermediates 25 and 26

### (2R)-1-(2H-1,2,3-triazol-2-yl)propan-2-amine hydrochloride and (2R)-1-(1H-1,2,3-triazol-1-yl)propan-2-amine hydrochloride

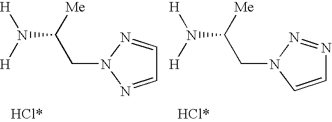

The title compounds were prepared as described for intermediates 1 and 2, using 1,2,3-triazole. (2R)-1-(2H-1,2,3-triazol-2-yl)propan-2-amine hydrochloride was as isolated as a white solid; ¹H NMR (300 MHz, D₂O) δ 7.84 (2H, s), 5.08-4.64 (2H, m), 4.05-3.90 (1H, m), 1.33 and 1.26 ppm (3H, d+d, J=6.6 Hz). (2R)-1-(1H-1,2,3-triazol-1-yl)propan-2-amine hydrochloride was as isolated as a white solid; ¹H NMR (300 MHz, D₂O) δ 8.29, 8.14 and 7.94 (2H, s+s+s), 5.05-4.95 and 4.05-3.95 (1H, m), 4.87-4.71 (2H, m), 1.38 and 1.31 ppm (3H, d+d, J=6.6 Hz).

### Intermediate 27

### (2R)-1-(1H-1,2,4-Triazol-1-yl)propan-2-amine hydrochloride

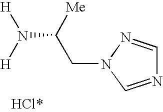

The title amine was prepared as described for intermediate 1 using 1,2,4-triazole; ¹H NMR (300 MHz, D₂O) δ 9.45 (1H, s), 8.67 (1H, s), 5.02-4.60 (2H, m), 4.15-4.00 (1H, m), 1.43 and 1.32 ppm (3H, d+d, J=6.6 Hz).

### Intermediate 28

### 1-(1H-Pyrazol-1-yl)propan-2-amine

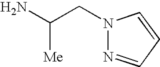

To a solution of pyrazole (3 g) in chloroacetone (10 ml) was added Cs₂CO₃ (325 mg, 1.0 mmol) and the mixture was heated to 60° C. for 4 h. The excess chloroacetone was evaporated and the residue was dissolved in chloroform (150 ml), washed with water (300 ml), dried over MgSO₄ and concentrated to give a dark mass. This material was subjected to chromatography on silica gel to give 2.2 g (17.7 mmol) of a yellow oil. The ketone was dissolved in ethanol (15 ml) hydroxylamine hydrochloride (1.22 g, 1.0 eq) and pyridine (1.0 ml) were added to the reaction mixture and heated at 50° C. for 1.5 h. The volatiles were evaporated and the residue was dissolved in chloroform (50 ml), washed with water (50 ml), dried over MgSO₄ and concentrated to give the dark oily mass (1.7 g). The oxime was dissolved in dry THF (50 ml) and added slowly into LiAlH₄ (2.32 g, 5.0 eq)/THF suspension. After addition was completed the reaction mixture was stirred at room temperature overnight. Hexane (50 ml) was added into the reaction mixture and cooled to 0° C. (ice bath). Conc. NaOH (10 g in 20 ml H₂O) was carefully added and the mixture stirred at room temperature for 1 hour. Filtration of the reaction mixture through a pad of celite, and concentration yielded the title compound as a yellow oil; ¹H NMR (300 MHz, CDCl₃) δ 7.52 (1H, sb), 7.41 (1H, d, J=2.1 Hz), 6.25 (1H, dd, J=2.1 Hz), 4.10 (1H, dd, J=4.5 and 13.5 Hz), 3.90 (1H, dd, J=8.4 and 13.5 Hz), 3.48-3.38 (1H, m) and 1.12 ppm (3H, d, J=6.3 Hz).

### Intermediate 29

### tert-Butyl [(2R)-1-thiocyanatopropan-2-yl]carbamate

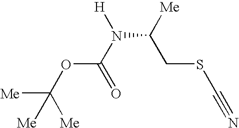

Tert-butyl [(2R)-1-hydroxypropan-2-yl]carbamate (5.0 g, 28.5 mmol) and triphenyl phosphine (13.8 g, 52.6 mmol) were dissolved in chloroform (100 ml) and THF (20 ml). N-Bromo succinimide (9.4 g, 52.8 mmol) was added portion wise and the mixture was stirred for 1 hour at RT. The material was evaporated onto silica gel and purified by chromatography with ethyl acetate/hexane (20/80) as the mobile phase. The fractions containing the product were combined and the solvent was removed under vacuum, which yielded a white solid; ¹H NMR (300 MHz, CDCl₃) δ 4.80-4.60 (1H, m), 4.02-3.90 (1H, m), 3.60-3.40 (2H, m), 1.45 (9H, s) and 1.25 ppm (3H, d, J=6.6 Hz). The preceding bromide (522 mg, 2.19 mmol) was dissolved in DMF (30 ml). Sodium thiocyanate (2.0 g) was added and the mixture was heated to 65° C. for 20 hours. The solvent was evaporated and the remaining material diluted with water (100 ml). The product was extracted with ethyl acetate/hexane (2:1, 2×100 ml) dried over sodium sulfate and concentrated to give an oil; ¹H NMR (300 MHz, CDCl₃) δ 4.75-4.65 (1H, m), 4.10-3.90 (1H, m), 3.30-3.05 (2H, m), 1.45 (9H, s) and 1.31 ppm (3H, d, J=6.9 Hz).

### Intermediates 30 and 31

### 2-[(2R)-1-(1-Methyl-1H-tetrazol-5-yl)propan-2-yl]-1H-isoindole-1,3(2H)-dione and 2-[(2R)-1-(2-Methyl-2H-tetrazol-5-yl)propan-2-yl]-1H-isoindole-1,3(2H)-dione

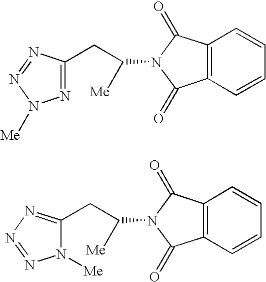

To a solution of 2-[(R)-2-hydroxy-1-methylethyl]-1H-isoindole-1,3(2H)-dione (9.0 g, 43.90 mmol) in CH₂Cl₂ (100 ml) was added methane sulfonic anhydride (11.45 g, 1.5 eq) in portions followed by TEA (15 ml) and stirred at room temperature for 1 h. The mixture was poured onto crushed ice (250 g), acidified with 2N HCl, extracted with chloroform (200 ml), dried over MgSO₄ and concentrated to give a yellow solid. The material was dissolved in DMSO (80 ml), NaCN (6.23 g, 3.0 eq) was added and heated to 100° C. for 3.5 h. The reaction mixture was cooled to room temperature and diluted with CH₂Cl₂/H₂O (200 ml, 1:1), extracted with CH₂Cl₂ (200 ml), washed with brine (200 ml), dried over MgSO₄ and concentrated to give a yellow oil. The product was purified using flash chromatography with hexane/ethyl acetate (4:1) to hexane/ethyl acetate (3:2) as the mobile phase. The solvent was evaporated to give a white solid (5.6 g).

The white solid was dissolved in toluene/DMF (150 ml, 2:1), sodium azide (6.8 g, 4.0 eq) and TEA.HCl (11.0 g, 3.0 eq) was added and heated to 115° C. overnight. The solvent was evaporated and diluted with H₂O (100 ml), acidified with 2N HCl, extracted with ethyl acetate (100 ml), 5% methanol in CHCl₃ (200 ml), dried over MgSO₄ and concentrated to give an orange oil (6.8 g).

The product was dissolved in DMF (100 ml), K₂CO₃ (9.12 g) and excess of MeI (16.5 ml, 10.0 eq) was added and stirred at room temperature over the weekend. The solvent was evaporated and the residue dissolved in ethyl acetate (150 ml) and H₂O (100 ml), the aqueous layer was extracted with CHCl₃ (200 ml), dried over MgSO₄ and concentrated to give a yellow oil. The product was purified using flash chromatography with hexane/ethyl acetate (4:1→3:2→1:1) as the mobile phase. The product fractions were concentrated to give 2-[(2R)-1-(1-methyl-1H-tetrazol-5-yl)propan-2-yl]-1H-isoindole-1,3(2H)-dione (intermediate 30); ¹H NMR (300 MHz, CDCl₃) δ 7.80-7.69 (4H, m), 5.00-4.84 (1H, m), 4.04 (3H, s), 3.76 (1H, dd, J=9.3 and 15.6 Hz), 3.32 (1H, dd, J=6.6 and 15.6 Hz) and 1.63 ppm (3H, d, J=6.9 Hz), and 2-[(2R)-1-(2-methyl-2H-tetrazol-5-yl)propan-2-yl]-1H-isoindole-1,3(2H)-dione (intermediate 31); ¹H NMR (300 MHz, CDCl₃) δ 7.82-7.68 (4H, m), 4.85-4.72 (1H, m), 4.21 (3H, s), 3.70 (1H, dd, J=9.3 and 15.0 Hz), 3.32 (1H, dd, J=9.0 and 15.0 Hz) and 1.61 ppm (3H, d, J=6.6 Hz).

### Intermediate 32

### tert-Butyl {(2R)-1-[(methylsulfonyl)amino]propan-2-yl}carbamate

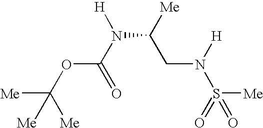

Tert-Butyl-[(2R)-1-hydroxypropan-2-yl]carbamate (3.5 g, 20 mmol) and triphenylphosphine (10.5 g, 40 mmol) were dissolved in 100 ml dry THF and cooled to −25° C. DIAD (8.1 g, 40 mmol) and diphenyl phosphoryl azide (11 g, 40 mmol) were added slowly. The mixture was slowly warmed up to RT and stirred for 1 hour. NaHCO₃ solution (100 ml) was added and the mixture extracted with ethyl acetate (2×100 ml), dried over sodium sulfate and concentrated to give an oil which was purified using chromatography (ethyl acetate/hexane 20/80) to yield the azide as an oil, which used as such in the next step; ¹H NMR (300 MHz, CDCl₃) δ 4.65-4.50 (1H, m), 3.90-3.80 (1H, m), 3.47-3.28 (2H, m), 1.46 (9H, s) and 1.19 ppm (3H, d, J=7.2 Hz).

The azide was dissolved in methanol (40 ml) and THF (40 ml), Zn/Cu (30 g) and formic acid (6 ml) were added and stirred for 15 minutes. The mixture was filtered and the filtrate evaporated. The residue was dissolved in chloroform (100 ml), THF (100 ml) and NEt₃ (10 ml). Methanesulfonyl chloride (2.4 g) was slowly added and stirred for 1 hour. Water (100 ml) and sulfuric acid were added (→pH 2), and extracted with dichloromethane (2×70 ml). The organic phase was washed with NaHCO₃ solution (100 ml), dried (sodium sulfate) and concentrated. This material was purified using chromatography (ethyl acetate/hexane/dichloromethane 50/40/10→dichloromethane/THF 60/40) to yield a white solid; ¹H NMR (300 MHz, CDCl₃) δ 5.10-4.98 (1H, m), 4.65-4.58 (1H, m), 3.92 (3H, s), 3.90-3.75 (1H, m), 3.35-3.05 (2H, m), 1.45 (9H, s) and 1.20 ppm (3H, d, J=6.9 Hz).

### Intermediate 33

### tert-Butyl {(2R)-1-[methyl(methylsulfonyl)amino]propan-2-yl}carbamate

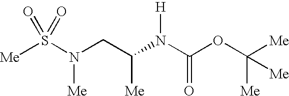

To a cooled (0° C., ice bath) solution of N-Boc-D-alaminol (4.0 g, 22.84 mmol) in CH₂Cl₂ (100 mL) was added triethyl amine (4.8 ml) and methane sulfonyl chloride (6.5 g, 2.5 eq) and the mixture was stirred for 1 hr. After completion of the reaction it was washed with H₂O (200 mL), brine (200 mL) dried over MgSO₄ and concentrated to give a white solid. The solid (2.1 g) was dissolved in CHCl₃ (30 ml), TEA (2 ml) was added and the reaction mixture was cooled to −78° C. (dry ice/acetone). Excess methylamine was added and stirred at room temperature and at 70° C. in a stainless steel pressure vessel overnight. The volatiles were evaporated and the residue dissolved in CH₂Cl₂ (50 ml), methane sulfonyl chloride (3.02 g, 2.5 eq) was added to the cooled reaction mixture (0° C.). Slowly TEA (2.0 ml) was added and stirred for 2 h. The solvents were evaporated and the product was purified using flash chromatography (ethyl acetate/hexane 20/80→30/70→50/50). The solvents were evaporated to give a white solid; ¹H NMR (300 MHz, CDCl₃) δ 4.68-4.58 (1H, m), 3.82-3.60 (1H, m), 3.22 (1H, dd, J=7.8 and 13.8 Hz), 3.01 (1H, dd, J=5.7 and 13.8 Hz), 2.92 (3H, s), 2.82 (3H, s), 1.44 (9H, s) and 1.18 (3H, d, J=6.9 Hz).

### Intermediate 34

### (1S)-1-(3-Fluorobenzyl)prop-2-ynylamine hydrochloride

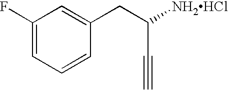

To a well stirred solution of Boc-3-fluoro-L-phenylalanine (2.00 g, 7.06 mmol in dichloromethane (10 ml), at ambient temperature under nitrogen, was added N,N-carbonyldiimidazole (1.32 g, 8.14 mmol) as a solid. Carbon dioxide evolution was observed and the mixture was stirred at ambient temperature for 3 hour. Solid N,O-dimethylhydroxylamine hydrochloride (0.878 g, 8.82 mmol) was added followed by the slow addition of triethylamine (1.23 ml, 8.82 mmol) via syringe. The cloudy slurry was diluted with dichloromethane (5 ml) to give a clear solution that was stirred at ambient temperature for 1 hour. The reaction mixture was poured into 10% citric acid (20 ml) and extracted with dichloromethane (4×20 ml). The organic phases were combined, dried over sodium sulfate and concentrated to give the crude product as a solid. This material was subjected to chromatography on silica gel (Merck Kieselgel 60, 230-400 mesh, 90 g, elution with 25% EtOAc/hexane) to give (S)-[2-(3-fluorophenyl)-1-(methoxymethyl-carbamoyl)-ethyl]-carbamic acid tert-butyl ester as a white crystalline solid: ¹H NMR (400 MHz, CDCl₃) δ 1.41 (9H, s), 2.87 (1H, dd, J=13.58, 7.36 Hz), 3.07 (1H, dd, J=13.6, 5.70 Hz), 3.20 (3H, s), 3.71 (3H, s), 4.94 (1H, m), 5.20 (1H, d, J=8.71 Hz), 6.93 (3H, m), 7.26 ppm (1H, m). MS (ESI+) for C₁₆H₂₃FN₂O₄ m/z 349.1 (M+Na)⁺.

To a cold (−45° C.; ACN—dry ice bath) well stirred slurry of (S)-[2-(3-fluorophenyl)-1-(methoxymethylcarbamoyl)ethyl]carbamic acid tert-butyl ester (11.46 g, 35.1 mmol) in dry diethyl ether (300 ml) was added LiAlH₄ (1.0 M in diethyl ether, 43.9 mmol) slowly via syringe. This mixture was stirred at −45° C. for 1 hour and slowly, carefully quenched with a solution of potassium bisulfate (8.37 g, 61.4 mmol) in water (80 ml). This slurry was allowed to warm to ambient temperature, diluted with ethyl acetate (200 ml) and filtered through a Celite plug. The solids were washed with ethyl acetate and the filtrates combined and separated. The organic phases were washed with 10% citric acid, saturated sodium bicarbonate, and brine, dried over sodium sulfate and concentrated. This provided (S)-[1-(3-fluorobenzyl)-2-oxo-ethyl]-carbamic acid tert-butyl ester as a white solid: ¹H NMR (400 MHz, CDCl₃) δ 1.45 (9H, s), 3.14 (2H, m), 4.43 (1H, m), 5.08 (1H, m), 6.96 (3H, m), 7.28 (1H, m), 9.65 ppm (1H, s). MS (ESI−) for C₁₄H₁₈FNO₃ m/z 266.2 (M-H)⁻

To a cold (−33° C.; FTS Flexicool) well-stirred mixture of carbon tetrabromide (23.3 g, 70.2 mmol), triphenylphosphine (36.8 g, 0.140 mol) and dichloromethane (500 ml) under nitrogen was dropwise added a solution of (S)-[1-(3-fluorobenzyl)-2-oxo-ethyl]-carbamic acid tert-butyl ester (9.38 g, 35.1 mmol) in dichloromethane (100 ml). Stirring was continued for 1 hour, the reaction was quenched with saturated sodium bicarbonate (100 ml) and extracted with methylene chloride (3×100 ml). The organic phases were combined, dried over sodium sulfate, and passed through a silica gel plug (˜100 g). This plug was washed with additional dichloromethane (200 ml), the filtrates were combined and concentrated. The residue was chromatographed on silica gel (Merck Kieselgel 60, 230-400 mesh, 350 g, elution with dichloromethane) and the mixed fractions re-chromatographed similarly. The pure fractions from both columns were combined to give 10.05 g of (S)-[3,3-dibromo-1-(3-fluorobenzyl)-allyl]-carbamic acid tert-butyl ester as a white solid. The product was re-crystallized from 1:1 hexane/ether to provide enantiomerically pure product: ¹H NMR (400 MHz, CDCl₃) δ 1.43 (9H, s), 2.91 (2H, m), 4.49 (1H, m), 4.57 (1H, m), 6.41 (1H, m), 6.95 (3H, m), 7.31 ppm (1H, m). MS (ESI+) for C₁₅H₁₈Br₂FNO₂ m/z 445.9 (M+Na)⁺

To a cold (−78° C.), well stirred solution of (S)-[3,3-dibromo-1-(3-fluorobenzyl)allyl]carbamic acid tert-butyl ester (4.25 g, 10.0 mmol) in dry tetrahydrofuran (50 mL) was added 1.35 M of n-butyllithium in hexane (23.8 ml) over a period of 15 min. After 2.5 hr at −78° C., the reaction was quenched with saturated ammonium chloride (30 ml) and diluted with diethyl ether (100 ml). This mixture was allowed to warm to ambient temperature and extracted with diethyl ether (4×100 ml). The organic phases were combined, dried over sodium sulfate and concentrated. The residue was chromatographed on silica gel (Merck Kieselgel 60, 230-400 mesh, 150 g, elution with 20% ethyl acetate/hexane) to give (S)-[1-(3-fluorobenzyl)-prop-2-ynyl]-carbamic acid tert-butyl ester which solidified under vacuum: ¹H NMR (400 MHz, CDCl₃) δ 1.45 (9H, s), 2.32 (1H, s), 2.97 (2H, m), 4.70 (2H, m), 7.01 (3H, m), 7.30 ppm (1H, m); ¹³C NMR. (CDCl₃) δ 162.67 (d, J=246 Hz), 154.52, 138.84 (d, J=7.3 Hz), 129.68 (d, J=8.1 Hz), 125.43 (d, J=2.9 Hz), 116.68 (d, J=21.2 Hz), 113.81 (d, J=20.5 Hz), 82.38, 80.15, 72.50, 43.70, 41.42, 28.29 ppm (3 C). MS (ESI+) for C₁₅H₁₈FNO₂ m/z 286.1 (M+Na)+

(S)-[1-(3-Fluorobenzyl)prop-2-ynyl]carbamic acid tert-butyl ester (1.1 g, 4.2 mmol) was dissolved in chloroform (25 ml) and trifluoroacetic acid (4 ml) was added. After one hour the solvent was evaporated by vacuum and methylene chloride (20 ml, saturated with HCl gas) was added, then removed by high vacuum to give the title product as an off white solid: ¹H NMR (300 MHz, CDCl₃/DMSO ˜9:1) δ 9.15 (3H, s), 7.32-7.25 (1H, m), 7.16-7.07 (2H, m), 7.00-6.93 (1H, m), 4.21-4.15 (1H, m), 3.48 (1H, dd, J=405 and 13.2 Hz), 3.14 (1H, dd, J=10.2 and 13.2 Hz), and 2.63 ppm (1H, d, J=2.1 Hz).

### Intermediates 35 and 36

### Methyl 6-amino-3-[(2R)-1-hydroxypropan-2-yl]-4-oxo-3,4-dihydro-1,2,3-benzotriazine-7-carboxylate and Methyl 6-(formylamino)-3-[(2R)-1-hydroxypropan-2-yl]-4-oxo-3,4-dihydro-1,2,3-benzotriazine-7-carboxylate

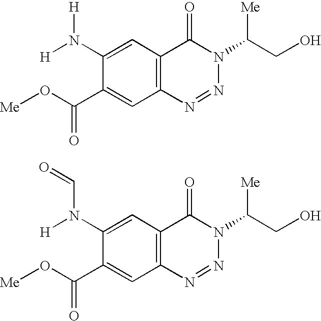

Intermediate 6 (3.0 g, 10.6 mmol) was dissolved in THF (60 ml) and methanol (60 ml). Zn/Cu (30 g) and formic acid (4 ml) were added and stirred for 15 minutes. The mixture was filtered through 1.5 cm silica gel, washed with THF/Methanol (1:1, 30 ml) and the solvent evaporated, which yielded a yellow solid. This material was dissolved in THF (100 ml) and cooled to 0° C. A solution of sodium nitrite (2.0 g) in water (60 ml) was added, and conc. HCl (4 ml) was added (0° C.). After 7 minutes a mixture of R-(−)-2-Amino-1-propanol (3 g) and triethylamine (10 ml) were added in one portion and the mix was stirred for 30 minutes without cooling. The solution was extracted with dichloromethane (2×100 ml), dried over sodium sulfate and evaporated. Crystallization from dichloromethane/methanol gave the title product as a beige solid.

### Intermediates 37 and 38

### Methyl 6-amino-4-oxo-3-[(2R)-1-(2H-1,2,3-triazol-2-yl)propan-2-yl]-3,4-dihydro-1,2,3-benzotriazine-7-carboxylate and Methyl 6-amino-4-oxo-3-[(2R)-1-(1H-1,2,3-triazol-1-yl)propan-2-yl]-3,4-dihydro-1,2,3-benzotriazine-7-carboxylate

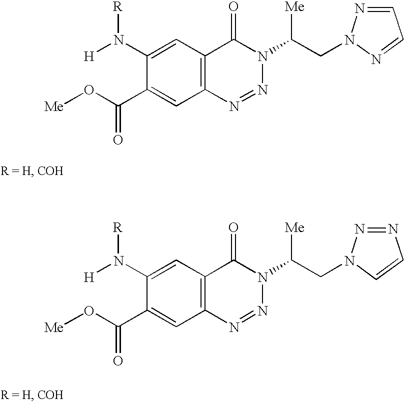

Intermediates 35 and 36 (1.5 g, 5.4 mmol), triphenylphosphine (2.1 g, 8.0 mmol) and 1,2,3-triazole (1 ml) were dissolved in THF (70 ml) and dichloromethane (70 ml). A solution of diisopropyl azodicarboxylate (DIAD, 1.62 g, 8.0 mmol) in THF (5 ml) was added slowly and the mixture was stirred at 25° C. for 1 hour. The solvent was evaporated and the mixture purified using flash chromatography (ethyl acetate/hexane 50/50→THF/dichloromethane 40/60). The fractions containing the less polar 2-substituted triazole isomer were combined and the solvent was removed under vacuum; ¹H NMR (300 MHz, CDCl₃) δ 8.70 (1H, s), 7.50 (2H, s), 7.41 (1H, s), 6.63-6.50 (2H, s), 5.83-5.71 (1H, m), 5.10 (1H, dd, J=8.4 and 13.8 Hz), 4.90 (1H, dd, J=5.1 and 13.8 Hz), 3.98 (3H, s) and 1.67 ppm (3H, d, J=6.9 Hz). The fractions containing the more polar 1-substituted triazole isomer were combined and the solvent was removed under vacuum.

### Intermediate 39

### Dimethyl 2-amino-5-(formylamino)benzene-1,4-dicarboxylate

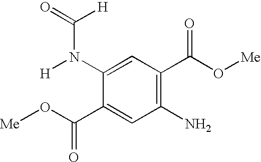

A solution of intermediate 6 in dichloromethane/methanol (2:1, 150 ml) was hydrogenated for 30 min at 50 psi over 10% Pd/C (1.0 g) in a Parr shaker. The solvent was removed under reduced pressure to give the title compound; ¹H NMR (300 MHz, DMSO+CDCl₃) δ 10.1 (1H, s), 8.89 (1H, s), 8.30 (1H, s), 7.44 (1H, s), 4.03 (2H, m), 3.83 (3H, s) and 3.80 ppm (3H, s).

### Intermediates 40 and 41

### Methyl 6-(formylamino)-4-oxo-3-[(2R)-1-(2H-tetrazol-2-yl)propan-2-yl]-3,4-dihydro-1,2,3-benzotriazine-7-carboxylate and Methyl 6-(formylamino)-4-oxo-3-[(2R)-1-(1H-tetrazol-1-yl)propan-2-yl]-3,4-dihydro-1,2,3-benzotriazine-7-carboxylate

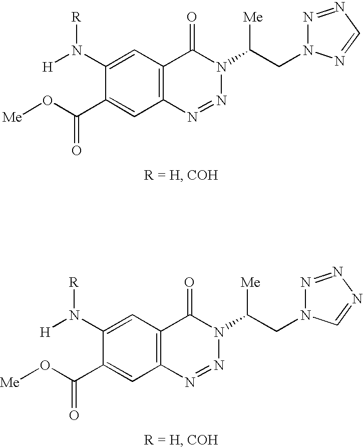

The title compounds were prepared from intermediates 35 and 36 and tetrazole using the procedure for intermediates 37 and 38. Intermediate 40 R═H had the following properties: ¹H NMR (300 MHz, CDCl₃) δ 8.69 (1H, s), 8.42 (1H, s), 7.35 (1H, s), 6.49 (2H, s), 5.83-5.70 (1H, m), 5.33 (1H, dd, J=8.4 and 13.8 Hz), 5.09 (1H, dd, J=5.1 and 13.8 Hz), 3.99 (3H, s) and 1.71 ppm (3H, d, J=7.2 Hz).

### Intermediate 42

### tert-butyl [(2R)-1-cyanopropan-2-yl]carbamate

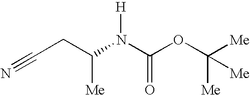

To a cooled (0° C.) solution of N-Boc-D-alaminol (2.0 g, 11.42 mmol) in CH₂Cl₂ (50 ml) was added triethylamine (2.5 ml) and methane sulfonyl chloride (3.25 g, 2.5 eq) and the mixture was stirred for 1 hour. After completion of the reaction it was poured onto sat. NaHCO₃ solution (100 ml) and extracted with CH₂Cl₂ (100 ml), dried over MgSO₄ and concentrated to give a white solid. The material was dissolved in DMSO (50 ml), NaCN (2.03 g, 3.0 eq) was added and heated to 100° C. for 90 min. The reaction mixture was cooled to room temperature, diluted with H₂O (100 ml), extracted with CHCl₃ (300 ml), washed with brine (100 ml), dried over MgSO₄ and concentrated to give yellow oil. The product was purified using flash chromatography with hexane/ethyl acetate (1:1) as the mobile phase. The solvent was evaporated to give a white solid; ¹H NMR (300 MHz, CDCl₃) δ 4.70-4056 (1H, m), 4.02-3.88 (1H, m), 2.75 (1H, dd, J=5.1 and 16.8 Hz), 2.53 (1H, dd, J=3.9 and 16.8 Hz), 1.45 (9H, s) and 1.33 ppm (3H, d, J=6.6 Hz).

### Intermediate 43

### 3-Cyclopropyl-8-[(2R)-1-hydroxypropan-2-yl]-3,8-dihydro[1,2,3]triazino[4,5-g]quinazoline-4,9-dione

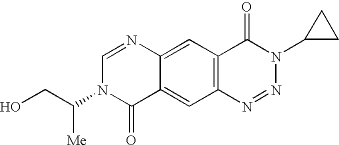

A mixture of intermediate 39 (62.4 mmol), (R)-2-amino-1-propanol (9 ml, 112 mmol), sodium cyanide (0.98 g, 20 mmol), and methanol (80 ml) was heated in a stream of argon with distillation of the solvent. After the entire methanol distilled off, heating was continued at 110° C. for 30 min. The crude product was purified by column chromatography (silica gel, chloroform/ethyl acetate/ethanol, 5:4:1). A suspension of the preceding alcohol (10.19 g, 36.7 mmol) in methanol (200 ml) was treated with solid 85% KOH (5.10 g, 77 mmol) and water (1.0 mL). The mixture was stirred and heated at reflux for 1 h. TLC indicated all the starting material was consumed. After cooling, the mixture was acidified by addition of conc. HCl (10 ml, 120 mmol), and the volatiles were removed under reduced pressure. DMF (150 ml) was added, and volatiles were thoroughly removed under reduced pressure. A solution of the obtained acid in anhydrous DMF (200 ml) was treated with EDCI (19.13 g, 100 mmol), 1-hydroxybenzotriazole hydrate (4.05 g, 30 mmol), DMAP (7.26 g, 60 mmol), cyclopropyl amine (8.4 ml, 120 mmol), and triethylamine (9.0 ml, 64.2 mmol). The reaction mixture was stirred under argon and heated at 40° C. for 6 h. The volatiles were removed under reduced pressure. A solution of the residue in chloroform (200 ml) was poured into brine (200 ml), acidified to pH 4 with 6N HCl and extracted with chloroform/ethanol (9:1, 10×200 ml). The combined extracts were dried over sodium sulfate, and the solvent was removed under reduced pressure to give the crude amide.

The crude amide was dissolved in DMF (100 ml) and treated with isobutyl nitrite (20 ml) and acetic acid (2 ml), and it was stirred at 22° C. for 16 h. The volatiles were removed under reduced pressure. Column chromatography of the residue (silica gel, chloroform/ethyl acetate/ethanol, 5:4:1) and re-crystallization of the product from ethyl acetate (50 ml) afforded 3-cyclopropyl-8-[(2R)-1-hydroxypropan-2-yl]-3,8-dihydro[1,2,3]triazino[4,5-g]quinazoline-4,9-dione; ¹H NMR (300 MHz, DMSO-d₆) δ 8.90 (1H, s), 8.48 (1H, s), 8.45 (1H, s), 4.95 (1H, m), 3.93 (1H, m), 3.86 (1H, dd, 6.6 and 11.7 Hz), 3.75 (1H, dd, J=4.5 and 11.7 Hz), 1.51 (3H, d, J=7.5 Hz), 1.28 (2H, m), and 1.21 (2H, m).

### Example 1

### 3-Cyclopropyl-8-[(1R)-1-methyl-2-(2H-tetrazol-2-yl)ethyl]-3,8-dihydro[1,2,3]triazino[4,5-g][1,2,3]-benzotriazine-4,9-dione

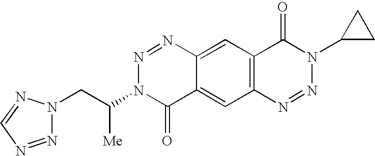

A mixture of intermediates 7 and 8 (2.70 g, 9.8 mmol) was dissolved in THF (100 ml) and methanol (75 ml). A solution of potassium hydroxide (6.0 g) in water (60 ml) was added, and the mixture was stirred (20° C.) for 45 minutes. Hydrochloric acid was added to the mixture (→pH 2) and the solvent evaporated. The mixture was dissolved in DMF (100 ml) and the solvent evaporated to remove water. (2R)-1-(2H-Tetrazol-2-yl)propan-2-amine hydrochloride (2.0 g, 10 mmol) and DMF (100 ml) were added and the solvent evaporated. DMF (80 ml), dimethylaminopiperidine (DMAP) (1.22 g, 10 mmol), hydroxybenzotriazole (HOBT) (1.35 g, 10 mmol), NEt₃ (2 ml) and N-(3-dimethylaminopropyl)-N′-ethylcarbodiimide (EDCI) (5.5 g, 28.5 mmol) were added and the mixture was stirred under nitrogen at 45° C. for 4 hours. The solvent was evaporated, water (100 ml) was added, the pH was adjusted to 2 using 2N H₂SO₄ and the mixture was extracted with ethyl acetate (3×200 ml). The organic phase was washed with saturated sodium bicarbonate solution (100 ml), dried over sodium sulfate and concentrated. The material was dissolved in DMF (100 ml), acetic acid (3 ml) and isoamyl nitrite (6 ml) were added. After 18 hours the solvent was evaporated and the product was purified using flash chromatography with ethyl acetate/hexane/dichloromethane (40/40/20) as the mobile phase. The product was crystallized from dichloromethane/MTBE/hexane to give a white solid (855 mg): mp=190-192° C.; ¹H NMR (300 MHz, CDCl₃) δ 9.14 (1H, s), 9.02 (1H, s), 8.40 (1H, s), 5.90-5.77 (1H, m), 5.36 (1H, dd, J=9 and 13.8 Hz), 5.15 (1H, dd, J=4.2 and 13.8 Hz), 4.10-4.00 (1H, m), 1.81 (3H, d, J=6.9 Hz) and 1.45-1.22 ppm (4H, m).

### Example 2

### 3-Cyclopropyl-8-[(2R)-1-(5-methyl-2H-tetrazol-2-yl)propan-2-yl]-3,8-dihydrobenzo[1,2-d:4,5-d′]bis[1,2,3]triazine-4,9-dione

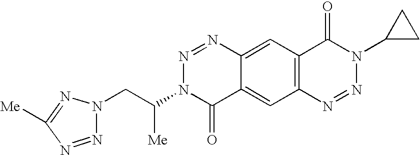

The title compound was prepared using the procedure of example 1 using intermediate 3. The resulting white solid had the following properties: mp=157-160° C.; ¹H NMR (300 MHz, CDCl₃) δ 9.15 (1H, s), 9.05 (1H, s), 5.90-5.75 (1H, m), 5.26 (1H, dd, J=8.4 and 14.4 Hz), 5.05 (1H, dd, J=4.8 and 14.4 Hz), 4.12-4.02 (1H, m), 2.44 (3H, s), 1.79 (3H, d, J=6.9 Hz) and 1.48-1.24 ppm (4H, m).

### Example 3

### 3-Cyclopropyl-8-[(2R)-1-(5-methyl-1H-tetrazol-1-yl)propan-2-yl]-3,8-dihydrobenzo[1,2-d:4,5-d′]bis[1,2,3]triazine-4,9-dione

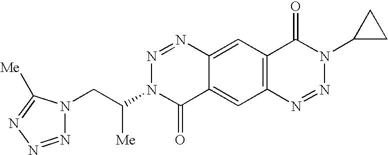

The title compound was prepared using the procedure of example 1 using intermediate 4. The resulting white solid had the following properties: mp=208-211° C.; ¹H NMR (300 MHz, CDCl₃) δ 9.14 (1H, s), 9.02 (1H, s), 5.90-5.79 (1H, m), 5.03 (1H, dd, J=9.6 and 14.4 Hz), 4.69 (1H, dd, J=5.1 and 14.4 Hz), 4.11-4.03 (1H, m), 2.62 (3H, s), 1.80 (3H, d, J=6.9 Hz) and 1.45-1.25 ppm (4H, m).

### Example 4

### 3-Methyl-8-[(2R)-1-(2H-tetrazol-2-yl)propan-2-yl]-3,8-dihydrobenzo[1,2-d:4,5-d′]bis[1,2,3]triazine-4,9-dione

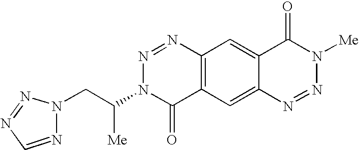

The title compound was prepared from intermediate 1 and intermediates 9 and 10 following the procedure used for example 1. The resulting white solid had the following properties: mp=228-230° C.; ¹H NMR (300 MHz, CDCl₃) δ 9.15 (1H, s), 9.06 (1H, s), 8.42 (1H, s), 5.90-5.80 (1H, m), 5.38 (1H, dd, J=8.7 and 13.8 Hz), 5.17 (1H, dd, J=4.2 and 13.8 Hz), 4.14 (3H, s) and 1.81 ppm (3H, d, J=6.9 Hz).

### Example 5

### 3-Methyl-8-[(2R)-1-(1H-tetrazol-1-yl)propan-2-yl]-3,8-dihydrobenzo[1,2-d:4,5-d′]bis[1,2,3]triazine-4,9-dione

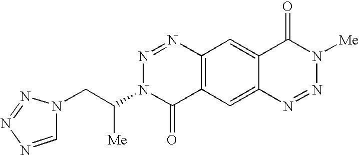

The title compound was prepared from intermediate 2 using the procedure described for example 1. The resulting white solid had the following properties: mp=228-230° C.; ¹H NMR (300 MHz, CDCl₃+CD₃OD) δ 9.14 (1H, s), 9.04 (1H, s), 8.94 (1H, s), 5.88-5.74 (1H, m), 5.22 (1H, dd, J=9.3 and 14.1 Hz), 5.00 (1H, dd, J=4.2 and 14.1 Hz), 4.14 (3H, s) and 1.78 ppm (3H, d, J=6.9 Hz).

### Example 6

### 3-Ethyl-8-[(2R)-1-(2H-tetrazol-2-yl)propan-2-yl]-3,8-dihydrobenzo[1,2-d:4,5-d′]bis[1,2,3]triazine-4,9-dione

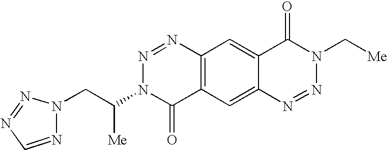

The title compound was prepared from intermediate 11 using the procedure of example 1. The compound was isolated as a white solid, mp=174-176° C.; ¹H NMR (300 MHz, CDCl₃) δ 9.13 (1H, s), 9.04 (1H, s), 8.40 (1H, s), 5.90-5.75 (1H, m), 5.36 (1H, dd, J=9.0 and 14.1 Hz), 5.15 (1H, dd, J=4.8 and 14.1 Hz), 4.59 (2H, q, J=7.2 Hz), 1.81 (3H, d, J=6.9 Hz) and 1.57 ppm (3H, t, J=7.2 Hz).

### Example 7

### 3-(2-Fluoroethyl)-8-[(2R)-1-(2H-tetrazol-2-yl)propan-2-yl]-3,8-dihydrobenzo[1,2-d:4,5-d′]bis[1,2,3]triazine-4,9-dione

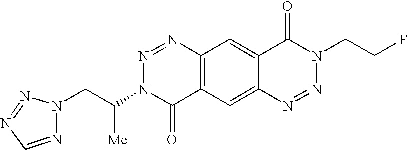

Title compound prepared from intermediate 12 using the procedure of example 1 and isolated as a white solid, mp=196-198° C.; ¹H NMR (300 MHz, CDCl₃) δ 9.15 (1H, s), 9.08 (1H, s), 8.41 (1H, s), 5.90-5.78 (1H, m), 5.37 (1H, dd, J=9.3 and 14.1 Hz), 5.17 (1H, dd, J=4.8 and 14.1 Hz), 5.04-4.78 (4H, m) and 1.82 ppm (3H, d, J=6.9 Hz).

### Example 8

### 3-Propan-2-yl-8-[(2R)-1-(2H-tetrazol-2-yl)propan-2-yl]-3,8-dihydrobenzo[1,2-d:4,5-d]′bis[1,2,3]triazine-4,9-dione

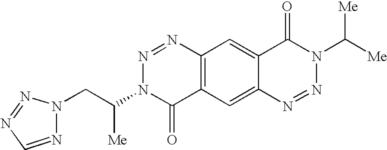

The title compound was prepared from intermediate 13 following the procedure used for example 1 and was isolated as a white solid, mp=134-136° C.; ¹H NMR (300 MHz, CDCl₃) δ 9.14 (1H, s), 9.04 (1H, s), 8.41 (1H, s), 5.90-5.78 (1H, m), 5.46 (1H, m), 5.37 (1H, dd, J=9.0 and 13.8 Hz), 5.16 (1H, dd, J=4.5 and 13.8 Hz), 1.82 (3H, d, J=6.9 Hz) and 1.64 ppm (6H, d, J=6.9 Hz).

### Example 9

### 3-Cyclobutyl-8-[(2R)-1-(2H-tetrazol-2-yl)propan-2-yl]-3,8-dihydrobenzo[1,2-d:4,5-d′]bis[1,2,3]triazine-4,9-dione

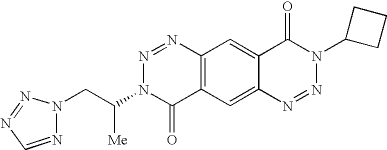

The title compound was prepared from intermediate 14 following the procedure used for example 1 and was isolated as a white solid, mp=152-154° C.; ¹H NMR (300 MHz, CDCl₃) δ 9.12 (1H, s), 9.05 (1H, s), 8.41 (1H, s), 5.90-5.78 (1H, m), 5.62-5.50 (1H, m), 5.37 (1H, dd, J=8.7 and 14.1 Hz), 5.16 (1H, dd, J=4.5 and 14.1 Hz), 2.86-2.72 (2H, m), 2.62-2.50 (2H, m), 2.10-1.94 (2H, m) and 1.81 ppm (3H, d, J=6.9 Hz).

### Example 10

### 3-(Cyclopropylmethyl)-8-[(2R)-1-(2H-tetrazol-2-yl)propan-2-yl]-3,8-dihydrobenzo[1,2-d:4,5-d′]bis[1,2,3]triazine-4,9-dione

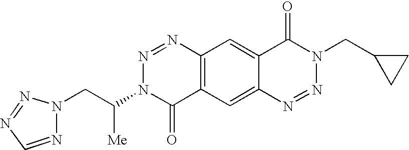

The title compound was prepared from intermediate 15 following the procedure used for example 1 and was isolated as a white solid, mp=134-136° C.; ¹H NMR (300 MHz, CDCl₃) δ 9.15 (1H, s), 9.05 (1H, s), 8.41 (1H, s), 5.90-5.78 (1H, m), 5.37 (1H, dd, J=8.7 and 13.8 Hz), 5.16 (1H, dd, J=4.5 and 13.8 Hz), 4.39 (2H, d, J=7.2 Hz), 1.82 (3H, d, J=7.2 Hz), 1.56-1.43 (1H, m) and 0.68-0.52 ppm (4H, m).

### Example 11

### 3-(2-Methylpropyl)-8-[(2R)-1-(2H-tetrazol-2-yl)propan-2-yl]-3,8-dihydrobenzo[1,2-d:4,5-d′]bis[1,2,3]triazine-4,9-dione

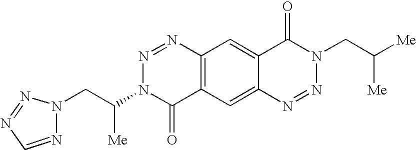

The title compound was prepared from intermediate 16 following the procedure used for example 1 and was isolated as a white solid, mp=132-134° C.; ¹H NMR (300 MHz, CDCl₃) δ 9.14 (1H, s), 9.04 (1H, s), 8.41 (1H, s), 5.90-5.78 (1H, m), 5.36 (1H, dd, J=9.0 and 14.1 Hz), 5.16 (1H, dd, J=4.8 and 14.1 Hz), 4.35 (2H, d, J=6.9 Hz), 2.48-2.33 (1H, m), 1.81 (3H, d, J=7.2 Hz) and 1.03 ppm (6H, d, J=6.9 Hz).

### Example 12

### 3-But-3-yn-1-yl-8-[(2R)-1-(2H-tetrazol-2-yl)propan-2-yl]-3,8-dihydrobenzo[1,2-d:4,5-d′]bis[1,2,3]triazine-4,9-dione

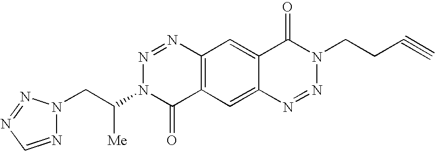

The title compound was prepared from intermediate 17 following the procedure used for example 1 and was isolated as a white solid, mp=185-188° C.; ¹H NMR (300 MHz, CDCl₃) 9.14 (1H, s), 9.06 (1H, s), 8.41 (1H, s), 5.90-5.78 (1H, m), 5.36 (1H, dd, J=9.3 and 14.1 Hz), 5.16 (1H, dd, J=4.8 and 14.1 Hz), 4.71 (2H, t, J=6.9 Hz), 2.90 (2H, dt, J=2.7 and 6.9 Hz), 2.02 (1H, t, J=2.7 Hz) and 1.81 ppm (3H, d, J=6.9 Hz).

### Example 13

### 3-But-3-yn-1-yl-8-[(2R)-1-(1H-tetrazol-1-yl)propan-2-yl]-3,8-dihydrobenzo[1,2-d:4,5-d′]bis[1,2,3]triazine-4,9-dione

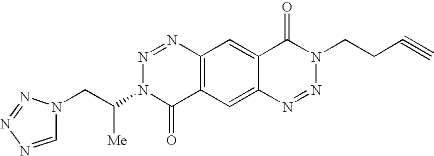

The title compound was prepared from intermediate 17 and intermediate 2 following the procedure used for example 1 and was isolated as a white solid, mp=201-204° C.; ¹H NMR (300 MHz, CDCl₃) δ 9.14 (1H, s), 9.05 (1H, s), 8.62 (1H, s), 5.90-5.78 (1H, m), 5.23 (1H, dd, J=9.3 and 14.1 Hz), 4.92 (1H, dd, J=4.8 and 14.1 Hz), 4.70 (2H, t, J=7.2 Hz), 2.90 (2H, dt, J=2.7 and 7.2 Hz), 2.01 (1H, t, J=2.7 Hz) and 1.78 ppm (3H, d, J=6.9 Hz).

### Example 14

### 3-Cyclopropyl-8-(1-Pyridin-3-ylpropan-2-yl)-3,8-dihydrobenzo[1,2-d:4,5-d′]bis[1,2,3]triazine-4,9-dione

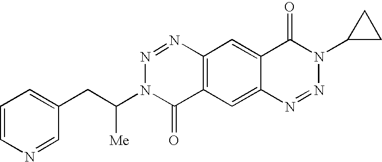

A mixture of nicotinaldehyde (6.2 g, 58 mmol), nitroethane (4.3 ml, 60 mmol) and isobutylamine (0.2 ml) was heated at 110° C. for 16 h. The crude product was purified by column chromatography (chloroform/ethyl acetate/ethanol, 50/45/5) to give pure nitro derivative (4.81 g, 50%). This material was added portionwise to a suspension of LiAlH₄ (4.55 g, 120 mmol) in ethyl ether (100 ml) and was stirred under argon at RT overnight and then heated at reflux for 5 h. After cooling in an ice/water bath, the reaction mixture was diluted with THF (100 ml), treated with sodium sulfate decahydrate (20 g) and stirred at for 1 h. The solid was filtered off, and the solvent was removed under reduced pressure to give the desired amine. The amine was reacted with a mixture of intermediates 7 and 8 using the procedure of example 1 to give the title compound as a white solid, mp=160-161° C.; ¹H NMR (300 MHz, CDCl₃) δ 9.11 (1H, s), 9.01 (1H, s), 8.41 (2H, m), 7.56 (1H, dt, J=7.9 and 1.8 Hz), 7.17 (1H, ddd, J=0.7, 4.8 and 7.7 Hz), 5.60 (1H, m), 4.04 (1H, m), 3.42 (1H, dd, J=8.8 and 13.9 Hz), 2.24 (1H, dd, J=6.6 and 14.3 Hz), 1.68 (3H, d, J=6.9 Hz), 1.37 (2H, m), and 1.27 (2H, m).

### Example 15

### 3-Cyclopropyl-8-[2-(2H-tetrazol-2-yl)ethyl]-3,8-dihydrobenzo[1,2-d:4,5-d′]bis[1,2,3]triazine-4,9-dione

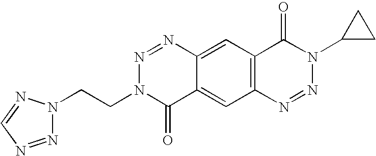

Prepared using previously described procedures from intermediate 20. The compound was isolated as a white solid; mp=250-252° C., ¹H NMR (300 MHz, CDCl₃) δ 9.12 (1H, s), 9.08 (1H, s), 8.48 (1H, s), 5.27 (2H, t, J=6.0 Hz), 5.07 (2H, t, J=6.0 Hz), 4.10-4.02 (1H, m) and 1.45-1.24 ppm (4H, m).

### Example 16

### 3-Cyclopropyl-8-[(2R)-1-(1H-tetrazol-1-yl)propan-2-yl]-3,8-dihydrobenzo[1,2-d:4,5-d′]bis[1,2,3]triazine-4,9-dione

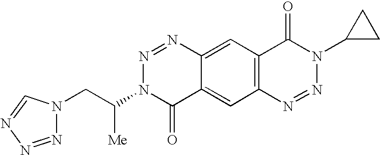

Dimethyl 2-[(ethoxycarbonyl)amino-5-nitroterephthalate (1.85 g, 5.67 mmol) was dissolved in THF (30 ml) and methanol (10 ml). A solution of KOH (1.68 g, 30 mmol) in water (30 ml) was added, and the mixture was stirred (70° C., N₂) for 18 hr. Hydrochloric acid was added to the cooled mixture (→pH2) and the solvent evaporated. The mixture was dissolved in DMF (100 ml) and the solvent evaporated to remove water. (2R)-1-(1H-tetrazol-1-yl)propan-2-amine hydrochloride (600 mg, 3 mmol) was dissolved in DMF (80 ml) and added to the crude 2-amino-5-nitroterephthalic acid. DMAP (733 mg, 6 mmol), HOBT (811 mg, 6 mmol), NEt₃ (3 ml) and EDCI (4.5 g, 23.5 mmol) were added and the mixture was stirred under nitrogen at 45° C. for 2.5 hours. Cyclopropylamine (1 ml) was added and the mixture was stirred at 45° C. for another 18 hours. The solvent was evaporated, water (100 ml) was added, the pH was adjusted to 2 using 2M H₂SO₄ and the mixture was extracted with AcOEt (2×100 ml). The organic phase was washed with saturated sodium bicarbonate solution (100 ml), dried over sodium sulfate and concentrated. The material was dissolved in THF (50 ml)/methanol (50 ml), Zn/Cu (12 g) and HCOOH (3 ml) were added and the mixture was stirred for 15 minutes. All solids were filtered off and the volatiles evaporated. The material was dissolved in DMF (30 ml) and isoamyl nitrite (5 ml) was added. After 4 hours the solvent was evaporated. The product was purified using flash chromatography with ethyl acetate/dichloromethane (65/35) as the mobile phase. The product crystallized when the solvent was evaporated. The resultant white solid had the following properties; mp=233-235° C.; ¹H NMR (300 MHz, CDCl₃) δ 9.14 (1H, s), 9.02 (1H, s), 8.60 (1H, s), 5.90-5.77 (1H, m), 5.25 (1H, dd, J=9.6 and 14.4 Hz), 4.91 (1H, dd, J=4.8 and 14.4 Hz), 4.13-4.00 (1H, m), 1.77 (3H, d, J=6.6 Hz) and 1.45-1.22 ppm (4H, m).

### Example 17

### 3-Cyclopropyl-8-[(2R)-1-(2H-tetrazol-2-yl)propan-2-yl]-3,8-dihydropyrimido[4,5-g]quinazoline-4,9-dione

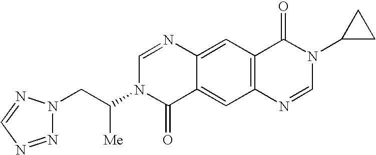

Dimethyl 2-[(ethoxycarbonyl)amino-5-nitroterephthalate (1.85 g, 5.67 mmol) was dissolved in THF (30 ml) and Methanol (10 ml). A solution of KOH (1.68 g, 30 mmol) in water (30 ml) was added, and the mixture was stirred (70° C., N₂) for 18 hr. Hydrochloric acid was added to the cooled mixture (→pH2) and the solvent evaporated. The mixture was dissolved in DMF (100 ml) and the solvent evaporated to remove water. (2R)-1-(1H-tetrazol-1-yl)propan-2-amine hydrochloride (600 mg, 3 mmol) was dissolved in DMF (80 ml) and added to the crude 2-amino-5-nitroterephthalic acid. DMAP (733 mg, 6 mmol), HOBT (811 mg, 6 mmol), NEt₃ (3 ml) and EDCI (4.5 g, 23.5 mmol) were added and the mixture was stirred under nitrogen at 45° C. for 2.5 hours. Cyclopropylamine (1 ml) was added and the mixture was stirred at 45° C. for another 18 hours. The solvent was evaporated, water (100 ml) was added, the pH was adjusted to 2 using 2M H₂SO₄ and the mixture was extracted with ethyl acetate (2×100 ml). The organic phase was washed with saturated sodium bicarbonate solution (100 ml), dried over sodium sulfate and concentrated. The material was dissolved in THF (50 ml)/Methanol (50 ml), Zn/Cu (12 g) and HCOOH (3 ml) were added and the mixture was stirred for 15 minutes. All solids were filtered off and the volatiles evaporated. The material was dissolved in DMF (50 ml) and toluene (250 ml), and toluene sulfonic acid (500 mg) was added. The mixture was heated and ˜10 ml toluene distilled off. While distilling, a solution of trimethyl orthoformate (5 ml) in toluene (5 ml) was added portion wise within 15 minutes. After cooling, ethyl acetate (200 ml) was added and the mixture was extracted with saturated sodium bicarbonate solution (100 ml), dried over sodium sulfate and concentrated. The product was purified using flash chromatography with chloroform/ethyl acetate/methanol (60/36/4) as the mobile phase. The solvent was evaporated and the product crystallized from dichloromethane/ethyl acetate. The resulting white solid had the following properties; mp=221-223° C.; ¹H NMR (300 MHz, CDCl₃) δ 8.62 (1H, s), 8.60 (1H, s), 8.46 (1H, s), 8.14 (1H, s), 7.72 (1H, s), 5.39 (1H, dd, J=7.8 and 13.5 Hz), 5.22-5.05 (1H, m), 5.06 (1H, dd, J=4.8 and 13.8 Hz), 3.31-3.21 (1H, m), 1.77 (3H, d, J=6.9 Hz), 1.30-1.15 (2H, m) and 1.05-0.93 ppm (4H, m).

### Example 18

### 3-(2-Methoxyethyl)-8-[(2R)-1-(2H-tetrazol-2-yl)propan-2-yl]-3,8-dihydrobenzo[1,2-d: 4,5-d′]bis[1,2,3]triazine-4,9-dione

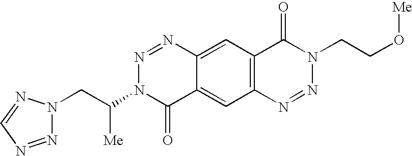

Prepared from intermediate 22 using the procedure of example 1. The resulting white solid had the following properties; mp=145-147° C.; ¹H NMR (300 MHz, CDCl₃) δ 9.15 (1H, s), 9.06 (1H, s), 8.41 (1H, s), 5.90-5.80 (1H, m), 5.37 (1H, dd, J=8.7 and 13.8 Hz), 5.17 (1H, dd, J=4.5 and 13.8 Hz), 4.75 (2H, t, J=5.7 Hz), 3.93 (2H, t, J=5.7 Hz), 3.40 (3H, s) and 1.82 ppm (3H, d, J=7.2 Hz).

### Example 19

### 3-(2-Methoxyethyl)-8-[(2R)-1-(2H-tetrazol-2-yl)propan-2-yl]-3,8-dihydro[1,2,3]triazino[4,5-g]quinazoline-4,9-dione

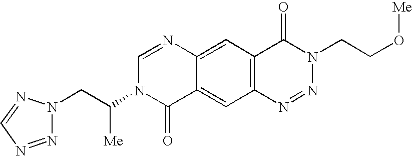

Intermediate 22 (460 mg, 1.50 mmol) was dissolved in THF (80 ml). A solution of potassium hydroxide (1.0 g) in water (20 ml) was added, and the mixture was stirred at RT for 90 minutes. Hydrochloric acid was added to the cooled mixture (→pH2) and the solvent evaporated. The mixture was dissolved in DMF (100 ml) and the solvent evaporated to remove the water. DMF (70 ml), intermediate 1 (310 mg, 1.55 mmol), DMAP (189 mg, 1.55 mmol), HOBT (209 mg, 1.55 mmol), NEt₃ (2 ml) and EDCI (2.0 g, 10.4 mmol) were added and the mixture was stirred under nitrogen at 55° C. for 2 hours. The solvent was evaporated, water (100 ml) was added, the pH was adjusted to 2 using 2M H₂SO₄ and the mixture was extracted with dichloromethane/THF (2:1, 2×150 ml). The organic phase was washed with saturated sodium bicarbonate solution (100 ml), dried over sodium sulfate and concentrated. The material was dissolved in dioxane (150 ml), then p-toluene sulfonic acid (600 mg) was added. The mixture was heated and ˜10 ml solvent distilled off. While distilling, a solution of trimethyl orthoformate (5 ml) in dioxane (5 ml) was added portion wise (1 ml/minute). After cooling, saturated sodium bicarbonate solution (100 ml) was added and the mixture was extracted with ethyl acetate (2×100 ml), dried over sodium sulfate and concentrated. The product was purified using flash chromatography with toluene/acetone (80/20) as the mobile phase. The resulting foam had the following properties: ¹H NMR (300 MHz, CDCl₃) δ 9.09 (1H, s), 8.63 (1H, s), 8.47 (1H, s), 7.81 (1H, s), 5.40 (1H, dd, J=7.5 and 13.8 Hz), 5.25-5.10 (1H, m), 5.08 (1H, dd, J=4.5 and 13.8 Hz), 4.70 (2H, t, J=5.7 Hz), 3.90 (2H, t, J=5.7 Hz), 3.39 (3H, s) and 1.79 ppm (3H, d, J=7.2 Hz).

### Example 20

### 3-(Pyridin-3-ylmethyl)-8-[(2R)-1-(2H-tetrazol-2-yl)propan-2-yl]-3,8-dihydrobenzo[1,2-d:4,5-d′]bis[1,2,3]triazine-4,9-dione

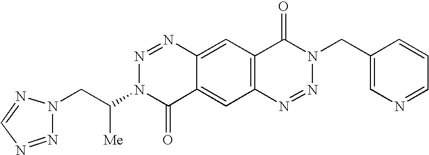

Prepared from 1-pyridin-3-ylmethanamine and intermediate 19 using the procedure of example 16. The resulting white solid had the following properties: mp: ˜200° C. degradation, ¹H NMR (300 MHz, CD₃OD/CDCl₃) δ 9.07 (1H, s), 8.89 (1H, s), 8.87 (1H, s), 8.67 (1H, d, J=5.4 Hz), 8.54 (1H, d, J=8.1 Hz), 8.26 (1H, s), 7.90 (1H, dd, J=5.4 and 8.1 Hz), 5.72 (2H, s), 5.70-5.58 (1H, m), 5.18 (1H, dd, J=8.7 and 14.1 Hz), 5.03 (1H, dd, J=4.5 and 14.4 Hz) and 1.63 ppm (3H, d, J=6.9 Hz).

### Example 21

### 3-Prop-2-yn-1-yl-8-[(2R)-1-(2H-tetrazol-2-yl)propan-2-yl]-3,8-dihydrobenzo[1,2-d:4,5-d′]bis[1,2,3]triazine-4,9-dione

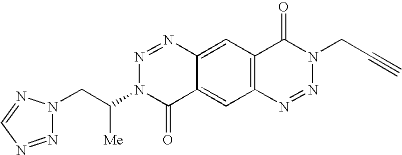

Prepared from propargylamine using previously described procedures and isolated as a white solid; mp=209-211° C., ¹H NMR (300 MHz, CDCl₃) δ 9.17 (1H, s), 9.09 (1H, s), 8.42 (1H, s), 5.90-5.80 (1H, m), 5.38 (1H, dd, J=8.7 and 14.1 Hz), 5.29 (2H, d, J=2.4 Hz), 5.17 (1H, dd, J=4.8 and 14.1 Hz), 2.45 (1H, t, J=2.4 Hz) and 1.83 ppm (3H, d, J=6.9 Hz).

### Example 22

### {4,9-Dioxo-8-[(2R)-1-(2H-tetrazol-2-yl)propan-2-yl]-8,9-dihydrobenzo[1,2-d:4,5-d′]bis[1,2,3]triazin-3(4H)-yl}acetonitrile

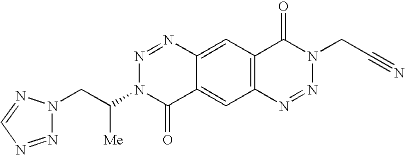

Prepared from intermediate 1 and aminoacetonitrile using the procedure of example 16. The title compound was isolated as a white solid; mp=209-212° C., ¹H NMR (300 MHz, CDCl₃) δ 9.17 (1H, s), 9.12 (1H, s), 8.42 (1H, s), 5.92-5.80 (1H, m), 5.38 (2H, s), 5.37 (1H, dd, J=9.0 and 13.8 Hz), 5.18 (1H, dd, J=4.5 and 13.8 Hz) and 1.84 ppm (3H, d, J=6.9 Hz).

### Example 23

### 3-Cyclopropyl-8-[(2R)-1-(2H-tetrazol-2-yl)but-3-yn-2-yl]-3,8-dihydrobenzo[1,2-d:4,5-d′]bis[1,2,3]triazine-4,9-dione

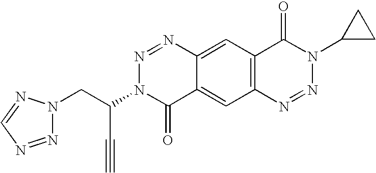

The title compound was prepared from intermediate 23 (de-protected with TFA) and intermediate 19 using the procedure of example 16. The resulting white solid had the following properties: mp=224-225° C., ¹H NMR (300 MHz, CDCl₃) δ 9.16 (1H, s), 9.03 (1H, s), 8.43 (1H, s), 6.50-6.43 (1H, m), 5.58 (1H, dd, J=9.0 and 13.8 Hz), 5.39 (1H, dd, J=4.8 and 13.8 Hz), 4.10-4.02 (1H, m), 2.69 (1H, d, J=2.4 Hz) and 1.45-1.22 ppm (4H, m).

### Example 24

### 3-Cyclopropyl-8-[(2R)-1-(1H-tetrazol-1-yl)but-3-yn-2-yl]-3,8-dihydrobenzo[1,2-d:4,5-d′]bis[1,2,3]triazine-4,9-dione

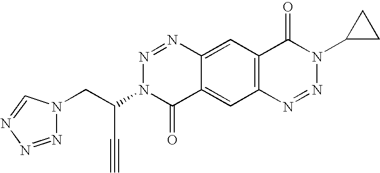

Prepared from intermediate 24 (de-protected with TFA) and intermediate 19 following the procedure of example 16. The resulting white solid had the following properties: mp>195° C. (degradation), ¹H NMR (300 MHz, CDCl₃) δ 9.17 (1H, s), 9.07 (1H, s), 8.75 (1H, s), 6.46-6.41 (1H, m), 5.31 (1H, dd, J=7.5 and 14.1 Hz), 5.20 (1H, dd, J=6.0 and 14.1 Hz), 4.11-4.03 (1H, m), 2.70 (1H, d, J=2.1 Hz) and 1.45-1.24 ppm (4H, m).

### Example 25

### 3-Cyclopropyl-8-[(2R)-1-(2H-1,2,3-triazol-2-yl)propan-2-yl]-3,8-dihydrobenzo[1,2-d:4,5-d′]bis[1,2,3]triazine-4,9-dione

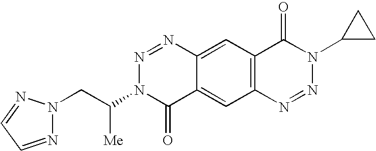

Prepared using previously described procedures from intermediate 25 and isolated as a white solid; mp=189-192° C., ¹H NMR (300 MHz, CDCl₃) δ 9.14 (1H, s), 9.04 (1H, s), 7.48 (2H, s), 5.87-5.75 (1H, m), 5.11 (1H, dd, J=9.0 and 14.1 Hz), 4.94 (1H, dd, J=4.8 and 14.1 Hz), 4.10-4.02 (1H, m), 1.77 (3H, d, J=6.9 Hz) and 1.43-1.24 ppm (4H, m).

### Example 26

### 3-Cyclopropyl-8-[(2R)-1-(1H-1,2,3-triazol-1-yl)propan-2-yl]-3,8-dihydrobenzo[1,2-d:4,5-d′]bis[1,2,3]triazine-4,9-dione

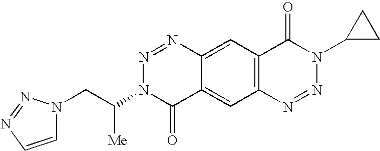

Prepared using previously described procedures from intermediate 26 and isolated as a white solid; mp: 200-203° C., ¹H NMR (300 MHz, CDCl₃) δ 9.14 (1H, s), 9.03 (1H, s), 7.62 (1H, s), 7.58 (1H, s), 5.88-5.76 (1H, m), 5.14 (1H, dd, J=9.0 and 14.4 Hz), 4.88 (1H, dd, J=5.1 and 14.4 Hz), 4.10-4.03 (1H, m), 1.77 (3H, d, J=6.9 Hz) and 1.44-1.22 ppm (4H, m).

### Example 27

### 3-Methyl-8-[(2R)-1-(2H-1,2,3-triazol-2-yl)propan-2-yl]-3,8-dihydrobenzo[1,2-d:4,5-d′]bis[1,2,3]triazine-4,9-dione

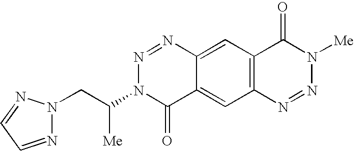

Prepared using previously described procedures from intermediate 25 and isolated as a white solid; mp=202-204° C., ¹H NMR (300 MHz, CDCl₃) δ 9.14 (1H, s), 9.06 (1H, s), 7.48 (2H, s), 5.87-5.75 (1H, m), 5.14 (1H, dd, J=8.7 and 13.8 Hz), 4.95 (1H, dd, J=4.5 and 13.8 Hz), 4.14 (3H, s) and 1.77 (3H, d, J=7.2 Hz).

### Example 28

### 3-Methyl-8-[(2R)-1-(1H-1,2,3-triazol-1-yl)propan-2-yl]-3,8-dihydrobenzo[1,2-d:4,5-d′]bis[1,2,3]triazine-4,9-dione

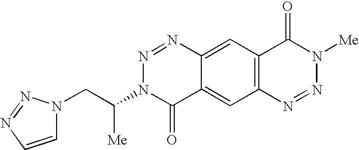

Prepared using previously described procedures from intermediate 26 and isolated as a white solid; mp=231-233° C., ¹H NMR (300 MHz, CDCl₃) δ 9.14 (1H, s), 9.05 (1H, s), 7.62 (1H, s), 7.58 (1H, s), 5.88-5.77 (1H, m), 5.14 (1H, dd, J=9.6 and 14.1 Hz), 4.87 (1H, dd, J=5.1 and 14.1 Hz), 4.14 (3H, s) and 1.77 (3H, d, J=6.6 Hz).

### Example 29

### 3-(2-Methoxyethyl)-8-[(2R)-1-(2H-1,2,3-triazol-2-yl)propan-2-yl]-3,8-dihydrobenzo[1,2-d:4,5-d′]bis[1,2,3]triazine-4,9-dione

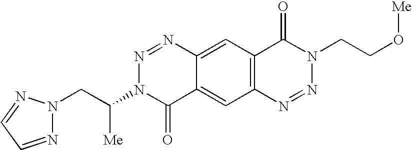

The title compound was prepared from intermediates 22 and 25 using the procedure of example 1; mp=145-146° C., ¹H NMR (300 MHz, CDCl₃) δ 9.13 (1H, s), 9.05 (1H, s), 7.47 (2H, s), 5.86-5.74 (1H, m), 5.10 (1H, dd, J=9.0 and 14.4 Hz), 4.94 (1H, dd, J=4.8 and 14.4 Hz), 4.73 (2H, t, J=5.1 Hz), 3.92 (2H, t, J=5.1 Hz), 3.39 (3H, s) and 1.76 (3H, d, J=7.2 Hz).

### Example 30

### 3-Cyclopropyl-8-[(2R)-1-(1H-1,2,4-triazol-1-yl)propan-2-yl]-3,8-dihydrobenzo[1,2-d:4,5-d′]bis[1,2,3]triazine-4,9-dione

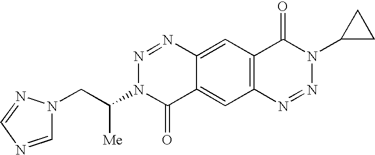

Prepared from intermediate 27 using previously described procedures; mp=195-199° C., ¹H NMR (300 MHz, CDCl₃) δ 9.14 (1H, s), 9.04 (1H, s), 8.02 (1H, s), 7.83 (1H, s), 5.90-5.80 (1H, m), 4.96 (1H, dd, J=9.3 and 13.8 Hz), 4.67 (1H, dd, J=5.1 and 13.8 Hz), 4.10-4.03 (1H, m), 1.73 (3H, d, J=6.6 Hz) and 1.45-1.25 ppm (4H, m).

### Example 31

### 3-Cyclopropyl-8-[1-(1H-pyrazol-1-yl)propan-2-yl]-3,8-dihydrobenzo[1,2-d:4,5-d′]bis[1,2,3]triazine-4,9-dione

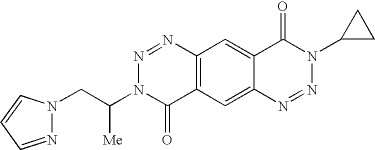

Prepared from intermediate 28 using previously described procedures; mp=178-180° C.; ¹H NMR (300 MHz, CDCl₃) δ 9.11 (1H, s), 9.02 (1H, s), 7.37 (1H, d, J=1.8 Hz), 7.30 (1H, d, J=2.1 Hz), 6.10 (1H, dd, J=1.8 and 2.1 Hz), 5.86-5.74 (1H, m), 4.81 (1H, dd, J=9.3 and 13.8 Hz), 4.62 (1H, dd, J=5.1 and 13.8 Hz), 4.08-4.00 (1H, m), 1.69 (3H, d, J=6.9 Hz) and 1.40-1.22 ppm (4H, m).

### Example 32

### 3-Methyl-8-[1-(1H-pyrazol-1-yl)propan-2-yl]-3,8-dihydrobenzo[1,2-d:4,5-d′]bis[1,2,3]triazine-4,9-dione

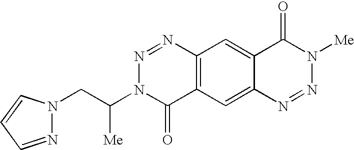

Prepared from intermediates 28 and 9 and 10 using previously described procedures. The resulting white solid had the following properties: mp=201-203° C.; ¹H NMR (300 MHz, CDCl₃) δ 9.11 (1H, s), 9.04 (1H, s), 7.37 (1H, d, J=1.2 Hz), 7.30 (1H, d, J=2.1 Hz), 6.10 (1H, dd, J=1.2 and 2.1 Hz), 5.86-5.74 (1H, m), 4.81 (1H, dd, J=9.3 and 14.1 Hz), 4.62 (1H, dd, J=5.1 and 14.1 Hz), 4.12 (3H, s) and 1.69 ppm (3H, d, J=6.9 Hz).

### Example 33

### (2R)-2-(8-Cyclopropyl-4,9-dioxo-8,9-dihydrobenzo[1,2-d:4,5-d′]bis[1,2,3]triazin-3(4H)-yl)propyl thiocyanate

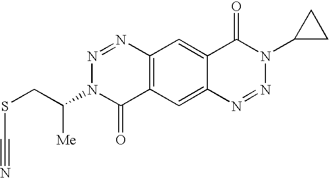

Prepared from intermediate 29 and intermediates 7 and 8 following the procedure in example 1. The resulting white solid had the following properties: mp=178-180° C., ¹H NMR (300 MHz, CDCl₃) δ 9.18 (1H, s), 9.14 (1H, s), 5.73-5.62 (1H, m), 4.10-4.03 (1H, m), 3.74 (1H, dd, J=9.0 and 14.4 Hz), 3.50 (1H, dd, J=4.8 and 14.4 Hz), 1.80 (3H, d, J=6.6 Hz) and 1.45-1.25 ppm (4H, m).

### Example 34

### 3-But-2-yn-1-yl-8-[(2R)-1-(2H-tetrazol-2-yl)propan-2-yl]-3,8-dihydrobenzo[1,2-d:4,5-d′]bis[1,2,3]triazine-4,9-dione

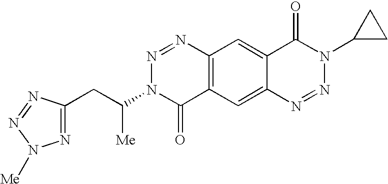

Prepared from intermediate 30 using previously described procedures. The resulting white solid had the following properties: mp=132-134° C.; ¹H NMR (300 MHz, CDCl₃) δ 9.14 (1H, s), 9.08 (1H, s), 5.82-5.68 (1H, m), 4.21 (3H, s), 4.20-4.00 (1H, m), 3.71 (1H, dd, J=8.4 and 15.0 Hz), 3.54 (1H, dd, J=6.0 and 15.0 Hz), 1.75 (3H, d, J=6.9 Hz) and 1.42-1.20 ppm (4H, m).

### Example 35

### N-[(2R)-2-(8-Cyclopropyl-4,9-dioxo-8,9-dihydrobenzo[1,2-d:4,5-d′]bis[1,2,3]triazin-3(4H)-yl)propyl]methanesulfonamide

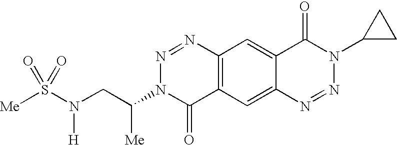

The title compound was prepared from intermediate 32 using the procedure of example 1. The resulting white solid had the following properties: mp=195-197° C., ¹H NMR (300 MHz, CDCl₃) δ 9.13 (1H, s), 9.09 (1H, s), 5.53-5.42 (1H, m), 4.87 (1H, t, J=6.0 Hz), 4.13-4.03 (1H, m), 3.88-3.64 (2H, m), 2.94 (3H, s), 1.66 (3H, d, J=6.3 Hz) and 1.44-1.24 ppm (4H, m).

### Example 36

### N-[(2R)-2-(8-Cyclopropyl-4,9-dioxo-8,9-dihydrobenzo[1,2-d:4,5-d′]bis[1,2,3]triazin-3(4H)-yl)propyl]-N-methylmethanesulfonamide

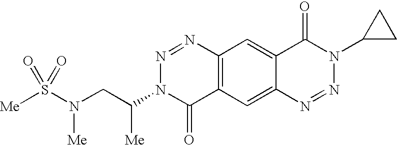

Title compound prepared from intermediate 33 using the procedures of example 1. The resulting white solid had the following properties: mp=170-172° C.; ¹H NMR (300 MHz, CDCl₃) δ 9.15 (1H, s), 9.14 (1H, s), 5.66-5.54 (1H, m), 4.10-4.00 (1H, m), 3.89 (1H, dd, J=9.3 and 14.1 Hz), 3.40 (1H, dd, J=4.5 and 14.1 Hz), 3.95 (3H, s), 2.75 (3H, s), 1.69 (3H, d, J=6.6 Hz) and 1.42-1.22 ppm (4H, m).

### Example 37

### 3-Cyclopropyl-8-[(2S)-1-(3-fluorophenyl)but-3-yn-2-yl]-3,8-dihydrobenzo[1,2-d:4,5-d]′bis[1,2,3]triazine-4,9-dione

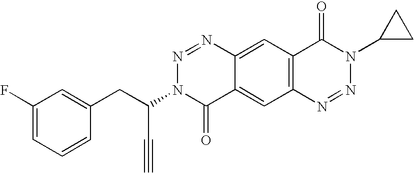

Title compound prepared from intermediates 19 and 34 following the procedure in example 16. The resulting white solid had the following properties: mp=188-190° C.; ¹H NMR (300 MHz, CDCl₃) δ 9.14 (1H, s), 9.04 (1H, s), 7.26-6.85 (4H, m), 6.17 (1H, dt, J=2.1 and 7.5 Hz), 4.09-4.01 (1H, m), 3.55 (2H, d, J=7.5 Hz), 2.53 (1H, d, J=2.1 Hz) and 1.43-1.23 ppm (4H, m).

### Example 38

### 3-[(2R)-1-(2H-Tetrazol-2-yl)propan-2-yl]-8-[(2R)-1-(2H-1,2,3-triazol-2-yl)propan-2-yl]-3,8-dihydrobenzo[1,2-d:4,5-d′]bis[1,2,3]triazine-4,9-dione

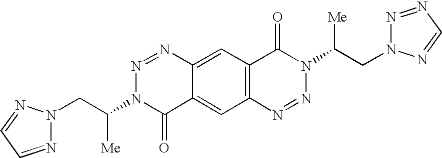

The title compound was prepared from intermediates 1 and 37 following the procedure in example 1. The resulting white solid had the following properties: mp=139-140° C., ¹H NMR (300 MHz, CDCl₃) δ 9.05 (1H, s), 9.04 (1H, s), 8.43 (1H, s), 7.49 (2H, s), 5.90-5.74 (1H, m), 5.37 (1H, dd, J=9.0 and 14.1 Hz), 5.15 (1H, dd, J=4.2 and 14.1 Hz), 5.11 (1H, dd, J=9.0 and 13.8 Hz), 4.93 (1H, dd, J=4.5 and 13.8 Hz), 1.81 (3H, d, J=7.2 Hz) and 1.76 ppm (3H, d, J=7.2 Hz).

### Example 39

### 3-[(2R)-1-(2H-Tetrazol-2-yl)propan-2-yl]-8-[(2R)-1-(1H-1,2,3-triazol-1-yl)propan-2-yl]-3,8-dihydrobenzo[1,2-d:4,5-d′]bis[1,2,3]triazine-4,9-dione

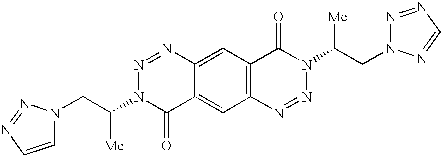

The title compound was prepared from intermediates 1 and 38 following the procedure in example 1 and isolated as a foam; ¹H NMR (300 MHz, CDCl₃) δ 9.05 (2H, s), 8.42 (1H, s), 7.63 (1H, s), 7.56 (1H, s), 5.90-5.76 (1H, m), 5.37 (1H, dd, J=9.0 and 14.1 Hz), 5.17 (1H, dd, J=4.8 and 14.1 Hz), 5.13 (1H, dd, J=9.3 and 14.1 Hz), 4.88 (1H, dd, J=5.1 and 14.1 Hz), 1.82 (3H, d, J=7.2 Hz) and 1.77 ppm (3H, d, J=6.9 Hz).

### Examples 40 and 41

### 3-[(2R)-1-(2H-Tetrazol-2-yl)propan-2-yl]-8-[(2S)-1-(2H-tetrazol-2-yl)propan-2-yl]-3,8-dihydrobenzo[1,2-d:4,5-d′]bis[1,2,3]triazine-4,9-dione and 3-[(2R)-1-(2H-Tetrazol-2-yl)propan-2-yl]-8-[(2S)-1-(1H-tetrazol-1-yl)propan-2-yl]-3,8-dihydrobenzo[1,2-d:4,5-d′]bis[1,2,3]triazine-4,9-dione

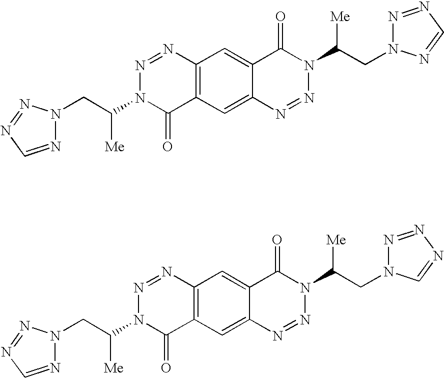

Intermediate 39 (2.0 g, 7.93 mmol) was dissolved in 200 ml of THF/CHCl₃ (4:1), cooled to 0° C. (ice bath) and a solution of NaNO₂ (1.2 g) in of H₂O (30 ml) was added and stirred fast. Conc. HCl (0.9 ml) was slowly added and stirred for 10 minutes. (S)-(+)-2-Amino-1-propanol (2.5 g) and a cooled solution of TEA (30 ml) in THF (10 ml) was added and stirred at 0° C. to RT for 4-5 hours. The mixture was diluted with water (100 ml), extracted with ethyl acetate (3×100 ml), washed with satd. NaHCO₃ (100 ml) and dried over Na₂SO₄ and concentrated, which afforded a dark brown solid. The crude solid was purified using flash chromatography with hexane/ethyl acetate (4:1) to hexane/ethyl acetate (3:2) to ethyl acetate. The solvents were evaporated and the product was triturated from MTBE to obtain a brown solid. The triazinone was dissolved in THF/MeOH (100 ml, 1:1). 30 ml of 2M NaOH was added and stirred at room temperature for 1 h. Conc. HCl was added to the reaction mixture (→pH 2) and the solvent evaporated. The mixture was dissolved in DMF (50 ml) and the solvent evaporated to remove the water. The material was dissolved in DMF (50 ml), HOBT (715 mg, 5.30 mmol), (2R)-1-(2H-tetrazol-2-yl)-2-aminopropane.HCl (1.59 g, 1.5 eq) were added and the solvent evaporated. The residue was dissolved in DMF (100 ml), DMAP (650 mg, 5.30 mmol), TEA (5 ml) and EDCI (3.03 g, 3 eq) were added and stirred at 60° C. for 4.5 hours. The solvent was evaporated, ethyl acetate (150 ml) was added and water (100 ml) and 2N HCl (pH→2) and the aqueous was re-extracted with ethyl acetate (2×100 ml). The organic phase was washed with saturated sodium bicarbonate solution (100 ml), dried over MgSO₄ and concentrated. The material was dissolved in DMF (100 ml), acetic acid (2 ml) and isoamyl nitrite (5 ml) was added and stirred at room temperature overnight. The solvents were evaporated and the product was purified using flash chromatography with hexane/ethyl:acetate (7:3) to 1:1 hexane/ethyl acetate to CHCl₃/ethyl acetate (1:4). The solvents were evaporated to obtain a yellow solid (700 mg).

To a solution of the preceding material (700 mg, 1.82 mmol), tetrazole (200 mg, 1.5 eq), diphenyl-2-pyridylphosphine (720 mg, 1.5 eq) in dry THF (60 ml) was added DIAD (0.6 ml) in dry THF (5 ml) dropwise under N₂. The mixture was stirred at room temperature for 18 hours. The solvent was evaporated and the residue diluted with chloroform (100 ml), washed with 6N HCl (150 ml), dried (Na₂SO₄) and concentrated to obtain a yellow oil. The oil was purified using flash chromatography (100 g silica gel, chloroform, then 40% ethyl acetate in chloroform to 1:1 ethyl acetate/chloroform and 4% methanol in 1:1 ethyl acetate/chloroform. The product fractions were concentrated and triturated from Hexane/MTBE to give 3-[(2R)-1-(2H-tetrazol-2-yl)propan-2-yl]-8-[(2S)-1-(2H-tetrazol-2-yl)propan-2-yl]-3,8-dihydrobenzo[1,2-d:4,5-d′]bis[1,2,3]triazine-4,9-dione; mp=223-226° C.; ¹H NMR (300 MHz, CDCl₃) δ 9.05 (2H, s), 8.40 (2H, s), 5.90-5.78 (2H, m), 5.36 (2H, dd, J=9.3 and 14.1 Hz), 5.15 (2H, dd, J=4.2 and 14.1 Hz) and 1.81 ppm (6H, d, J=7.2 Hz).

and 3-[(2R)-1-(2H-tetrazol-2-yl)propan-2-yl]-8-[(2S)-1-(1H-tetrazol-1-yl)propan-2-yl]-3,8-dihydrobenzo[1,2-d:4,5-d′]bis[1,2,3]triazine-4,9-dione; mp=204-206° C.; ¹H NMR (300 MHz, CDCl₃) δ 9.05 (2H, s), 8.61 (1H, s), 8.39 (1H, s), 5.90-5.78 (2H, m), 5.34 (1H, dd, J=8.7 and 13.8 Hz), 5.26-5.12 (2H, m), 4.91 (1H, dd, J=4.8 and 14.1 Hz), 1.82 (3H, d, J=6.9 Hz) and 1.77 ppm (3H, d, J=6.6 Hz).

### Example 42

### 3-Methoxy-8-[(2R)-1-(2H-tetrazol-2-yl)propan-2-yl]-3,8-dihydrobenzo[1,2-d:4,5-d′]bis[1,2,3]triazine-4,9-dione

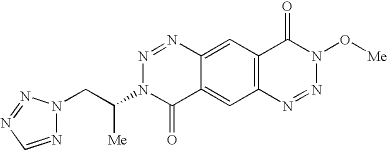

The title compound was prepared from intermediate 40 and methoxylamine hydrochloride following the procedure in example 1. The resulting material had the following properties: mp=227-231° C. (degradation), ¹H NMR (300 MHz, CDCl₃) δ 9.18 (1H, s), 9.10 (1H, s), 8.41 (1H, s), 5.90-5.78 (1H, m), 5.36 (1H, dd, J=9.3 and 14.1 Hz), 5.16 (1H, dd, J=4.5 and 14.1 Hz), 4.33 (3H, s) and 1.82 (3H, d, J=7.2 Hz).

### Example 43

### 3-(3-Methoxypropyl)-8-[(2R)-1-(2H-tetrazol-2-yl)propan-2-yl]-3,8-dihydrobenzo[1,2-d:4,5-e]bis[1,2,3]triazine-4,9-dione

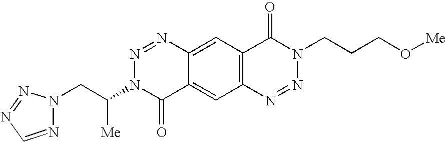

The title compound was prepared from intermediate 40 and 3-methoxypropylamine following the procedure in example 1; mp=164-166° C., ¹H NMR (300 MHz, CDCl₃) δ 9.14 (1H, s), 9.05 (1H, s), 8.41 (1H, s), 5.90-5.78 (1H, m), 5.37 (1H, dd, J=8.7 and 13.8 Hz), 5.16 (1H, dd, J=4.5 and 13.8 Hz), 4.65 (2H, t, J=7.2 Hz), 3.53 (2H, t, J=6.0 Hz), 3.31 (3H, s), 2-27-2.18 (2H, m) and 1.81 (3H, d, J=7.2 Hz).

### Example 44

### 3-Prop-2-en-1-yl-8-[(2R)-1-(2H-tetrazol-2-yl)propan-2-yl]-3,8-dihydrobenzo[1,2-d:4,5-d′]bis[1,2,3]triazine-4,9-dione

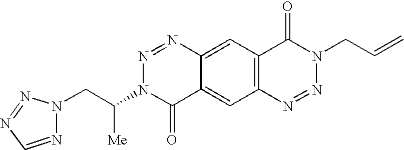

The title compound was prepared from intermediate 40 and allylamine following the procedure in example 1; mp=159-161° C., ¹H NMR (300 MHz, CDCl₃) δ 9.14 (1H, s), 9.06 (1H, s), 8.41 (1H, s), 6.18-6.05 (1H, m), 5.90-5.78 (1H, m), 5.46-5.10 (6H, m) and 1.81 (3H, d, J=6.9 Hz).

### Example 45

### (3R)-3-(8-Cyclopropyl-4,9-dioxo-8,9-dihydrobenzo[1,2-d:4,5-d′]bis[1,2,3]triazin-3(4H)-yl)butanenitrile

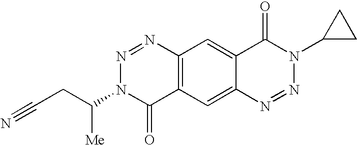

The title compound was prepared from intermediate 42 using previously described procedures; mp=240-242° C.; ¹H NMR (300 MHz, CDCl₃) δ 9.17 (1H, s), 9.12 (1H, s), 5.70-5.58 (1H, m), 4.10-4.02 (1H, m), 3.20 (1H, dd, J=8.1 and 16.8 Hz), 3.04 (1H, dd, J=6.6 and 16.8 Hz), 1.78 (3H, dd, J=6.9 Hz) and 1.42-1.25 ppm (4H, m).

### Example 46

### (3R)-3-{4,9-Dioxo-8-[(2R)-1-(2H-1,2,3-triazol-2-yl)propan-2-yl]-8,9-dihydrobenzo[1,2-d:4,5-d′]bis[1,2,3]triazin-3(41)-yl}butanenitrile

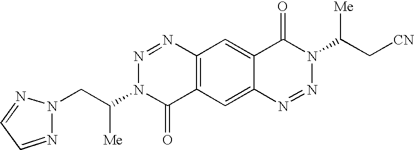

The title compound was prepared from intermediates 37 and 42 using previously described procedures; mp=164-166° C.; ¹H NMR (300 MHz, CDCl₃) δ 9.13 (1H, s), 9.08 (1H, s), 7.47 (2H, s), 5.85-5.75 (1H, m), 5.70-5.58 (1H, m), 5.10 (1H, dd, J=8.7 and 14.1 Hz), 4.94 (1H, dd, J=4.8 and 14.1 Hz), 3.25-3.00 (2H, m) and 1.77 ppm (6H, d, J=6.9 Hz).

### Example 47

### (3R)-3-{4,9-Dioxo-8-[(2R)-1-(2H-tetrazol-2-yl)propan-2-yl]-8,9-dihydrobenzo[1,2-d:4,5-d′]bis[1,2,3]triazin-3(4H)-yl}butanenitrile

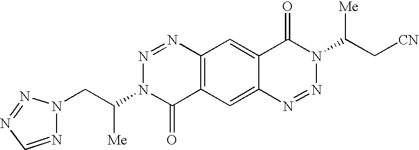

The title compound was prepared from intermediates 40 and 42 using previously described procedures; mp=105-108° C.; ¹H NMR (300 MHz, CDCl₃) δ 9.15 (1H, s), 9.09 (1H, s), 8.40 (1H, s), 5.90-5.77 (1H, m), 5.68-5.56 (1H, m), 5.36 (1H, dd, J=9.0 and 14.1 Hz), 5.17 (1H, dd, J=4.8 and 14.1 Hz), 3.19 (1H, dd, J=7.8 and 16.8 Hz), 3.05 (1H, dd, J=6.6 and 16.8 Hz), 1.82 (3H, d, J=6.9 Hz) and 1.78 ppm (3H, d, J=6.6 Hz).

### Example 48

### 3-But-2-yn-1-yl-8-[(2R)-1-(2H-tetrazol-2-yl)propan-2-yl]-3,8-dihydrobenzo[1,2-d:4,5-d′]bis[1,2,3]triazine-4,9-dione

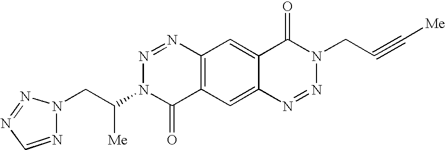

The title compound was prepared from intermediates 40 and but-2-yn-1-amine using the procedure described for example 1; mp=188-190° C.; ¹H NMR (300 MHz, CDCl₃) δ 9.16 (1H, s), 9.06 (1H, s), 8.41 (1H, s), 5.90-5.78 (1H, m), 5.36 (1H, dd, J=9.0 and 14.1 Hz), 5.22 (2H, q, J=2.4 Hz), 5.16 (1H, dd, J=4.5 and 14.1 Hz), 1.84 (3H, t, J=2.4 Hz) and 1.81 ppm (3H, d, J=7.2 Hz).

### Example 49

### 3-But-2-yn-1-yl-8-[(2R)-1-(2H-1,2,3-triazol-2-yl)propan-2-yl]-3,8-dihydrobenzo[1,2-d:4,5-d′]bis[1,2,3]triazine-4,9-dione

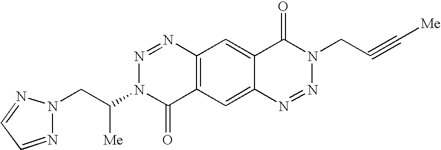

The title compound was prepared from intermediates 37 and but-2-yn-1-amine using the procedure described for example 1; mp=154-156° C.; ¹H NMR (300 MHz, CDCl₃) δ 9.14 (1H, s), 9.06 (1H, s), 7.46 (2H, s), 5.86-5.74 (1H, m), 5.22 (2H, q, J=2.4 Hz), 5.10 (1H, dd, J=8.7 and 13.8 Hz), 4.94 (1H, dd, J=4.5 and 13.8 Hz), 1.84 (3H, t, J=2.4 Hz) and 1.77 (3H, d, J=7.2 Hz).

### Example 50

### 3-Pent-3-yn-2-yl-8-[(2R)-1-(2H-tetrazol-2-yl)propan-2-yl]-3,8-dihydrobenzo[1,2-d:4,5-d′]bis[1,2,3]triazine-4,9-dione

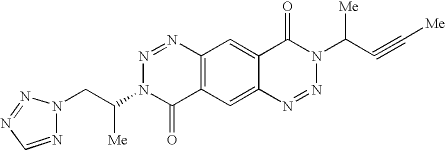

The title compound was prepared from intermediates 40 and pent-3-yn-2-amine using the procedure described for example 1; mp=72-74° C.; ¹H NMR (300 MHz, CDCl₃) δ 9.14 (1H, s), 9.06 (1H, s), 8.40 (1H, s), 6.10-6.00 (1H, m), 5.90-5.78 (1H, m), 5.34 (1H, dd, J=8.1 and 13.8 Hz), 5.15 (1H, dd, J=4.5 and 14.1 Hz), 1.86 (3H, d, J=1.5 Hz), 1.84 (3H, d, J=7.2 Hz) and 1.81 (3H, d, J=6.9 Hz).

### Example 51

### 3,8-Bis[(2R)-1-(2H-1,2,3-triazol-2-yl)propan-2-yl]-3,8-dihydrobenzo[1,2-d:4,5-d′]bis[1,2,3]triazine-4,9-dione

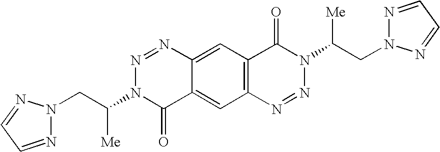

The title compound was prepared from intermediate 25 using the procedure described for example 16; mp: 194-195° C., ¹H NMR (300 MHz, CDCl₃) δ 9.05 (2H, s), 7.50 (4H, s), 5.86-5.75 (2H, m), 5.11 (2H, dd, J=8.7 and 13.8 Hz), 4.94 (2H, dd, J=4.5 and 13.8 Hz) and 1.77 ppm (6H, d, J=6.9 Hz).

### Example 52

### 3,8-Bis[(2R)-1-(1H-1,2,3-triazol-1-yl)propan-2-yl]-3,8-dihydrobenzo[1,2-d:4,5-d′]bis[1,2,3]triazine-4,9-dione

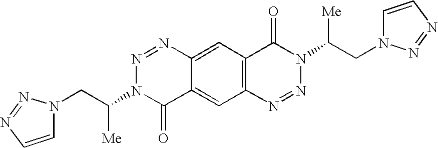

The title compound was prepared from intermediates 26 using the procedure described for example 16; mp: 192-195° C., ¹H NMR (300 MHz, CDCl₃) δ 9.03 (2H, s), 7.62 (2H, s), 7.55 (2H, s), 5.87-5.76 (2H, m), 5.11 (2H, dd, J=9.3 and 14.1 Hz), 4.94 (2H, dd, J=5.1 and 14.1 Hz) and 1.77 ppm (6H, d, J=6.6 Hz).

### Examples 53 and 54

### 3-Cyclopropyl-8-[(2R)-1-(2H-tetrazol-2-yl)propan-2-yl]-3,8-dihydro[1,2,3]triazino[4,5-g]quinazoline-4,9-dione and 3-Cyclopropyl-8-[(2R)-1-(1H-tetrazol-1-yl)propan-2-yl]-3,8-dihydro[1,2,3]triazino[4,5-g]quinazoline-4,9-dione

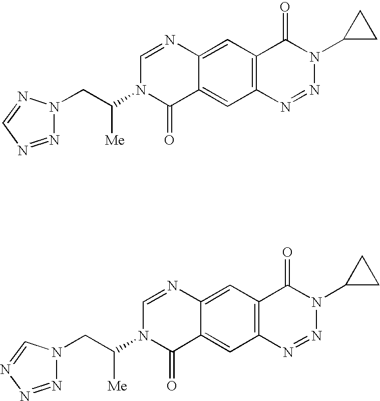

To a stirred suspension of intermediate 43 (8.00 g, 25.5 mmol), tetrazole (7.40 g, 105.7 mmol) and triphenylphosphine (11.00 g, 42.0 mmol) in anhydrous THF (150 ml) was added DIAD (10 ml, 51.4 mmol) in one portion. Stirring under argon was continued at 20° C. for 14 h. The volatiles were removed under reduced pressure, and the residue was subjected to column chromatography using silica gel and chloroform/ethyl acetate/ethanol (50:48:2) as an eluent. The fractions containing the less polar 3-cyclopropyl-8-[(2R)-1-(2H-tetrazol-2-yl)propan-2-yl]-3,8-dihydro[1,2,3]triazino[4,5-g]quinazoline-4,9-dione were combined, the solvent was removed under reduced pressure, and the residue re-crystallized from ethanol; mp=192-194° C. ¹H NMR (300 MHz, CDCl₃) δ 9.05 (1H, s), 8.62 (1H, s), 8.46 (1H, s), 7.81 (1H, s), 5.41 (1H, dd, J=7.8 and 13.8 Hz), 5.24-5.10 (1H, m), 5.08 (1H, dd, J=4.5 and 13.8 Hz), 4.04-3.96 (1H, m), 1.79 (3H, d, J=6.9 Hz) and 1.40-1.20 ppm (4H, m).

Elution of the silica gel column with chloroform/ethyl acetate/ethanol (5:4:1) gave 3-cyclopropyl-8-[(2R)-1-(1H-tetrazol-1-yl)propan-2-yl]-3,8-dihydro[1,2,3]triazino[4,5-g]quinazoline-4,9-dione as the more polar isomer; mp=245-246° C.; ¹H NMR (300 MHz, CDCl₃) δ 9.31 (1H, s), 8.83 (1H, s), 8.51 (1H, s), 8.45 (1H, s), 5.29 (1H, m), 5.17 (1H, dd, 9.3 and 14.1 Hz), 4.99 (1H, dd, J=4.8 and 14.1 Hz), 3.92 (1H, m), 1.68 (3H, d, J=6.9 Hz), and 1.22 (4H, m).

### Example 55

### 8-[(2R)-1-(1H-Benzotriazol-1-yl)propan-2-yl]-3-cyclopropyl-3,8-dihydro[1,2,3]triazino[4,5-g]quinazoline-4,9-dione

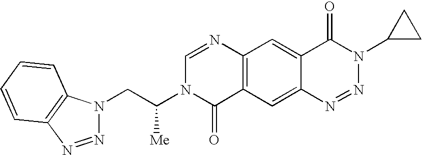

The title compound was prepared from intermediate 43 using the procedure described for examples 53 and 54; mp=103-105° C.; ¹H NMR (300 MHz, CDCl₃) δ 9.10 (1H, s), 8.71 (1H, s), 8.43 (1H, s), 8.00 (1H, d, J=8.1 Hz), 7.44 (3H, m), 5.24 (1H, m), 5.20 (1H, dd, J=7.3 and 10.5 Hz), 4.80 (1H, dd, J=3.7 and 10.5 Hz), 4.02 (1H, m), 1.82 (3H, d, J=7.2 Hz), 1.37 (2H, m), and 1.25 (2H, m).

### Examples 56 and 57

### 3-Cyclopropyl-8-[(2R)-1-(2H-tetrazol-2-yl)butan-2-yl]-3,8-dihydro[1,2,3]triazino[4,5-g]quinazoline-4,9-dione and 3-Cyclopropyl-8-[(2R)-1-(1H-tetrazol-1-yl)butan-2-yl]-3,8-dihydro[1,2,3]triazino[4,5-g]quinazoline-4,9-dione

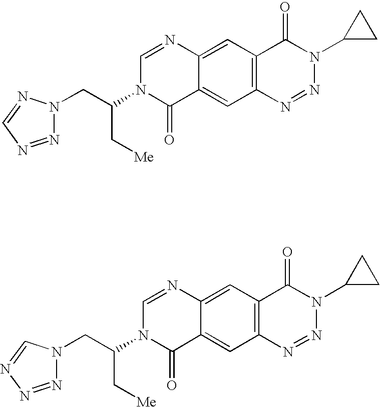

The title compounds were prepared using the procedure for intermediate 43 and using (R)-2-aminobutanol. 3-Cyclopropyl-8-[(2R)-1-(2H-tetrazol-2-yl)butan-2-yl]-3,8-dihydro[1,2,3]triazino[4,5-g]quinazoline-4,9-dione; mp=160-161° C.; ¹H NMR (300 MHz, CDCl₃) δ 9.06 (1H, s), 8.63 (1H, s), 8.46 (1H, s), 7.74 (1H, s), 5.46 (1H, bdd, J=8.8 and 13.9 Hz), 5.07 (1H, dd, J=4.2 and 14.4 Hz), 4.86 (1H, m), 4.00 (1H, m), 2.30 (1H, m), 2.14 (1H, m), 1.34 (2H, m), 1.23 (2H, m), and 1.07 (3H, t, 7.5 Hz).

3-Cycloprop yl-8-[(2R)-1-(1H-tetrazol-1-yl)butan-2-yl]-3,8-dihydro[1,2,3]triazino[4,5-g]quinazoline-4,9-dione; mp=213-214° C.; ¹H NMR (300 MHz, CDCl₃) δ 9.02 (1H, s), 8.64 (1H, s), 8.55 (1H, s), 7.83 (1H, s), 5.34 (1H, dd, J=9.3 and 14.2 Hz), 4.86 (1H, dd, J=5.3 and 14.2 Hz), 4.76 (1H, m), 4.01 (1H, m), 2.34 (1H, m), 2.10 (1H, m), 1.35 (2H, m), 1.23 (2H, m), and 1.05 (3H, t, J=7.5 Hz).

### Examples 58 and 59

### 3-Cyclopropyl-8-[(2R)-1-(2H-1,2,3-triazol-2-yl)butan-2-yl]-3,8-dihydro[1,2,3]triazino[4,5-g]quinazoline-4,9-dione and 3-Cyclopropyl-8-[(2R)-1-(1H-1,2,3-triazol-1-yl)butan-2-yl]-3,8-dihydro[1,2,3]triazino[4,5-g]quinazoline-4,9-dione

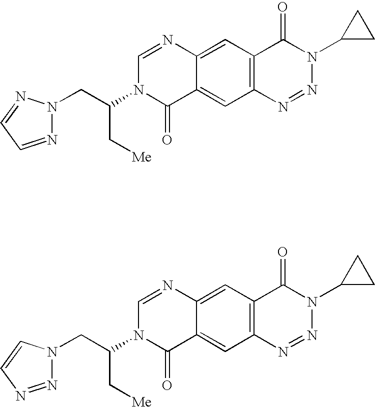

The title compounds were prepared following the procedure for intermediate 43 using (R)-2-aminobutanol and 1,2,3-triazole, as described for examples 53 and 54; 3-Cyclopropyl-8-[(2R)-1-(2H-1,2,3-triazol-2-yl)butan-2-yl]-3,8-dihydro[1,2,3]triazino[4,5-g]quinazoline-4,9-dione; mp=129-130° C.; ¹H NMR (300 MHz, CDCl₃) δ 9.07 (1H, s), 8.60 (1H, s), 7.62 (1H, s), 7.53 (2H, s), 5.15 (1H, m), 4.86 (1H, m), 4.84 (1H, dd, J=4.4 and 14.7 Hz), 4.00 (1H, m), 2.24 (1H, m), 2.11 (1H, m), 1.34 (2H, m), 1.24 (2H, m), and 1.05 (3H, t, J=7.2 Hz).

3-Cyclopropyl-8-[(2R)-1-(1H-1,2,3-triazol-1-yl)butan-2-yl]-3,8-dihydro[1,2,3]triazino[4,5-g]quinazoline-4,9-dione; mp: 215-217° C.; ¹H NMR (300 MHz, CDCl₃) δ 9.04 (1H, s), 8.62 (1H, s), 7.77 (1H, s), 7.60 (1H, d, J=0.9 Hz), 7.46 (1H, d, J=0.9 Hz), 5.16 (1H, dd, J=9.9 and 15.0 Hz), 4.85 (1H, m), 4.83 (1H, dd, J=4.8 and 15.3 Hz), 4.00 (1H, m), 2.29 (1H, m), 2.11 (1H, m), 1.34 (2H, m), 1.23 (2H, m), and 1.04 (3H, t, J=7.2 Hz).

### Examples 60 and 61

### 3-Prop-2-yn-1-yl-8-[(2R)-1-(2H-tetrazol-2-yl)propan-2-yl]-3,8dihydro[1,2,3]triazino[4,5-g]quinazoline-4,9-dione and 3-Prop-2-yn-1-yl-8-[(2R)-1-(1H-tetrazol-1-yl)propan-2-yl]-3,8-dihydro[1,2,3]triazino[4,5-g]quinazoline-4,9-dione

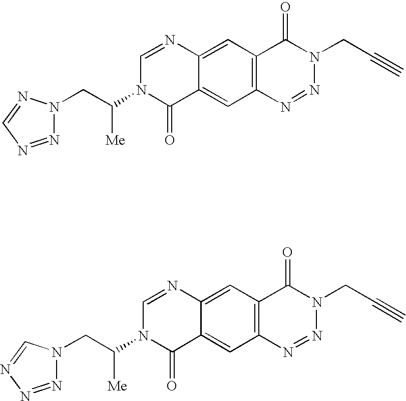

The title compounds were prepared from propargyl amine using the procedures described for examples 53 and 54. 3-Prop-2-yn-1-yl-8-[(2R)-1-(2H-tetrazol-2-yl)propan-2-yl]-3,8dihydro[1,2,3]triazino[4,5-g]quinazoline-4,9-dione; mp=222-223° C.; ¹H NMR (300 MHz, CDCl₃) δ 9.10 (1H, s), 8.64 (1H, s), 8.46 (1, s) 7.81 (1H, s), 5.41 (1H, dd, J=8.1 and 13.9 Hz), 5.23 (2H, d, J=2.6 Hz), 5.18 (1H, m), 5.08 (1H, dd, J=4.4 and 13.9 Hz), 2.41 (1H, t, J=2.6 Hz), and 1.80 (3H, d, J=7.0 Hz).

3-Prop-2-yn-1-yl-8-[(2R)-1-(1H-tetrazol-1-yl)propan-2-yl]-3,8-dihydro[1,2,3]triazino[4,5-g]quinazoline-4,9-dione; mp: 218-219° C.; ¹H NMR. (300 MHz, CDCl₃) δ 9.07 (1H, s), 8.65 (1H, s), 8.57 (1H, s), 7.92 (1H, s), 5.29 (1H, dd, J=8.8 and 14.3 Hz), 5.24 (2H, d, J=2.2 Hz), 5.05 (1H, m), 4.85 (1H, dd, J=5.1 and 14.3 Hz), 2.41 (1H, t, J=2.6 Hz), and 1.81 (3H, d, J=7.3 Hz).

### Example 62

### 8-[(2R)-1-(2H-Tetrazol-2-yl)propan-2-yl]-3-(1H-1,2,3-triazol-4-ylmethyl)-3,8-dihydro[1,2,3]triazino[4,5-g]quinazoline-4,9-dione

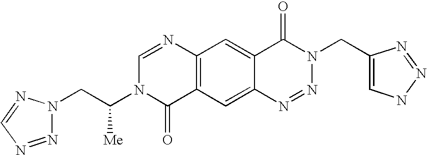

A mixture of 3-prop-2-yn-1-yl-8-[(2R)-1-(2H-tetrazol-2-yl)propan-2-yl]-3,8dihydro[1,2,3]triazino[4,5-g]quinazoline-4,9-dione (185 mg, 0.51 mmol), CuI (120 mg, 0.62 mmol), azidotrimethylsilane (0.13 mL, 1.0 mmol), acetic acid (1 ml) and DMF (3 ml) was heated in a sealed tube at 110° C. for 16 h. The crude product was purified by silica gel chromatography and re-crystallization from ethanol; mp=180-182° C.; ¹H NMR (300 MHz, DMSO-d₆) δ 8.91 (1H, s), 8.56 (1H, s), 8.50 (1H, s), 8.44 (1H, s), 7.70 (1H, bs), 5.73 (2H, s), 5.44 (1H, dd, J=8.8 and 12.8 Hz), 5.37 (1H, m), 5.18 (1H, dd, J=3.7 and 12.8 Hz), and 1.73 (3H, d, J=6.6 Hz).

### Example 63

### 3-Cyclopropyl-8-[2-(2H-tetrazol-2-yl)ethyl]-3,8-dihydro[1,2,3]triazino[4,5-g]quinazoline-4,9-dione

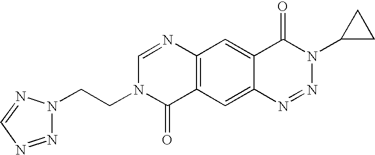

The title compound was prepared from intermediate 20 using the procedures described for examples 43 and 53; mp=194-195° C.; ¹H NMR (300 MHz, CDCl₃) δ 9.09 (1H, s), 8.62 (1H, s), 8.54 (1H, s), 7.70 (1H, s), 5.18 (2H, m), 4.66 (2H, m), 4.00 (1H, m), 1.35 (2H, m), and 1.25 (2H, m).

### Examples 64 and 65

### 3,8-Bis[(2R)-1-(2H-tetrazol-2-yl)propan-2-yl]-3,8-dihydro[1,2,3]triazino[4,5g]quinazoline-4,9-dione and 3-[(2R)-1-(1H-Tetrazol-1-yl)propan-2-yl]-8-[(2R)-1-(2H-tetrazol-2-yl)propan-2-yl]-3,8-dihydro[1,2,3]triazino[4,5-g]quinazoline-4,9-dione

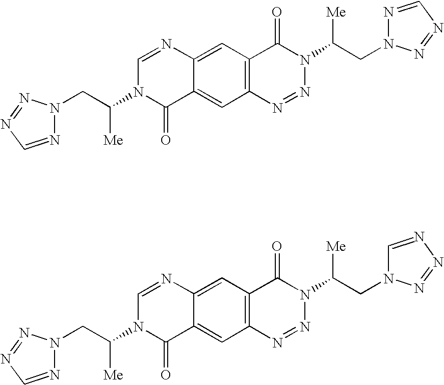

A mixture of intermediate 39 (8.00 g, 28.5 mmol), (R)-2-amino-1-propanol (5.00 g, 66.5 mmol), sodium cyanide (0.49 g, 10 mmol) and ethanol (5 ml) was heated in a stream of argon at 110° C. with distillation of the volatiles. After the distillation ceased, the mixture was heated at the same temperature for an additional 30 min, then poured into 15% NaCl (100 ml), neutralized with 1 N HCl, and extracted with chloroform/isopropanol (4:1, 5×100 ml). The extract was dried over sodium sulfate, concentrated under reduced pressure, and the residue was chromatographed (silica gel, chloroform/ethyl acetate/ethanol (2:1:1) to give the amide as the second fraction. A solution of the amide (2.90 g, 9.05 mmol) in DMF (30 ml) was treated with isopentyl nitrite (4.03 ml) and acetic acid (1.0 ml). The reaction mixture was stirred at 22° C. for 3 days, and the volatiles were removed under reduced pressure to give crude triazinone. A solution of the crude triazinone in dry THF (60 ml) was treated with triphenylphosphine (4.45 g, 17 mmol), tetrazole (1.68 g, 24 mmol) and DIAD (3.6 ml, 18 mmol), and the obtained mixture was stirred at 22° C. for 20 h. The volatiles were removed under reduced pressure, and the residue was chromatographed using a column filled with silica gel and chloroform/ethyl acetate/ethanol (5:4:1) as an eluent. The material from the first fraction was re-crystallized from chloroform (2 ml) to give 3,8-bis[(2R)-1-(2H-tetrazol-2-yl)propan-2-yl]-3,8-dihydro[1,2,3]triazino[4,5g]quinazoline-4,9-dione; mp=184-185° C.; ¹H NMR (300 MHz, CDCl₃) δ 9.08 (1H, s), 8.53 (1H, s), 8.46 (1H, s), 8.40 (1H, s), 7.80 (1H, s), 5.81 (1H, m), 5.41 (1H, dd, J=7.7 and 13.9 Hz), 5.35 (1H, dd, J=8.9 and 13.9 Hz), 5.04-5.20 (3H, m), 1.79 (3H, d, J=6.9 Hz), and 1.78 (3H, d, J=6.9 Hz).

The material from the second fraction was re-crystallized from chloroform to give 3-[(2R)-1-(1H-tetrazol-1-yl)propan-2-yl]-8-[(2R)-1-(2H-tetrazol-2-yl)propan-2-yl]-3,8dihydro[1,2,3]triazino[4,5-g]quinazoline-4,9-dione; mp=168-170° C.; ¹H NMR (300 MHz, CDCl₃) δ 9.07 (1H, s), 8.58 (1H, s), 8.52 (1H, s), 8.45 (1H, s), 7.80 (1H, s), 5.80 (1H, m), 5.42 (1H, dd, J=7.7 and 13.5 Hz), 5.22 (1H, dd, J=9.5 and 14.2 Hz), 5.14 (1H, m), 5.07 (1H, dd, J=4.4 and 13.5 Hz), 4.91 (1H, dd, J=4.8 and 14.2 Hz), 1.80 (3H, d, J=6.9 Hz), and 1.74 (3H, d, J=6.9 Hz).

### Examples 66 and 67

### 8-[(2R)-1-(2H-Tetrazol-2-yl)butan-2-yl]-3-[(2R)-1-(2H-tetrazol-2-yl)propan-2-yl]-3,8-dihydro[1,2,3]triazino[4,5-g]quinazoline-4,9-dione and 8-[(2R)-1-(1H-Tetrazol-1-yl)butan-2-yl]-3-[(2R)-1-(2H-tetrazol-2-yl)propan-2-yl]-3,8-dihydro[1,2,3]triazino[4,5-g]quinazoline-4,9-dione

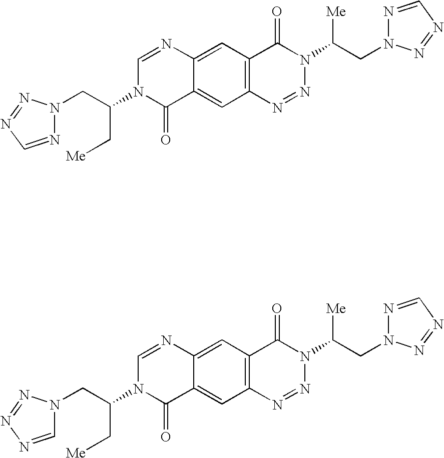

Intermediate 39 (7.0 g, 19.8 mmol) was treated with (R)-2-amino-1-butanol (4.7 ml, 50 mmol), sodium cyanide (245 mg, 5.0 mmol) and methanol (5 ml), and the mixture was heated in a stream of argon with distillation of the solvent. After all the solvent was removed, the mixture was stirred and heated at 100° C. for 30 min. The reaction mixture was diluted with methanol (50 ml), poured into ice/water (100 g), acidified to pH 6 with 6 N HCl, and extracted with chloroform (2×100 ml). The extract was dried over sodium sulfate, the solvent was evaporated under reduced pressure, and the residue was chromatographed (silica gel, chloroform/ethyl acetate/ethanol, 5:4:1) to give the methylester. A solution of the methylester (2.90 g, 9.9 mmol) in methanol (30 ml) was treated with 10 N NaOH (1.5 ml, 15 mmol) and heated at reflux for 15 min. The reaction mixture was acidified with conc. HCl, and the volatiles were removed under reduced pressure. The residue was suspended in DMF (50 ml), and the solvent was thoroughly removed under reduced pressure. A solution of the obtained acid in dry DMF (50 ml) was treated with triethylamine (1.68 ml, 12 mmol), DMAP (1.48 g, 12 mmol), 1-hydroxybenzotriazole hydrate (0.66 g, 5 mmol), and intermediate 1 (1.82 g, 14.3 mmol), and the mixture was stirred under argon and heated at 40° C. for 20 h. The solvent was evaporated under reduced pressure. The residue was dissolved in chloroform (100 ml), poured into water (100 ml), and acidified to pH 5. The chloroform phase was separated, and the aqueous phase was washed with an additional portion of chloroform (50 ml). The chloroform solutions were dried over sodium sulfate, and the solvent was removed under reduce pressure to give crude amide. The crude amide was dissolved in DMF (40 ml) and treated with isobutyl nitrite (10 ml, 84 mmol) and acetic acid (1 ml). The mixture was stirred at 20° C. for 2 days, the solvent was removed under reduced pressure, and the residue was subjected to column chromatography using silica gel and chloroform/ethyl acetate/ethanol/triethylamine (50:45:3:2) as an eluent to give the alcohol. A solution of the alcohol (695 mg, 1.27 mmol), PPh₃ (525 mg, 2.0 mmol), tetrazole (216 mg, 3.0 mmol) and DIAD (0.79 ml, 4.0 mmol) in THF (10 ml) was stirred under argon at r. t. for 3 days. The volatiles were removed under reduced pressure, and the residue was chromatographed. The material from the less polar fraction was re-crystallized from ethanol to give 8-[(2R)-1-(2H-tetrazol-2-yl)butan-2-yl]-3-[(2R)-1-(2H-tetrazol-2-yl)propan-2-yl]-3,8dihydro[1,2,3]triazino[4,5-g]quinazoline-4,9-dione; mp=80-82° C.; ¹H NMR δ 7.08 (1H, s), 8.54 (1H, s), 8.46 (1H, s), 8.41 (1H, s), 7.73 (1H, s), 5.82 (1H, m), 5.47 (1H, dd, J=8.3 and 13.7 Hz), 5.35 (1H, dd, J=8.8 and 13.9 Hz), 5.14 (1H, dd, J=4.6 and 13.9 Hz), 5.07 (1H, dd, J=3.9 and 14.1 Hz), 4.85 (1H, bin), 2.31 (1H, m), 2.12 (1H, m), 1.78 (3H, d, J=6.9 Hz), and 1.07 (3H, t, J=7.5 Hz).

The material from the more polar fraction was re-crystallized from ethanol to give 8-[(2R)-1-(1H-tetrazol-1-yl)butan-2-yl]-3-[(2R)-1-(2H-tetrazol-2-yl)propan-2-yl]-3,8-dihydro[1,2,3]triazino[4,5-g]quinazoline-4,9-dione; mp=198-200° C.; ¹H NMR (300 MHz, CDCl₃) δ 9.05 (1H, s), 8.55 (2H, s), 8.40 (1H, s) 7.83 (1H, s), 5.82 (1H, m), 8.34 (2H, m), 5.15 (1H, dd, J=4.6 and 13.9 Hz), 4.86 (1H, dd, J=4.9 and 14.2 Hz), 4.74 (1H, m), 2.34 (1H, m), 2.20 (1H, m), 1.78 (3H, d, J=6.9 Hz), and 1.05 (3H, t, J=7.5 Hz).

### Examples 68 and 69

### 8-[(2R)-1-(5-Methyl-2H-tetrazol-2-yl)butan-2-yl]-3-[(2R)-1-(2H-tetrazol-2-yl)propan-2-yl]-3,8-dihydro[1,2,3]triazino[4,5-g]quinazoline-4,9-dione and 8-[(2R)-1-(5-Methyl-1H-tetrazol-1-yl)butan-2-yl]-3-[(2R)-1-(2H-tetrazol-2-yl)propan-2-yl]-3,8dihydro[1,2,3]triazino[4,5-g]quinazoline-4,9-dione

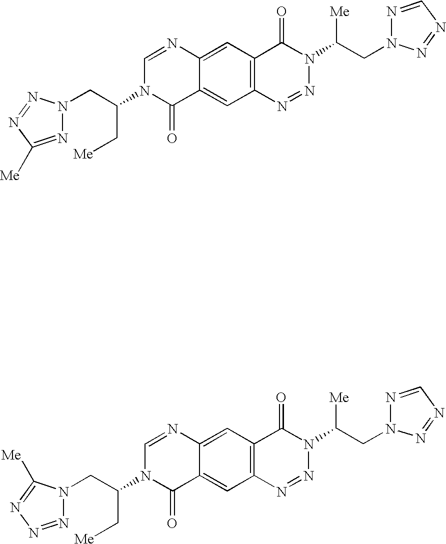

The title compounds were prepared using the procedures described for examples 66 and 67, and 5-methyltetrazole. 8-[(2R)-1-(5-Methyl-2H-tetrazol-2-yl)butan-2-yl]-3-[(2R)-1-(2H-tetrazol-2-yl)propan-2-yl]-3,8-dihydro[1,2,3]triazino[4,5-g]quinazoline-4,9-dione; mp=140-141° C.; ¹H NMR (300 MHz, CDCl₃) δ 9.09 (1H, s), 8.54 (1H, s), 8.41 (1H, s), 7.74 (1H, s), 5.82 (1H, m), 5.35 (2H, m), 5.14 (1H, dd, J=4.6 and 13.9 Hz), 4.96 (1H, dd, J=4.2 and 14.2 Hz), 4.83 (1H, m), 2.46 (3H, s), 2.29 (1H, m), 2.10 (1H, m), 1.78 (3H, d, J=6.9 Hz), and 1.06 (3H, t, J=7.5 Hz).

8-[(2R)-1-(5-Methyl-1H-tetrazol-1-yl)butan-2-yl]-3-[(2R)-1-(2H-tetrazol-2-yl)propan-2-yl]-3,8dihydro[1,2,3]triazino[4,5-g]quinazoline-4,9-dione; mp=238-240° C.; ¹H NMR (300 MHz, CDCl₃) δ 9.02 (1H, s), 8.55 (1H, s), 8.41 (1H, s), 7.90 (1H, s), 5.81 (1H, m), 5.35 (1H, dd, J=8.8 and 13.9 Hz), 5.15 (2H, m), 4.79 (1H, m), 4.63 (1H, dd, J=5.1 and 14.2 Hz), 2.50 (3H, s), 2.36 (1H, m), 2.10 (1H, m), 1.78 (3H, d, J=7.2 Hz), and 1.05 (3H, t, J=7.5 Hz).

### Examples 70 and 71

### 3,8-Bis[(2R)-1-(2H-tetrazol-2-yl)butan-2-yl]-3,8-dihydro[1,2,3]triazino[4,5-g]quinazoline-4,9-dione and 3,8-Bis[(2R)-1-(1H-tetrazol-1-yl)butan-2-yl]-3,8dihydro[1,2,3]triazino[4,5-g]quinazoline-4,9-dione

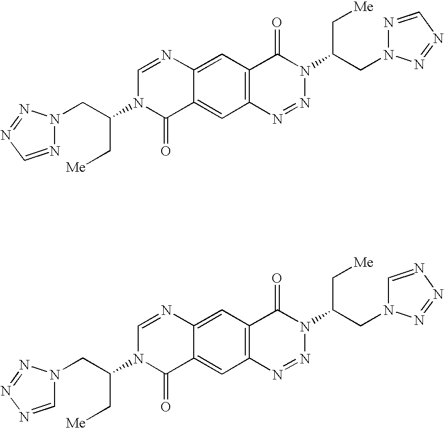

A mixture of intermediate 39 (4.3 g, 12.2 mmol), (R)-2-aminobutanol (2.8 ml, 30 mmol), NaCN (122 mg, 2.5 mmol), and methanol (5 ml) was stirred and heated in a stream of argon with distillation of volatiles. After all the solvent distilled off, heating was continued at 100° C. for an additional period of 30 min. The reaction mixture was diluted with methanol (50 ml), poured into ice/water (100 g) and extracted with chloroform (2×100 ml). The extract was dried over sodium sulfate, the solvent was removed under reduced pressure, and the residue was chromatographed using ethyl acetate/ethanol/acetic acid (95:3:2) as an eluent. The second fraction gave the desired amide. A solution of the amide (1.00 g, 2.87 mmol) in DMF (40 ml) was treated with isobutyl nitrite (5 ml, 42 mmol), and acetic acid (0.5 ml). The mixture was stirred at room temperature for 3 days. The volatiles were removed under reduced pressure. A solution of the residue in THF (20 ml) was treated with tetrazole (560 mg, 8.0 mmol), triphenylphosphine (2.10 g, 8.0 mmol) and di-tert-butylazodicarboxylate (2.30 g, 10 mmol). The reaction mixture was stirred at room temperature for 10 days. The solvent was removed under reduced pressure, and the residue was chromatographed using chloroform/ethyl acetate/ethanol/triethylamine (50:47:2:1) as an eluent. The fractions containing the less polar isomer were identified as being 3,8-bis[(2R)-1-(2H-tetrazol-2-yl)butan-2-yl]-3,8-dihydro[1,2,3]triazino[4,5-g]quinazoline-4,9-dione and the product was re-crystallized from ethanol; mp=80-81° C.; ¹H NMR (300 MHz, CDCl₃) δ 9.09 (1H, s), 8.54 (1H, s), 8.47 (1H, s), 8.40 (1H, s), 7.74 (1H, s), 5.61 (1H, m), 5.48 (1H, dd, J=8.4 and 13.9 Hz), 5.16 (1H, dd, J=9.0 and 13.9 Hz), 5.16 (1H, dd, J=4.2 and 13.9 Hz), 5.08 (1H, dd, J=4.4 and 14.2 Hz), 4.85 (1H, m), 2.32 (2H, m), 2.15 (2H, m), 1.08 (3H, t, J=7.5 Hz), and 1.03 (3H, t, J=7.2 Hz).

The fractions containing the more polar product were combined, the solvent was removed under reduced pressure, and the residue was re-crystallized from ethanol to give 3,8-bis[(2R)-1-(1H-tetrazol-1-yl)butan-2-yl]-3,8dihydro[1,2,3]triazino[4,5-g]quinazoline-4,9-dione; mp=80-81° C.; ¹H NMR (300 MHz, CDCl₃) δ 9.09 (1H, s), 8.54 (1H, s), 8.47 (1H, s), 8.40 (1H, s), 7.74 (1H, s), 5.61 (1H, m), 5.48 (1H, dd, J=8.4 and 13.9 Hz), 5.16 (1H, dd, J=9.0 and 13.9 Hz), 5.16 (1H, dd, J=4.2 and 13.9 Hz), 5.08 (1H, dd, J=4.4 and 14.2 Hz), 4.85 (1H, m), 2.32 (2H, m), 2.15 (2H, m), 1.08 (3H, t, J=7.5 Hz), and 1.03 (3H, t, J=7.2 Hz).

### Examples 72 and 73

### 3,8-Bis[(2R)-1-(2H-tetrazol-2-yl)butan-2-yl]-3,8-dihydropyrimido[4,5-g]quinazoline-4,9-dione and 3-[(2R)-1-Hydroxybutan-2-yl]-8-[(2R)-1-(2H-tetrazol-2-yl)butan-2-yl]-3,8-dihydropyrimido[4,5-g]quinazoline-4,9-dione

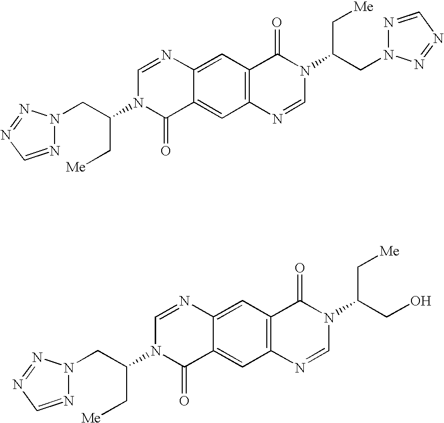

A mixture of formic acid (10 ml) and acetic anhydride (10 ml) was heated at 80° C. for 1 h. Intermediate 39 (1.50 g, 5.95 mmol) was added, and the solution was heated at 90° C. for 4 h. After cooling, the reaction mixture was diluted with ethyl ether (100 ml) and stored at −10° C. for 2 h. the precipitate was filtered off, washed with ethyl ether (20 ml) and dried in a vacuum desiccator. A mixture of this crude formamide, NaCN (245 mg, 5 mmol), (R)-2-aminoethanol (4.0 mL, 42 mmol), DMF (2 ml) and methanol (3 ml) was heated in a stream of argon at 120-130° C. with distillation of volatiles for 1 h. The reaction mixture was poured into 20% NaCl (100 ml), extracted with chloroform/ethanol (4:1, 5×100 ml), and the extract was dried over sodium sulfate. The solvent was removed under reduced pressure to give crude diol. A mixture of the crude diol, tetrazole (560 mg, 8.0 mmol), triphenylphosphine (1.57 g, 6.0 mmol), DIAD (1.97 ml, 10 mmol), and THF (50 ml) was stirred at 20° C. for 18 h. The solvent was removed under reduced pressure, and the residue was chromatographed using chloroform/ethyl acetate/ethanol (50:30:20) as an eluent. The first fraction eluted was identified as 3,8-bis[(2R)-1-(2H-tetrazol-2-yl)butan-2-yl]-3,8-dihydropyrimido[4,5-g]quinazoline-4,9-dione; mp=177-178° C.; ¹H NMR (300 MHz, CDCl₃) δ 8.58 (2H, s), 8.44 (2H, s), 7.64 (2H, s), 5.44 (2H, dd, J=8.1 and 13.6 Hz), 5.06 (2H, dd, J=4.0 and 13.9 Hz), 4.64 (2H, m), 2.29 (2H, m), 2.08 (2H, m), and 1.05 (6H, t, J=7.5 Hz).

The second fraction was identified as 3-[(2R)-1-hydroxybutan-2-yl]-8-[(2R)-1-(2H-tetrazol-2-yl)butan-2-yl]-3,8-dihydropyrimido[4,5-g]quinazoline-4,9-dione; ¹H NMR (300 MHz, CDCl₃) δ 8.57 (1H, s), 8.55 (1H, s), 8.45 (1H, s), 8.19 (1H, s), 7.64 (1H, s), 5.46 (1H, m), 5.07 (1H, dd, J=4.5 and 14.4 Hz), 4.70-4.92 (2H, m), 3.92-4.16 (2H, m), 2.30 (1H, m), 1.90-2.15 (3H, m), 1.59 (1H, bs), 1.06 (3H, t, J=7.8 Hz), and 0.99 (3H, t, J=7.5 Hz).

### Example 74

### 3-[(2R)-1-(2H-Tetrazol-2-yl)butan-2-yl]-8-[(2R)-1-(4H-1,2,4-triazol-4-yl)butan-2-yl]-3,8-dihydropyrimido[4,5-g]quinazoline-4,9-dione

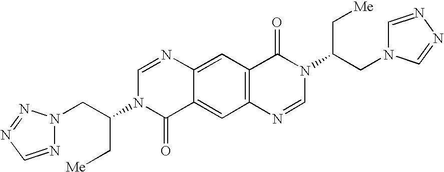

A solution of 3-[(2R)-1-hydroxybutan-2-yl]-8-[(2R)-1-(2H-tetrazol-2-yl)butan-2-yl]-3,8-dihydropyrimido[4,5-g]quinazoline-4,9-dione (400 mg, 1.01 mmol), methyl 1,2,4-triazole-3-carboxylate (1.016 g, 8.0 mmol), triphenylphosphine (1.57 g, 6.0 mmol) and DIAD (1.97 ml, 10.0 mmol) in THF (20 ml) was stirred under argon and heated at 40° C. for 18 h. The solvent was removed under reduced pressure, and the residue was chromatographed. Fractions containing the triazole ester were combined, and the solvent was removed under reduced pressure. A solution of the triazole ester in methanol (30 ml) was treated with 1 N NaOH (2 ml) and stirred at rt. for 30 min. The reaction mixture was acidified to pH 3 with 6 N HCl, and the solvent was evaporated under reduced pressure. The crude product was purified by column chromatography (silica gel, chloroform/ethyl acetate/ethanol/triethylamine, 50:37:8:5) and recrystallization from ethyl ether (5 ml) to give the title product; mp=130-132° C.; ¹H NMR (300 MHz, CDCl₃) δ 8.55 (2H, s), 8.44 (1H, s), 7.93 (1H, s), 7.89 (1H, s), 7.68 (1H, s), 7.65 (1H, s), 5.42 (1H, dd, J=8.1 and 13.9 Hz), 5.07 (1H, dd, J=4.4 and 14.3 Hz), 5.06 (1H, m), 4.85 (1H, m), 4.66 (1H, m), 4.55 (1H, dd, J=4.8 and 13.9 Hz), 2.30 (2H, m), 2.06 (2H, m), 1.05 (3H, t, J=7.5 Hz), and 1.01 (3H, t, J=7.5 Hz).

### Example 75

### 3,8-Bis[(2R)-1-(2H-tetrazol-2-yl)propan-2-yl]-3,8-dihydropyrimido[4,5-g]quinazoline-4,9-dione

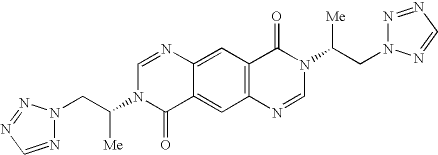

The title compound was prepared using (R)-2-aminopropanol by the procedures described for example 72; mp=255-256° C.; ¹H NMR (300 MHz, CDCl₃+TFA) δ 9.22 (2H, s), 8.87 (2H, s), 8.65 (2H, s), 5.40-5.50 (4H, m), 5.15-5.26 (2H, m), and 1.86 (6H, d, J=6.6 Hz).

### Examples 76 and 77

### 3,8-Bis[(2R)-1-(1H-pyrazol-1-yl)propan-2-yl]-3,8-dihydropyrimido[4,5-g]quinazoline-4,9-dione and 3-[(2R)-1-(4-Chloro-1H-pyrazol-1-yl)propan-2-yl]-8-[(2R)-1-(1H-pyrazol-1-yl)propan-2-yl]-3,8-dihydropyrimido[4,5-g]quinazoline-4,9-dione

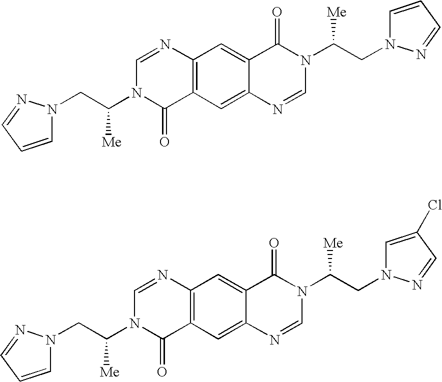

A mixture of formic acid (10 ml) and acetic anhydride (10 ml) was heated at 80° C. for 1 h. Intermediate 39 (1.50 g, 5.95 mmol) was added, and the solution was heated at 90° C. for 4 h. After cooling, the reaction mixture was diluted with ethyl ether (100 ml) and stored at −10° C. for 2 h. the precipitate was filtered off, washed with ethyl ether (20 ml) and dried in a vacuum desiccator. A mixture of the diformamide (1.60 g, 4.5 mmol), (R)-2-aminopropanol (1.55 ml, 20 mmol), NaCN (0.49 g, 10 mmol) and DMF (2 ml) was heated at 130° C. in a stream of argon for 18 h. The obtained crude product was purified by column chromatography (silica gel, ethyl acetate/ethanol, 9:1) and re-crystallization from methanol (50 ml) to give pure diol. To a suspension of the diol (650 mg, 1.96 mmol) in chloroform (10 ml) stirred under argon was added pyridine (1.0 ml, 12.3 mmol) and methane sulfonic anhydride (796 mg, 4.57 mmol). The mixture became warm (40° C.), and the entire solid dissolved. Stirring at room temperature was continued for additional 4 h. The mixture was poured onto crushed ice (50 g), stirred for 1 h, and extracted with chloroform (2×50 ml). The extract was dried over MgSO₄, and the solvent was removed under reduced pressure to give the bis-sulfonate. A solution of the bis-sulfonate (950 mg, 1.75 mmol) and pyrazole (680 mg, 10 mmol) in DMF (5 ml) was stirred under argon and treated with 60% sodium hydride (100 mg, 2.5 mmol). The obtained mixture was heated at 50° C. for 4 h. Methanol (5 ml) and triethylamine (1 ml) were added, and stirring was continued at room temperature overnight. The volatiles were removed under reduced pressure, and the residue was chromatographed (silica gel, chloroform/ethyl acetate/ethanol/triethylamine, 5:3:1:1). The less polar material was identified as 3,8-bis[(2R)-1-(1H-pyrazol-1-yl)propan-2-yl]-3,8-dihydropyrimido[4,5-g]quinazoline-4,9-dione and was re-crystallized from ethanol; mp=239-240° C.; ¹H NMR (300 MHz, CDCl₃) δ 8.55 (2H, s), 7.61 (2H, s), 7.48 (2H, d, J=1.8 Hz), 7.20 (2H, d, J=2.1 Hz), 6.10 (2H, t, J=2.1 Hz), 4.97 (2H, m), 4.80 (2H, dd, J=8.1 and 13.9 Hz), 4.50 (2H, dd, J=4.8 and 13.9 Hz), and 1.71 (6H, d, J=6.9 Hz).

The second fraction gave 3-[(2R)-1-hydroxypropan-2-yl]-8-[(2R)-1-(1H-pyrazol-1-yl)propan-2-yl]-3,8-dihydropyrimido[4,5-g]quinazoline-4,9-dione and this was reacted with chloropyrazole using the procedure described for example 76 to give 3-[(2R)-1-(4-chloro-1H-pyrazol-1-yl)propan-2-yl]-8-[(2R)-1-(1H-pyrazol-1-yl)propan-2-yl]-3,8-dihydropyrimido[4,5-g]quinazoline-4,9-dione; mp=231-232° C.; ¹H NMR (300 MHz, CDCl₃) δ 8.57 (1H, s), 8.55 (1H, s), 7.70 (1H, s), 7.61 (1H, s), 7.49 (1H, d, J=1.5 Hz), 7.40 (1H, s), 7.26 (1H, s), 7.21 (1H, d, J=1.8 Hz), 6.10 (1H, t, J=2.0 Hz), 4.96 (2H, m), 4.79 (2H, m), 4.51 (1H, dd, J=4.8 and 13.9 Hz), 4.43 (1H, dd, J=5.1 and 13.9 Hz), 1.72 (3H, d, J=6.6 Hz), and 1.69 (3H, d, J=6.6 Hz).

### Example 78

### 3,8-Bis[2-(3-fluorophenyl)ethyl]-3,8-dihydropyrimido[4,5-g]quinazoline-4,9-dione

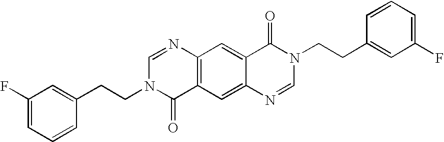

A mixture of formic acid (10 ml) and acetic anhydride (10 ml) was heated at 80° C. for 1 h. Intermediate 39 (1.50 g, 5.95 mmol) was added, and the solution was heated at 90° C. for 4 h. After cooling, the reaction mixture was diluted with ethyl ether (100 ml) and cooled at −10° C. for 2 hour. The resulting precipitate was filtered off, washed with ethyl ether (20 ml) and dried in a vacuum desiccator. A mixture of this diformamide (525 mg, 1.5 mmol) and 3-fluorophenethylamine (1.30 ml, 10 mmol) was heated in a stream of argon at 180° C. for 30 min. The crude product was purified by column chromatography (silica gel, hexanes/ethyl acetate/triethylamine, 50:45:5) and recrystallization from ethanol to give the title compound; mp=250-251° C.; ¹H NMR (300 MHz, CDCl₃) δ 8.65 (2H, s), 7.70 (2H, s), 7.25 (2H, m), 6.93 (6H, m), 4.24 (4H, t, J=7.0 Hz), and 3.13 (4H, t, J=7.1 Hz).

### Example 79

### 3-[(2R)-1-Hydroxypropan-2-yl]-8-[(2R)-1-(2H-tetrazol-2-yl)propan-2-yl]-3,8-dihydrobenzo[1,2-d:4,5-d′]bis[1,2,3]triazine-4,9-dione

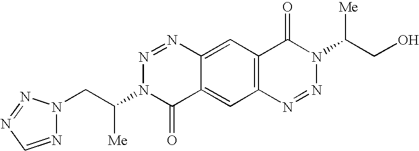

To a stirred solution of intermediate 39 (6.10 g, 21.8 mmol) in THF/chloroform (200+100 ml) was added a solution of sodium nitrite (2.50 g, 36 mmol) in water (60 ml). The vigorously stirred mixture was cooled to 0° C. in an ice/methanol bath and treated with conc. HCl (5.0 ml, 60 mmol) added dropwise. After stirring at 0° C. for an additional period of 15 min, (R)-2-aminopropanol (5.0 g, 66 mmol) followed by triethylamine (13.9 ml, 100 mmol) were added dropwise. The stirred mixture was allowed to warm up within 1 h to 10° C., and was poured onto crushed ice (200 g). The mixture was extracted with chloroform/ethanol (9:1, 3×200 ml), the extract was dried over sodium sulfate, and the solvent was removed under reduced pressure. The residue was triturated with ethanol (75 ml) to give the crystalline triazinone intermediate. A mixture of the triazinone (1.90 g, 6.2 mmol), 10 N NaOH (2.0 ml, 20 mmol) and methanol (50 ml) was heated at reflux for 15 min. The mixture was cooled, acidified by addition of conc. HCl (2.0 ml, 24 mmol), and the volatiles were removed under reduced pressure. The residue was diluted with DMF (50 ml), and the volatiles were thoroughly removed under reduced pressure to give crude aminoacid. The product was directly used in the next step. A solution of crude aminoacid in DMF (50 ml) was stirred under argon and treated with DMAP (1.20 g, 10 mmol), BtOH.H₂O (0.66 g, 5.0 mmol), triethylamine (1.4 ml, 10 mmol), intermediate 1 (1.14 g, 9.0 mmol), and DIC (4.7 ml, 30 mmol). The mixture was stirred and heated at 40° C. for 16 h. The volatiles were removed under reduced pressure, and the residue was chromatographed using ethyl acetate/ethanol/acetic acid (97:2:1) as eluent. A solution of this material (2.20 g, 4.54 mmol) in DMF (30 ml) was treated with isobutyl nitrite (10 ml, 84 mmol) and acetic acid (1.0 ml) and kept at 18° C. for 20 h. The volatiles were removed under reduced pressure, and the residue was chromatographed (silica gel, chloroform/ethyl acetate/ethanol, 50:45:5) to give the title compound; mp=153-154° C.; ¹H NMR (300 MHz, CDCl₃) δ 9.14 (1H, s), 9.05 (1H, s), 8.40 (1H, s), 5.84 (1H, m), 5.44 (1H, m), 5.36 (1H, dd, J=8.8 and 14.3 Hz), 5.16 (1H, dd, J=4.4 and 13.9 Hz), 4.12 (2H, m), 2.02 (1H, dd, J=4.8 and 7.2 Hz), 1.82 (3H, d, J=6.9 Hz), and 1.61 (3H, d, J=6.9 Hz).

### Example 80

### 3,8-Bis[(2R)-1-(2H-tetrazol-2-yl)propan-2-yl]-3,8-dihydrobenzo[1,2-d:4,5-d′]bis[1,2,3]triazine-4,9-dione

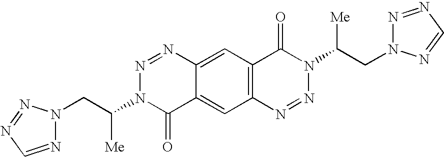

A mixture of 3-[(2R)-1-hydroxypropan-2-yl]-8-[(2R)-1-(2H-tetrazol-2-yl)propan-2-yl]-3,8-dihydrobenzo[1,2-d:4,5-d′]bis[1,2,3]triazine-4,9-dione (1.40 g, 2.73 mmol), triphenyl phosphine (1.05 g, 4.0 mmol), tetrazole (0.42 g, 6.0 mmol), DIAD (1.58 ml, 8.0 mmol), and THF (20 ml) was stirred at 18° C. for 20 h. The volatiles were removed under reduced pressure, and the residue was chromatographed (silica gel, ethyl acetate/ethanol/acetic acid, 97:2:1). Fractions containing the desired material were combined, the solvent was removed under reduced pressure, and the residue was recrystallized from ethanol to give the title compound; mp=64-66° C.; ¹H NMR (300 MHz, CDCl₃) δ 9.04 (2H, s), 8.41 (2H, s), 5.83 (2H, m), 5.36 (2H, dd, J=9.2 and 13.9 Hz), 5.16 (2H, dd, J=4.4 and 13.9 Hz), and 1.81 (6H, d, J=7.2 Hz).

### Examples 81 and 82

### 3-[(2R)-1-(5-Methyl-2H-tetrazol-2-yl)propan-2-yl]-8-[(2R)-1-(2H-tetrazol-2-yl)propan-2-yl]-3,8-dihydrobenz[1,2-d:4,5-d′]bis[1,2,3]triazine-4,9-dione and 3-[(2R)-1-(5-Methyl-1H-tetrazol-1-yl)propan-2-yl]-8-[(2R)-1-(2H-tetrazol-2-yl)propan-2-yl]-3,8-dihydrobenzo[1,2-d:4,5-d′]bis[1,2,3]triazine-4,9-dione

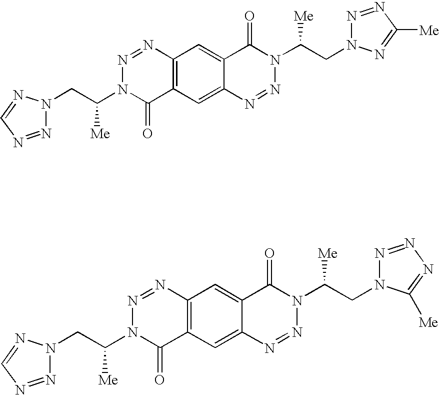

Prepared from 5-methyltetrazole using the procedures described for example 80. The isomers obtained were separated by column chromatography (silica gel, chloroform/ethyl acetate/ethanol, 50:45:5). The less polar isomer identified as 3-[(2R)-1-(5-methyl-2H-tetrazol-2-yl)propan-2-yl]-8-[(2R)-1-(2H-tetrazol-2-yl)propan-2-yl]-3,8-dihydrobenzo[1,2-d:4,5-d′]bis[1,2,3]triazine-4,9-dione had the following properties: mp=66-68° C.; ¹H NMR (300 MHz, CDCl₃) δ 9.06 (1H, s), 9.05 (1H, s), 8.41 (1H, s), 5.82 (2H, m), 5.36 (1H, dd, J=9.2 and 13.9 Hz), 5.26 (1H, dd, J=8.8 and 13.9 Hz), 5.16 (1H, dd, J=4.4 and 13.9 Hz), 5.04 (1H, dd, J=5.7 and 13.9 Hz), 2.44 (3H, s), 1.81 (3H, d, J=6.9 Hz), and 1.78 (3H, d, J=6.9 Hz).

The more polar isomer identified as 3-[(2R)-1-(5-methyl-1H-tetrazol-1-yl)propan-2-yl]-8-[(2R)-1-(2H-tetrazol-2-yl)propan-2-yl]-3,8-dihydrobenzo[1,2-d:4,5-d′]bis[1,2,3]tri azine-4,9-dione had the following properties: mp=81-82° C.; ¹H NMR (300 MHz, CDCl₃) δ 9.05 (1H, s), 9.04 (1H, s), 8.40 (1H, s), 5.82 (2H, m), 5.35 (1H, dd, J=8.8 and 13.9 Hz), 5.16 (1H, dd, J=4.8 and 13.9 Hz), 5.02 (1H, dd, J=9.2 and 14.3 Hz), 4.68 (1H, dd, J=5.3 and 14.3 Hz), 2.44 (3H, s), 1.81 (3H, d, J=6.9 Hz), and 1.78 (3H, d, J=6.9 Hz).

### Example 83

### 3-[(2R)-1-(2H-Tetrazol-2-yl)propan-2-yl]-8-{(2R)-1-[3-(trifluoromethyl)-1H-pyrazol-1-yl]propan-2-yl}-3,8-dihydrobenzo[1,2-d:4,5-d′]bis[1,2,3]triazine-4,9-dione

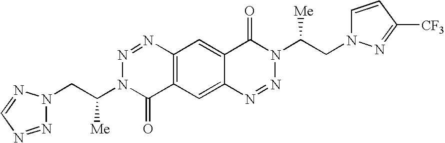

Prepared using 3-(trifluoromethyl)pyrazole and following the procedures in example 80; mp=95-97° C.; ¹H NMR (300 MHz, CDCl₃) δ 9.05 (1H, s), 9.03 (1H, s), 8.40 (1H, s), 7.38 (1H, m), 6.41 (1H, d, J=2.2 Hz), 5.79 (2H, m), 5.35 (1H, dd, J=9.2 and 14.3 Hz), 5.16 (1H, dd, J=4.4 and 13.9 Hz), 4.84 (1H, dd, J=9.2 and 14.3 Hz), 4.65 (1H, dd, J=5.1 and 13.9 Hz), 1.81 (3H, d, J=6.9 Hz), and 1.72 (3H, d, J=6.6 Hz).

### Examples 84 and 85

### 3-[(2R)-1-(2H-Tetrazol-2-yl)butan-2-yl]-8-[(2R)-1-(2H-tetrazol-2-yl)propan-2-yl]-3,8-dihydrobenzo[1,2-d:4,5-d′]bis[1,2,3]triazine-4,9-dione and 3-[(2R)-1-(1H-Tetrazol-1-yl)butan-2-yl]-8-[(2R)-1-(2H-tetrazol-2-yl)propan-2-yl]-3,8-dihydrobenzo[1,2-d:4,5-d′]bis[1,2,3]triazine-4,9-dione

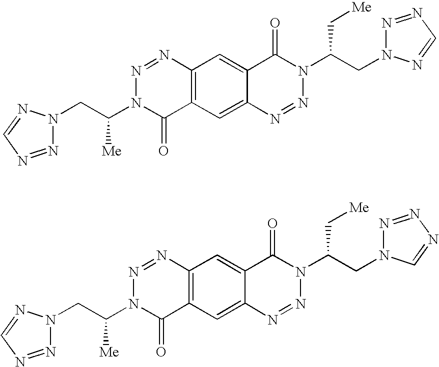

Prepared from (R)-2-aminobutanol using the procedures described for examples 79 and 80. The compounds were separated by column chromatography (silica gel, ethyl acetate/ethanol/acetic acid, 97:2:1). The less polar isomer, identified as 3-[(2R)-1-(2H-tetrazol-2-yl)butan-2-yl]-8-[(2R)-1-(2H-tetrazol-2-yl)propan-2-yl]-3,8-dihydrobenzo[1,2-d:4,5-d′]bis[1,2,3]triazine-4,9-dione had the following properties: mp: 58-60° C.; ¹H NMR (300 MHz, CDCl₃) δ 9.06 (1H, s), 9.05 (1H, s), 8.42 (1H, s), 8.40 (1H, s), 5.83 (1H, m), 5.61 (1H, m), 5.36 (2H, dd, J=9.0 and 14.2 Hz), 5.17 (2H, m), 2.32 (1H, m), 2.18 (1H, m), 1.81 (3H, d, J=6.9 Hz), and 1.05 (3H, t, J=7.5 Hz).

The more polar isomer was purified by recrystallization from ethanol and had the following properties: mp: 146-147° C.; ¹H NMR (300 MHz, CDCl₃) δ 9.07 (1H, s), 9.05 (1H, s), 8.59 (1H, s), 8.41 (1H, s), 5.82 (1H, m), 5.64 (1H, m), 5.36 (1H, dd, J=8.8 and 13.9 Hz), 5.22 (1H, dd, J=10.0 and 14.4 Hz), 5.17 (1H, dd, J=4.4 and 13.9 Hz), 4.94 (1H, dd, J=4.4 and 14.4 Hz), 2.28 (1H, m), 2.12 (1H, m), 1.81 (3H, d, J=6.9 Hz), and 1.04 (3H, t, J=7.5 Hz).

### Example 86

### 3-[(2R)-1-(2H-Tetrazol-2-yl)propan-2-yl]-8-[(2R)-1(2H-1,2,3-triazol-2-yl)butan-2-yl]-3,8-dihydrobenzo[1,2-d:4,5-d′]bis[1,2,3]triazine-4,9-dione

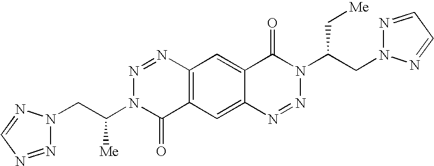

The title compound was prepared using the procedures used to prepare examples 84 and 85 and using 1,2,3-triazole in the last step; mp=162-163° C.; ¹H NMR (300 MHz, CDCl₃) δ 9.05 (1H, s), 9.04 (1H, s), 8.42 (1H, s), 7.47 (2H, s), 5.83 (1H, m), 5.58 (1H, m), 5.3 (1H, dd, J=8.8 and 13.9 Hz), 5.15 (1H, dd, J=4.4 and 13.9 Hz), 5.11 (1H, dd, J=9.3 and 13.9 Hz), 4.95 (1H, dd, J=4.2 and 13.9 Hz), 2.29 (1H, m), 2.15 (1H, m), 1.81 (3H, d, J=7.2 Hz), and 1.04 (3H, t, J=7.2 Hz).

### Examples 87 and 88

### 3-[(2R)-1-(1H-Tetrazol-1-yl)propan-2-yl]-8-[(2R)-1-(2H-tetrazol-2-yl)propan-2-yl]-3,8-dihydrobenzo[1,2-d:4,5-d′]bis[1,2,3]triazine-4,9-dione and 3,8-Bis[(2R)-1-(1H-tetrazol-1-yl)propan-2-yl]-3,8-dihydrobenzo[1,2-d:4,5-d′]bis[1,2,3]triazine-4,9-dione

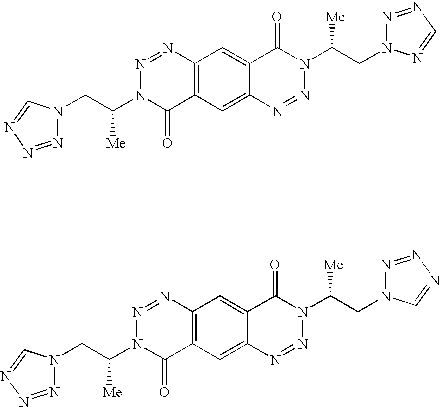

Intermediate 39 (5.00 g, 19.8 mmol) was added to a concentrated methanolic solution of HCl (200 ml), and the obtained suspension was stirred at rt. for 4 days. The reaction mixture was poured onto crushed ice (400 g), treated with solid sodium bicarbonate (→pH 8) and extracted with chloroform (2×400 ml). The extract was dried over sodium sulfate, and the solvent was removed under reduced pressure to give the desired diamine. A mixture of the diamine (4.26 g, 19 mmol), (R)-2-aminopropanol (5.00 g, 66 mmol), NaCN (0.49 g, 10 mmol) and methanol (5 ml) was stirred and heated in a stream of argon with distillation of the volatiles. After the distillation ceased, the mixture was heated at 140-150° C. for an additional 30 min. After cooling, the reaction mixture was dissolved in water (200 ml), saturated with NaCl and treated with 6N HCl to pH 5. After stirring for 30 min, the precipitate was filtered off, washed with water (20 ml) to give diamide. A solution of the preceding diamide (5.00 g, 14.5 mmol) in DMF (70 ml) was treated with isopentyl nitrite (8.0 ml, 60 mmol) and acetic acid (2 ml). The reaction mixture was stirred at room temperature for 16 h, and the volatiles were removed under reduced pressure. The product was purified by column chromatography (silica gel, chloroform/isopropanol, 4:1) and a solution of the product (1.20 g, 3.61 mmol) and pyridine (4.0 ml, 50 mmol) in dry chloroform (50 ml) was treated with methanesulfonic anhydride (2.61 g, 15 mmol). The mixture was stirred at rt. for 4 h, then poured onto crushed ice (100 g), acidified with 6N HCl to pH 1, and extracted with chloroform (3×100 ml). The extract was dried over magnesium sulfate, and the solvent was removed under reduced pressure to give a disulfonate. A solution of the disulfonate (1.76 g, 3.24 mmol) and tetrazole (1.50 g, 21.4 mmol) in DMF (20 ml) was stirred under argon and treated with 60% NaH (0.82 g, 20.5 mmol). The mixture was stirred and heated at 60° C. for 16 h and then it was poured into ice/water (100 ml), acidified to pH 3 with 1N HCl and extracted with chloroform (3×100 ml). The extract was dried over magnesium sulfate, concentrated and chromatographed (silica gel, ethyl acetate/ethanol/triethylamine, (95:3:2). The product was recrystallized from ethanol to give pure 3-[(2R)-1-(1H-tetrazol-1-yl)propan-2-yl]-8-[(2R)-1-(2H-tetrazol-2-yl)propan-2-yl]-3,8-dihydrobenzo[1,2-d:4,5-d′]bis[1,2,3]triazine-4,9-dione;

mp=160-161° C.; ¹H NMR (300 MHz, CDCl₃) δ 9.06 (2H, s), 8.61 (1H, s), 8.40 (1H, s), 5.82 (2H, m), 5.35 (1H, dd, J=8.8 and 13.9 Hz), 5.22 (1H, dd, J=9.5 and 14.3 Hz), 5.17 (1H, dd, J=4.4 and 13.9 Hz), 4.92 (1H, dd, J=5.1 and 14.3 Hz), 1.81 (3H, d, J=6.9 Hz), and 1.77 (3H, d, J=6.9 Hz).

The more polar product was identified as 3,8-bis[(2R)-1-(1H-tetrazol-1-yl)propan-2-yl]-3,8-dihydrobenzo[1,2-d:4,5-d′]bis[1,2,3]triazine-4,9-dione and was recrystallized from ethanol;

mp=220-222° C.; ¹H NMR (300 MHz, CDCl₃) δ 9.05 (2H, s), 8.62 (2H, s), 5.80 (2H, m), 5.21 (2H, dd, J=9.2 and 14.3 Hz), 4.92 (2H, dd, J=4.8 and 14.2 Hz), and 1.77 (6H, d, J=6.6 Hz).

### Example 89

### 3-(2-Hydroxy-2-methylpropyl)-8-[(2R)-1-(2H-tetrazol-2-yl)propan-2-yl]-3,8-dihydrobenzo[1,2-d:4,5-d′]bis[1,2,3]triazine-4,9-dione

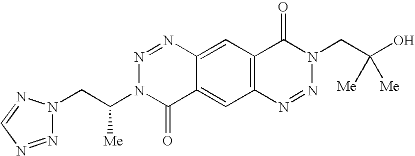

The title compound was prepared from intermediate 40 and 1,1-dimethylaminopropanol using the procedure for example 1; mp=128-130° C.; ¹H NMR (300 MHz, CDCl₃) δ 9.15 (1H, s), 9.07 (1H, s), 8.40 (1H, s), 5.84 (1H, m), 5.36 (1H, dd, J=8.8 and 13.9 Hz), 5.16 (1H, dd, J=4.4 and 13.9 Hz), 4.62 (2H, s), 2.68 (1H, s), 1.81 (3H, d, J=6.9 Hz), and 1.38 (6H, s).

### Biological Procedures

### Example 1

### In Vitro Physiological Testing

The physiological effects of invention compounds were tested in vitro on primary cultures of rat cortical or hippocampal neurons or on slices of rat hippocampus according to the following procedures.

Cortical cells were prepared from day 18-19 embryonic Sprague-Dawley rats and recorded after 3 days in culture. The extracellular solution (ECS) contained (in mM): NaCl (145), KCl (5.4), HEPES (10), MgCl2 (0.8), CaCl2 (1.8), glucose (10), sucrose (30); pH. 7.4. In order to block the voltage-gated sodium currents, 40 nM TTX was added to the recording solution. The intracellular solution contained (in mM): K-gluconate (140), HEPES (20), EGTA (1.1), phosphocreatine (5), MgATP (3), GTP (0.3), MgCl2 (5), and CaCl2 (0.1); pH: 7.2. All test compound and glutamate solutions were made-up in the extracellular solution.

The whole-cell current was measured with patch-clamp amplifier (Axopatch 200B), filtered at 2 kHz, digitized at 5 kHz and recorded on a PC with pClamp 8. The cells were voltage-clamped at −80 mV. Solutions were applied by DAD-12 system. A baseline response for each cell was recorded using a 1 s pulse of 500 μM glutamate dissolved in ECS. Responses to test compound were then determined by application of a 10 s pulse of test compound followed by a 1 s pulse of the same concentration of test compound plus 500 μM glutamate and then 10 s of saline. This pulse sequence was repeated until a stable reading was obtained, or until sufficient data points were measured to allow extrapolation to a calculated maximum change.

The mean value of plateau current between 600 ms to 900 ms after application of glutamate or test compound plus glutamate was calculated and used as the parameter to measure the drug effect. The plateau responses in the presence of varying concentrations of test compound were divided by the baseline response in order to calculate the percentage increase. Compounds are deemed active in this test if, at a test concentration of 3 μM or less, they produce a greater than 100% increase in the value of the steady-state current measured due to application of glutamate alone. The concentration at which the glutamate induced current is increased by 100% is commonly referred to as the EC2x value. Compounds of the examples disclosed above displayed EC2x values in the range of 0.05 to 10 μM.

In another test, excitatory responses (field EPSPs) were measured in hippocampal slices, which were maintained in a recording chamber continuously perfused with artificial cerebrospinal fluid (ACSF). During a 15-30 minute interval, the perfusion medium was switched to one containing various concentrations of the test compounds. Responses collected immediately before and at the end of drug perfusion were superimposed in order to calculate the percent increase in EPSP amplitude.

To conduct these tests, the hippocampus was removed from anesthetized, 2 month old Sprague-Dawley rats and in vitro slices (400 μm thick) were prepared and maintained in an interface chamber at 35° C. using conventional techniques [see, for example, Dunwiddie and Lynch, J. Physiol. 276: 353-367 (1978)]. The chamber was constantly perfused at 0.5 mL/min with ACSF containing (in mM): NaCl 124, KCl 3, KH₂PO₄ 1.25, MgSO₄ 2.5, CaCl₂ 3.4, NaHCO3 26, glucose 10 and L-ascorbate 2. A bipolar nichrome stimulating electrode was positioned in the dendritic layer (stratum radiatum) of the hippocampal subfield CA1 close to the border of subfield CA3.

Current pulses (0.1 msec) through the stimulating electrode activate a population of the Schaffer-commissural (SC) fibers, which arise from neurons in the subdivision CA3 and terminate in synapses on the dendrites of CA1 neurons. Activation of these synapses causes them to release the transmitter glutamate. Glutamate binds to the post-synaptic AMPA receptors, which then transiently open an associated ion channel and permit a sodium current to enter the postsynaptic cell. This current results in a voltage in the extracellular space (the field EPSP) which is recorded by a high impedance recording electrode positioned in the middle of the stratum radiatum of CA1. The intensity of the stimulation current was adjusted to produce half-maximal EPSPs (typically about 1.5-2.0 mV). Paired stimulation pulses were given every 40 s with an interpulse interval of 200 msec (see below). The field EPSPs of the second response were digitized and analyzed to determine amplitude. If the responses were stable for 15-30 min (baseline), test compounds were added to the perfusion lines for a period of about 15 min. The perfusion was then changed back to regular ACSF.

Paired-pulse stimulation was used since stimulation of the SC fibers, in part, activates interneurons which generate an inhibitory postsynaptic potential (IPSP) in the pyramidal cells of CAl. This feed forward IPSP typically sets in after the EPSP reaches its peak. It accelerates the repolarization and shortens the decay phase of the EPSP, and thus could partially mask the effects of the test compounds. One of the relevant features of the feed-forward IPSP is that it can not be reactivated for several hundred milliseconds following a stimulation pulse. This phenomenon can be employed to advantage to eliminate IPSP by delivering paired pulses separated by 200 ms and using the second (“primed”) response for data analysis. Compounds are deemed active in this test if, at a test concentration of 10 μM or less, they produce a greater than 10% increase in the value of the evoked current measured for the baseline period (herein referred to as EC_{15%}). Compounds of the examples disclosed above displayed values for EC_{15%} in the range of 0.1 to 20 μM.

### Example 2

### In Vivo Physiological Testing

The physiological effects of invention compounds were tested in vivo in anesthetized animals according to the following procedures.

Animals are maintained under anesthesia by phenobarbital administered using a Hamilton syringe pump. Stimulating and recording electrodes are inserted into the perforant path and dentate gyrus of the hippocampus, respectively. Once electrodes are implanted, a stable baseline of evoked responses are elicited using single monophasic pulses (100 μs pulse duration) delivered at 3/min to the stimulating electrode. Field EPSPs are monitored until a stable baseline is achieved (about 20-30 min), after which a solution of test compound in HPCD is injected intraperitoneally and evoked field potentials are recorded. Evoked potentials are recorded for approximately 2 h following drug administration or until the amplitude of the field EPSP returns to baseline. In the latter instance, it is common that an iv administration is also carried out with an appropriate dose of the same test compound.

While the invention has been described with reference to specific methods and embodiments, it will be appreciated that various modifications may be made without departing from the invention.

1. A compound according to the formula:
wherein: 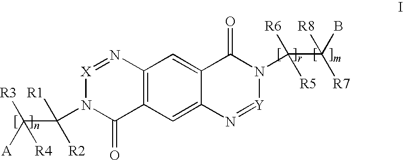
X═C—H or N,
Y═C—H or N, with the proviso that Y cannot be carbon when X═N and the group 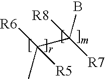
represents H, alkyl or cycloalkyl,
R¹ and R² are independently hydrogen, alkyl, substituted alkyl, cycloalkyl, substituted cycloalkyl, alkynyl, substituted alkynyl, alkenyl, substituted alkenyl, cyano, alkoxy, and if R¹ and R² are alkyl, R¹ and R² may be joined with a bond or —(CH₂)ₚ— to produce a cycloalkyl,
R³ and R⁴ are independently hydrogen, alkyl, substituted alkyl, cycloalkyl, substituted cycloalkyl, alkynyl, substituted alkynyl, alkenyl, substituted alkenyl, hydroxyl, alkoxy, cyano, fluoro,
A may be absent, hydrogen, alkyl, substituted alkyl, cycloalkyl, substituted cycloalkyl, alkynyl, substituted alkynyl, alkenyl, substituted alkenyl, aromatic, substituted aromatic, heteroaromatic, substituted heteroaromatic, bicycloheteroaromatic, heterocycle, substituted heterocycle, hydroxyl, alkoxy, cyano, fluoro, SCN, SO₂NR⁹R¹⁰, CONR⁹R¹⁰, NR¹¹SO₂R¹², NR¹¹COR¹², OR⁹, NR⁹R¹⁰,
R⁵ and R⁶ are independently hydrogen, alkyl, substituted alkyl, cycloalkyl, substituted cycloalkyl, alkynyl, substituted alkynyl, alkenyl, substituted alkenyl, cyano, alkoxy, and if R⁵ and R⁶ are alkyl, R⁵ and R⁶ may be joined with a bond or —(CH₂)ₚ— to produce a cycloalkyl,
R⁷ and R⁸ are independently hydrogen, alkyl, substituted alkyl, cycloalkyl, substituted cycloalkyl, alkynyl, substituted alkynyl, alkenyl, substituted alkenyl, hydroxyl, alkoxy, cyano, fluoro,
R⁹, R¹⁰, R¹¹ and R¹², are independently hydrogen, alkyl, substituted alkyl, cycloalkyl, substituted cycloalkyl, alkynyl, substituted alkynyl, alkenyl, substituted alkenyl, or
R⁹ and R¹⁰, and R¹¹ and R¹² may be joined with a bond or —(CH₂)_{q}— to produce a cycloalkyl,
B may be absent, hydrogen, alkyl, substituted alkyl, cycloalkyl, substituted cycloalkyl, alkynyl, substituted alkynyl, alkenyl, substituted alkenyl, aromatic, substituted aromatic, heteroaromatic, substituted heteroaromatic, bicycloheteroaromatic, heterocycle, substituted heterocycle, hydroxyl, alkoxy, cyano, fluoro, SCN, SO₂NR⁹R¹⁰, CONR⁹R¹⁰, NR¹¹SO₂R¹², NR¹¹COR¹², OR⁹, NR⁹R¹⁰,
n=0, 1 or 2,
m=0, 1 or 2,
p=1, 2, or 3,
q=2, 3 or 4,
r=0 or 1 or a pharmaceutically acceptable salt, solvate or polymorph thereof.

2. A compound of claim 1 according to the formula: 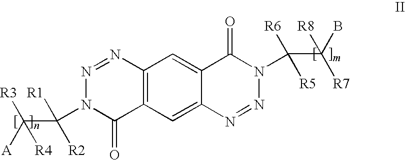
wherein:
R¹, R², R³, R⁴, R⁵, R⁶, A, B, n and m are as defined for formula I, or a pharmaceutically acceptable salt, solvate or polymorph thereof.

3. A compound of claim 1 according to the formula: 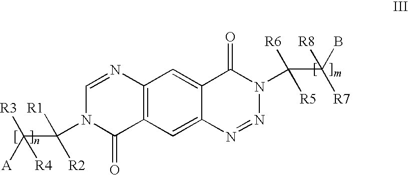
wherein:
R¹, R², R³, R⁴, R⁵, R⁶, A, B, n and m are as defined for formula I, or a pharmaceutically acceptable salt, solvate or polymorph thereof.

4. A compound of claim 1 according to the formula: 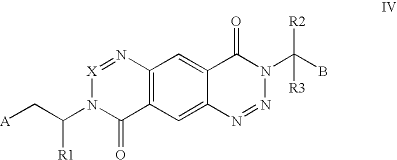
wherein:
X═C—H or N,
R¹ is hydrogen, alkyl, substituted alkyl, cycloalkyl, substituted cycloalkyl, alkenyl, substituted alkenyl, alkynyl, substituted alkynyl,
R² and R³ are independently hydrogen, alkyl, substituted alkyl, cycloalkyl, substituted cycloalkyl, alkenyl, substituted alkenyl, alkynyl, substituted alkynyl, and R² and R³ may be joined with a bond or —(CH₂)ₚ— to produce a cycloalkyl,
Particular preferred groups for A include but are not limited to aromatic, substituted aromatic, 5-ring heteroaromatics, substituted 5-ring heteroaromatics, 6-ring heteroaromatics, substituted 6-ring heteroaromatics,
p=1, 2 or 3,
B may be absent, hydrogen, alkyl, substituted alkyl, cycloalkyl, substituted cycloalkyl, alkynyl, substituted alkynyl, alkenyl, substituted alkenyl, aromatic, substituted aromatic, 5-ring heteroaromatics, substituted 5-ring heteroaromatics, 6-ring heteroaromatics, substituted 6-ring heteroaromatics, or a pharmaceutically acceptable salt, solvate or polymorph thereof.

5. A compound of claim 1 according to the formula: 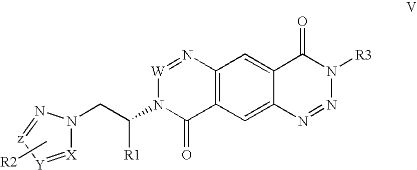
wherein:
W═C—H or N,
X, Y and Z are independently C—H or N to a maximum of 4 N's in the ring,
R¹ is hydrogen, methyl, ethyl, acetylene, cyclopropyl, fluoro,
R² is hydrogen, methyl, ethyl, CF₃,
R³ is methyl, ethyl, cyclopropyl, isopropyl, —(CH₂)ₚCCH, —(CH₂)ₚOR⁴, —(CH₂)ₚCN,
R⁴ is H, alkyl, substituted alkyl, cycloalkyl, substituted cycloalkyl, alkynyl, substituted alkynyl, alkenyl, substituted alkenyl, aromatic, substituted aromatic,
p=0, 1, 2 or 3, or a pharmaceutically acceptable salt, solvate or polymorph thereof.

6. A compound of claim 1 according to the formula: 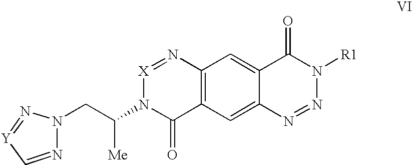
wherein:
X═C—H or N,
Y═C—H or N,
R¹ is methyl, ethyl, cyclopropyl, isopropyl, —(CH₂)ₚCCH, —(CH₂)ₚOR², —(CH₂)ₚCN,
R² is H, alkyl, substituted alkyl, cycloalkyl, substituted cycloalkyl, alkynyl, substituted alkynyl, alkenyl, substituted alkenyl, aromatic, substituted aromatic,
p=0, 1, 2 or 3, or a pharmaceutically acceptable salt, solvate or polymorph thereof.

7. A compound of claim 1 according to the formula: 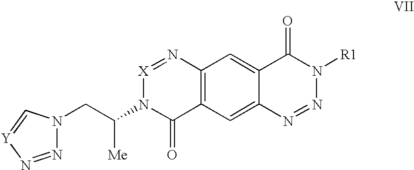
wherein:
X═C—H or N,
Y═C—H or N,
R¹ is methyl, ethyl, cyclopropyl, isopropyl, —(CH₂)ₚCCH, —(CH₂)ₚOR², —(CH₂)ₚCN,
R² is H, alkyl, substituted alkyl, cycloalkyl, substituted cycloalkyl, alkynyl, substituted alkynyl, alkenyl, substituted alkenyl, aromatic, substituted aromatic,
p=0, 1, 2 or 3, or a pharmaceutically acceptable salt, solvate or polymorph thereof.

8. A compound of claim 1 according to the formula: 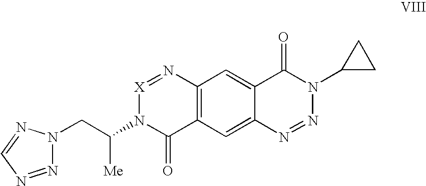
wherein:
X═C—H or N, or a pharmaceutically acceptable salt, solvate or polymorph thereof.

9. A compound of claim 1 according to the formula: 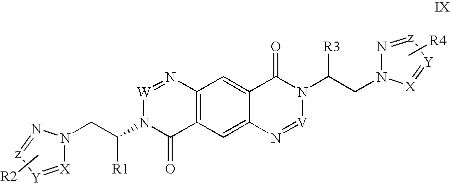
wherein:
V and W are independently C—H or N,
X, Y and Z are independently C—H or N to a maximum of 4 N's in the ring,
R¹ and R3 are independently hydrogen, methyl, ethyl, acetylene, cyclopropyl, fluoro,
R² and R4 are independently hydrogen, methyl, ethyl, CF₃, or a pharmaceutically acceptable salt, solvate or polymorph thereof.

10. A compound of claim 1 according to the formula: 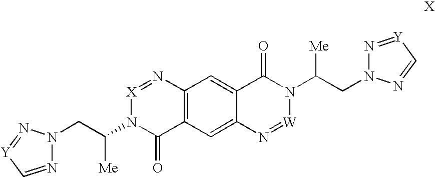
wherein:
X and W are independently C—H or N,
Y═C—H or N, or a pharmaceutically acceptable salt, solvate or polymorph thereof.

11. A compound of claim 1 according to the formula: 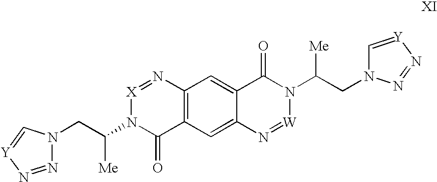
wherein:
X and W are independently C—H or N,
Y═C—H or N, or a pharmaceutically acceptable salt, solvate or polymorph thereof.

12. A compound of claim 1 according to Formulas I-XI which is:
3-Cyclopropyl-8-[(1R)-1-methyl-2-(2H-tetrazol-2-yl)ethyl]-3,8-dihydro[1,2,3]triazino[4,5-g][1,2,3]benzotriazine-4,9-dione
3-Cyclopropyl-8-[(2R)-1-(5-methyl-2H-tetrazol-2-yl)propan-2-yl]-3,8-dihydrobenzo[1,2-d:4,5-d′]bis[1,2,3]triazine-4,9-dione
3-Cyclopropyl-8-[(2R)-1-(5-methyl-1H-tetrazol-1-yl)propan-2-yl]-3,8-dihydrobenzo[1,2-d:4,5-d′]bis[1,2,3]triazine-4,9-dione
3-Methyl-8-[(2R)-1-(2H-tetrazol-2-yl)propan-2-yl]-3,8-dihydrobenzo[1,2-d:4,5-d′]bis[1,2,3]triazine-4,9-dione
3-Methyl-8-[(2R)-1-(1H-tetrazol-1-yl)propan-2-yl]-3,8-dihydrobenzo[1,2-d:4,5-d′]bis[1,2,3]triazine-4,9-dione
3-Ethyl-8-[(2R)-1-(2H-tetrazol-2-yl)propan-2-yl]-3,8-dihydrobenzo[1,2-d:4,5-d′]bis[1,2,3]triazine-4,9-dione
3-(2-Fluoroethyl)-8-[(2R)-1-(2H-tetrazol-2-yl)propan-2-yl]-3,8-dihydrobenzo[1,2-d:4,5-d′]bis[1,2,3]triazine-4,9-dione
3-Propan-2-yl-8-[(2R)-1-(2H-tetrazol-2-yl)propan-2-yl]-3,8-dihydrobenzo[1,2-d:4,5-d′]bis[1,2,3]triazine-4,9-dione
3-Cyclobutyl-8-[(2R)-1-(2H-tetrazol-2-yl)propan-2-yl]-3,8-dihydrobenzo[1,2-d:4,5-d′]bis[1,2,3]triazine-4,9-dione
3-(Cyclopropylmethyl)-8-[(2R)-1-(2H-tetrazol-2-yl)propan-2-yl]-3,8-dihydrobenzo[1,2-d:4,5-d′]bis[1,2,3]triazine-4,9-dione
3-(2-Methylpropyl)-8-[(2R)-1-(2H-tetrazol-2-yl)propan-2-yl]-3,8-dihydrobenzo[1,2-d:4,5-d′]bis[1,2,3]triazine-4,9-dione
3-But-3-yn-1-yl-8-[(2R)-1-(2H-tetrazol-2-yl)propan-2-yl]-3,8-dihydrobenzo[1,2-d:4,5-d′]bis[1,2,3]triazine-4,9-dione
3-But-3-yn-1-yl-8-[(2R)-1-(1H-tetrazol-1-yl)propan-2-yl]-3,8-dihydrobenzo[1,2-d:4,5-d′]bis[1,2,3]triazine-4,9-dione
3-Cyclopropyl-8-(1-pyridin-3-ylpropan-2-yl)-3,8-dihydrobenzo[1,2-d:4,5-d′]bis[1,2,3]triazine-4,9-dione
3-Cyclopropyl-8-[2-(2H-tetrazol-2-yl)ethyl]-3,8-dihydrobenzo[1,2-d:4,5-d′]bis[1,2,3]triazine-4,9-dione
3-Cyclopropyl-8-[(2R)-1-(1H-tetrazol-1-yl)propan-2-yl]-3,8-dihydrobenzo[1,2-d:4,5-d′]bis[1,2,3]triazine-4,9-dione
3-Cyclopropyl-8-[(2R)-1-(2H-tetrazol-2-yl)propan-2-yl]-3,8-dihydropyrimido[4,5-g]quinazoline-4,9-dione
3-(2-Methoxyethyl)-8-[(2R)-1-(2H-tetrazol-2-yl)propan-2-yl]-3,8-dihydrobenzo[1,2-d:4,5-d′]bis[1,2,3]triazine-4,9-dione
3-(2-Methoxyethyl)-8-[(2R)-1-(2H-tetrazol-2-yl)propan-2-yl]-3,8-dihydro[1,2,3]triazino[4,5-g]quinazoline-4,9-dione
3-(Pyridin-3-ylmethyl)-8-[(2R)-1-(2H-tetrazol-2-yl)propan-2-yl]-3,8-dihydrobenzo[1,2-d:4,5-d′]bis[1,2,3]triazine-4,9-dione
3-Prop-2-yn-1-yl-8-[(2R)-1-(2H-tetrazol-2-yl)propan-2-yl]-3,8-dihydrobenzo[1,2-d:4,5-d′]bis[1,2,3]triazine-4,9-dione
{4,9-Dioxo-8-[(2R)-1-(2H-tetrazol-2-yl)propan-2-yl]-8,9-dihydrobenzo[1,2-d:4,5-d′]bis[1,2,3]triazin-3(4H)-yl}acetonitrile
3-Cyclopropyl-8-[(2R)-1-(2H-tetrazol-2-yl)but-3-yn-2-yl]-3,8-dihydrobenzo[1,2-d:4,5-d′]bis[1,2,3]triazine-4,9-dione
3-Cyclopropyl-8-[(2R)-1-(1H-tetrazol-1-yl)but-3-yn-2-yl]-3,8-dihydrobenzo[1,2-d:4,5-d′]bis[1,2,3]triazine-4,9-dione
3-Cyclopropyl-8-[(2R)-1-(2H-1,2,3-triazol-2-yl)propan-2-yl]-3,8-dihydrobenzo[1,2-d:4,5-d′]bis[1,2,3]triazine-4,9-dione
3-Cyclopropyl-8-[(2R)-1-(1H-1,2,3-triazol-1-yl)propan-2-yl]-3,8-dihydrobenzo[1,2-d:4,5-d′]bis[1,2,3]triazine-4,9-dione
3-Methyl-8-[(2R)-1-(2H-1,2,3-triazol-2-yl)propan-2-yl]-3,8-dihydrobenzo[1,2-d:4,5-d′]bis[1,2,3]triazine-4,9-dione
3-Methyl-8-[(2R)-1-(1H-1,2,3-triazol-1-yl)propan-2-yl]-3,8-dihydrobenzo[1,2-d:4,5-d′]bis[1,2,3]triazine-4,9-dione
3-(2-Methoxyethyl)-8-[(2R)-1-(2H-1,2,3-triazol-2-yl)propan-2-yl]-3,8-dihydrobenzo[1,2-d:4,5-d′]bis[1,2,3]triazine-4,9-dione
3-Cyclopropyl-8-[(2R)-1-(1H-1,2,4-triazol-1-yl)propan-2-yl]-3,8-dihydrobenzo[1,2-d:4,5-d′]bis[1,2,3]triazine-4,9-dione
3-Cyclopropyl-8-[1-(1H-pyrazol-1-yl)propan-2-yl]-3,8-dihydrobenzo[1,2-d:4,5-d′]bis[1,2,3]triazine-4,9-dione
3-Methyl-8-[1-(1H-pyrazol-1-yl)propan-2-yl]-3,8-dihydrobenzo[1,2-d:4,5-d′]bis[1,2,3]triazine-4,9-dione
(2R)-2-(8-Cyclopropyl-4,9-dioxo-8,9-dihydrobenzo[1,2-d:4,5-d′]bis[1,2,3]triazin-3(4H)-yl)propyl thiocyanate
3-But-2-yn-1-yl-8-[(2R)-1-(2H-tetrazol-2-yl)propan-2-yl]-3,8-dihydrobenzo[1,2-d:4,5-d′]bis[1,2,3]triazine-4,9-dione
N-[(2R)-2-(8-Cyclopropyl-4,9-dioxo-8,9-dihydrobenzo[1,2-d:4,5-d′]bis[1,2,3]triazin-3(4H)-yl)propyl]methanesulfonamide
N-[(2R)-2-(8-Cyclopropyl-4,9-dioxo-8,9-dihydrobenzo[1,2-d:4,5-d′]bis[1,2,3]triazin-3(4H)-yl)propyl]-N-methylmethanesulfonamide
3-Cyclopropyl-8-[(2S)-1-(3-fluorophenyl)but-3-yn-2-yl]-3,8-dihydrobenzo[1,2-d:4,5-d′]bis[1,2,3]triazine-4,9-dione
3-[(2R)-1-(2H-Tetrazol-2-yl)propan-2-yl]-8-[(2R)-1-(2H-1,2,3-triazol-2-yl)propan-2-yl]-3,8-dihydrobenzo[1,2-d:4,5-d′]bis[1,2,3]triazine-4,9-dione
3-[(2R)-1-(2H-Tetrazol-2-yl)propan-2-yl]-8-[(2R)-1-(1H-1,2,3-triazol-1-yl)propan-2-yl]-3,8-dihydrobenzo[1,2-d:4,5-d′]bis[1,2,3]triazine-4,9-dione
3-[(2R)-1-(2H-Tetrazol-2-yl)propan-2-yl]-8-[(2S)-1-(2H-tetrazol-2-yl)propan-2-yl]-3,8-dihydrobenzo[1,2-d:4,5-d′]bis[1,2,3]triazine-4,9-dione
3-[(2R)-1-(2H-Tetrazol-2-yl)propan-2-yl]-8-[(2S)-1-(1H-tetrazol-1-yl)propan-2-yl]-3,8-dihydrobenzo[1,2-d:4,5-d′]bis[1,2,3]triazine-4,9-dione
3-Methoxy-8-[(2R)-1-(2H-tetrazol-2-yl)propan-2-yl]-3,8-dihydrobenzo[1,2-d:4,5-d′]bis[1,2,3]triazine-4,9-dione
3-(3-Methoxypropyl)-8-[(2R)-1-(2H-tetrazol-2-yl)propan-2-yl]-3,8-dihydrobenzo[1,2-d:4,5-d′]bis[1,2,3]triazine-4,9-dione
3-Prop-2-en-1-yl-8-[(2R)-1-(2H-tetrazol-2-yl)propan-2-yl]-3,8-dihydrobenzo[1,2-d:4,5-d′]bis[1,2,3]triazine-4,9-dione
(3R)-3-(8-Cyclopropyl-4,9-dioxo-8,9-dihydrobenzo[1,2-d:4,5-d′]bis[1,2,3]triazin-3(4H)-yl)butanenitrile
(3R)-3-{4,9-Dioxo-8-[(2R)-1-(2H-1,2,3-triazol-2-yl)propan-2-yl]-8,9-dihydrobenzo[1,2-d:4,5-d′]bis[1,2,3]triazin-3(4H)-yl}butanenitrile
(3R)-3-{4,9-Dioxo-8-[(2R)-1-(2H-tetrazol-2-yl)propan-2-yl]-8,9-dihydrobenzo[1,2-d:4,5-d′]bis[1,2,3]triazin-3 (4H)-yl}butanenitrile
3-But-2-yn-1-yl-8-[(2R)-1-(2H-tetrazol-2-yl)propan-2-yl]-3,8-dihydrobenzo[1,2-d:4,5-d′]bis[1,2,3]triazine-4,9-dione
3-But-2-yn-1-yl-8-[(2R)-1-(2H-1,2,3-triazol-2-yl)propan-2-yl]-3,8-dihydrobenzo[1,2-d:4,5-d′]bis[1,2,3]triazine-4,9-dione
3-Pent-3-yn-2-yl-8-[(2R)-1-(2H-tetrazol-2-yl)propan-2-yl]-3,8-dihydrobenzo[1,2-d:4,5-d′]bis[1,2,3]triazine-4,9-dione
3,8-Bis[(2R)-1-(2H-1,2,3-triazol-2-yl)propan-2-yl]-3,8-dihydrobenzo[1,2-d:4,5-d′]bis[1,2,3]triazine-4,9-dione
3,8-Bis[(2R)-1-(1H-1,2,3-triazol-1-yl)propan-2-yl]-3,8-dihydrobenzo[1,2-d:4,5-d′]bis[1,2,3]triazine-4,9-dione
3-Cyclopropyl-8-[(2R)-1-(2H-tetrazol-2-yl)propan-2-yl]-3,8-dihydro[1,2,3]triazino[4,5-g]quinazoline-4,9-dione
3-Cyclopropyl-8-[(2R)-1-(1H-tetrazol-1-yl)propan-2-yl]-3,8-dihydro[1,2,3]triazino[4,5-g]quinazoline-4,9-dione
8-[(2R)-1-(1H-Benzotriazol-1-yl)propan-2-yl]-3-cyclopropyl-3,8-dihydro[1,2,3]triazino[4,5-g]quinazoline-4,9-dione
3-Cyclopropyl-8-[(2R)-1-(2H-tetrazol-2-yl)butan-2-yl]-3,8-dihydro[1,2,3]triazino[4,5-g]quinazoline-4,9-dione
3-Cyclopropyl-8-[(2R)-1-(1H-tetrazol-1-yl)butan-2-yl]-3,8-dihydro[1,2,3]triazino[4,5-g]quinazoline-4,9-dione
3-Cyclopropyl-8-[(2R)-1-(2H-1,2,3-triazol-2-yl)butan-2-yl]-3,8-dihydro[1,2,3]triazino[4,5-g]quinazoline-4,9-dione
3-Cyclopropyl-8-[(2R)-1-(1H-1,2,3-triazol-1-yl)butan-2-yl]-3,8-dihydro[1,2,3]triazino[4,5-g]quinazoline-4,9-dione
3-Prop-2-yn-1-yl-8-[(2R)-1-(2H-tetrazol-2-yl)propan-2-yl]-3,8dihydro[1,2,3]triazino[4,5-g]quinazoline-4,9-dione
3-Prop-2-yn-1-yl-8-[(2R)-1-(1H-tetrazol-1-yl)propan-2-yl]-3,8-dihydro[1,2,3]triazino[4,5-g]quinazoline-4,9-dione
8-[(2R)-1-(2H-Tetrazol-2-yl)propan-2-yl]-3-(1H-1,2,3-triazol-4-ylmethyl)-3,8-dihydro[1,2,3]triazino[4,5-g]quinazoline-4,9-dione
3-Cyclopropyl-8-[2-(2H-tetrazol-2-yl)ethyl]-3,8-dihydro[1,2,3]triazino[4,5-g]quinazoline-4,9-dione
3,8-Bis[(2R)-1-(2H-tetrazol-2-yl)propan-2-yl]-3,8-dihydro[1,2,3]triazino[4,5-g]quinazoline-4,9-dione
3-[(2R)-1-(1H-Tetrazol-1-yl)propan-2-yl]-8-[(2R)-1-(2H-tetrazol-2-yl)propan-2-yl]-3,8-dihydro[1,2,3]triazino[4,5-g]quinazoline-4,9-dione
8-[(2R)-1-(2H-Tetrazol-2-yl)butan-2-yl]-3-[(2R)-1-(2H-tetrazol-2-yl)propan-2-yl]-3,8-dihydro[1,2,3]triazino[4,5-g]quinazoline-4,9-dione
8-[(2R)-1-(1H-Tetrazol-1-yl)butan-2-yl]-3-[(2R)-1-(2H-tetrazol-2-yl)propan-2-yl]-3,8-dihydro[1,2,3]triazino[4,5-g]quinazoline-4,9-dione
8-[(2R)-1-(5-Methyl-2H-tetrazol-2-yl)butan-2-yl]-3-[(2R)-1-(2H-tetrazol-2-yl)propan-2-yl]-3,8-dihydro[1,2,3]triazino[4,5-g]quinazoline-4,9-dione
8-[(2R)-1-(5-Methyl-1H-tetrazol-1-yl)butan-2-yl]-3-[(2R)-1-(2H-tetrazol-2-yl)propan-2-yl]-3,8dihydro[1,2,3]triazino[4,5-g]quinazoline-4,9-dione
3,8-Bis[(2R)-1-(2H-tetrazol-2-yl)butan-2-yl]-3,8-dihydro[1,2,3]triazino[4,5-g]quinazoline-4,9-dione
3,8-Bis[(2R)-1-(1H-tetrazol-1-yl)butan-2-yl]-3,8dihydro[1,2,3]triazino[4,5-g]quinazoline-4,9-dione
3,8-Bis[(2R)-1-(2H-tetrazol-2-yl)butan-2-yl]-3,8-dihydropyrimido[4,5-g]quinazoline-4,9-dione
3-[(2R)-1-Hydroxybutan-2-yl]-8-[(2R)-1-(2H-tetrazol-2-yl)butan-2-yl]-3,8-dihydropyrimido[4,5-g]quinazoline-4,9-dione
3-[(2R)-1-(2H-Tetrazol-2-yl)butan-2-yl]-8-[(2R)-1-(4H-1,2,4-triazol-4-yl)butan-2-yl]-3,8-dihydropyrimido[4,5-g]quinazoline-4,9-dione
3,8-Bis[(2R)-1-(2H-tetrazol-2-yl)propan-2-yl]-3,8-dihydropyrimido[4,5-g]quinazoline-4,9-dione
3,8-Bis[(2R)-1-(1H-pyrazol-1-yl)propan-2-yl]-3,8-dihydropyrimido[4,5-g]quinazoline-4,9-dione
3-[(2R)-1-(4-Chloro-1H-pyrazol-1-yl)propan-2-yl]-8-[(2R)-1-(1H-pyrazol-1-yl)propan-2-yl]-3,8-dihydropyrimido[4,5-g]quinazoline-4,9-dione
3,8-Bis[2-(3-fluorophenyl)ethyl]-3,8-dihydropyrimido[4,5-g]quinazoline-4,9-dione
3-[(2R)-1-Hydroxypropan-2-yl]-8-[(2R)-1-(2H-tetrazol-2-yl)propan-2-yl]-3,8-dihydrobenzo[1,2-d:4,5-d′]bis[1,2,3]triazine-4,9-dione
3,8-Bis[(2R)-1-(2H-tetrazol-2-yl)propan-2-yl]-3,8-dihydrobenzo[1,2-d:4,5-d′]bis[1,2,3]triazine-4,9-dione
3-[(2R)-1-(5-Methyl-2H-tetrazol-2-yl)propan-2-yl]-8-[(2R)-1-(2H-tetrazol-2-yl)propan-2-yl]-3,8-dihydrobenzo[1,2-d:4,5-d′]bis[1,2,3]triazine-4,9-dione
3-[(2R)-1-(5-Methyl-1H-tetrazol-1-yl)propan-2-yl]-8-[(2R)-1-(2H-tetrazol-2-yl)propan-2-yl]-3,8-dihydrobenzo[1,2-d:4,5-d′]bis[1,2,3]triazine-4,9-dione
3-[(2R)-1-(2H-Tetrazol-2-yl)propan-2-yl]-8-{(2R)-1-[3-(trifluoromethyl)-1H-pyrazol-1-yl]propan-2-yl}-3,8-dihydrobenzo[1,2-d:4,5-d′]bis[1,2,3]triazine-4,9-dione
3-[(2R)-1-(2H-Tetrazol-2-yl)butan-2-yl]-8-[(2R)-1-(2H-tetrazol-2-yl)propan-2-yl]-3,8-dihydrobenzo[1,2-d:4,5-d′]bis[1,2,3]triazine-4,9-dione
3-[(2R)-1-(1H-Tetrazol-1-yl)butan-2-yl]-8-[(2R)-1-(2H-tetrazol-2-yl)propan-2-yl]-3,8-dihydrobenzo[1,2-d:4,5-d′]bis[1,2,3]triazine-4,9-dione
3-[(2R)-1-(2H-Tetrazol-2-yl)propan-2-yl]-8-[(2R)-1-(2H-1,2,3-triazol-2-yl)butan-2-yl]-3,8-dihydrobenzo[1,2-d:4,5-d′]bis[1,2,3]triazine-4,9-dione
3-[(2R)-1-(1H-Tetrazol-1-yl)propan-2-yl]-8-[(2R)-1-(2H-tetrazol-2-yl)propan-2-yl]-3,8-dihydrobenzo[1,2-d:4,5-d′]bis[1,2,3]triazine-4,9-dione
3,8-Bis[(2R)-1-(1H-tetrazol-1-yl)propan-2-yl]-3,8-dihydrobenzo[1,2-d:4,5-d′]bis[1,2,3]triazine-4,9-dione
3-(2-Hydroxy-2-methylpropyl)-8-[(2R)-1-(2H-tetrazol-2-yl)propan-2-yl]-3,8-dihydrobenzo[1,2-d:4,5-d′]bis[1,2,3]triazine-4,9-dione

13. A pharmaceutical composition comprising an effective amount of a compound according to claim 1 in combination with a pharmaceutically acceptable carrier, additive or excipient.

14. The composition according to claim 13 wherein said compound comprises about 0.5% to about 75% by weight of said composition and said carrier, additive or excipient comprises about 25% to about 95.5% of said composition.

15. A method for the treatment of a mammalian subject, wherein the subject suffers from a hypoglutamatergic condition or a deficiency in the number or strength of excitatory synapses or in the number of AMPA receptors, such that memory or other cognitive functions are impaired, said method comprising administering to said subject, in a pharmaceutically acceptable carrier, an effective amount of a compound according to claim 1.

16. A method for the treatment of a mammal wherein the subject suffers from a hypoglutamatergic condition or deficiencies in the number or strength of excitatory synapses or in the number of AMPA receptors such that a cortical/striatal imbalance occurs leading to schizophrenia or schizophreniform behavior, said method comprising administering to said subject, in a pharmaceutically acceptable carrier, an effective amount of a compound according to claim 1.

17. The method according to claim 15 wherein said condition is schizophrenia

18. The method according to claim 15 wherein said condition is Parkinson's disease.

19. A method of treating ADHD in a patient in need thereof, said method comprising administering to said patient an effective amount of a compound according to claim 1.

20. A method of treating Rett Syndrome in a patient in need thereof, said method comprising administering to said patient an effective amount of a compound according to claim 1.

21. A method of treating Fragile-X Syndrome in a patient in need thereof, said method comprising administering to said patient an effective amount of a compound according to claim 1.

22. A method of treating respiratory depression in a patient in need thereof, said method comprising administering to said patient an effective amount of a compound according to claim 1.

23. A method of treating respiratory depression in a patient in need thereof, said method comprising administering to said patient an effective amount of a compound according to claim 1 in combination with an opiate or opioid analgesic.

24. A method of treating respiratory depression in a patient in need thereof, said method comprising administering to said patient an effective amount of a compound according to claim 1 in combination with an anesthetic agent such as propofol or barbiturates.

25. A method of treating breathing related sleep disorders or sleep apnea in a patient in need thereof, said method comprising administering to said patient an effective amount of a compound according to claim 1.

26. A method of treating Alzheimer's disease in a patient in need thereof, said method comprising administering to said patient an effective amount of a compound according to claim 1.

27. A method of treating Alzheimer's disease in a patient in need thereof, said method comprising administering to said patient an effective amount of a compound according to claim 1 in combination with acetylcholinesterase inhibitors.

28. A method of treating Depression in a patient in need thereof, said method comprising administering to said patient an effective amount of a compound according to claim 1.

29. A method of treating Huntington's Disease in a patient in need thereof, said method comprising administering to said patient an effective amount of a compound according to claim 1.

30. A method of treating recovery from Stroke in a patient in need thereof, said method comprising administering to said patient an effective amount of a compound according to claim 1.

31. A method of treating recovery from Traumatic Brain Injury in a patient in need thereof, said method comprising administering to said patient an effective amount of a compound according to claim 1.

32-48. (canceled)
